(19) United States
(12) Patent Application Publication
(10) Pub. No.: US 2005/0112100 A1
(43) Pub. Date: May 26, 2005

(54) Sp1 and Sp3 targeted cancer therapies and therapeutics

(75) Inventors: J. Justin McCormack, Morrice, MI (US); Zhenjun Lou, Okemos, MI (US); Hongyan Liang, San Marcos, CA (US); Veronica M. Maher, E. Lansing, MI (US)

Correspondence Address: Ann-Louise Kerner, Ph.D;Wilmer Cutler Pickering Hale and Dorr LLP, 60 State Street, Boston, MA, 02109, US

(73) Assignee: Michigan State University, East Lansing, MI (US)

(21) Appl. No.: 10/888,875

(22) Filed: Jul. 9, 2004

Related U.S. Application Data
(60) Provisional application No. 60/486,500, filed on Jul. 10, 2003.

(51) Int. Cl. A61K 48/00

(52) U.S. Cl. 424/93.2; 514/44

(57) ABSTRACT

The invention provides methods and compositions for treating a cancerous condition, using one or more inhibitory agents that target the transcription factors Sp1 or Sp3. Compositions provided include Sp1 and Sp3—targeted antisense oligonucleotides and ribozymes and recombinant viral vectors for delivery of these ribozymes to a tumor, as well as Sp1/Sp3 specific promoters for optimized negative-feedback expression of the Sp1/Sp3 ribozymes. The invention further provides a system for analyzing the effectiveness of a cancer therapy treatment on a cancer by using an animal model in which the cancerous cells and recombinant cancer therapeutics are separately marked with different fluorescent markers that allow for the detection of effectively treated cancer cells and tissue.

### RELATED APPLICATIONS

This applications claims priority to U.S. provisional application U.S. Ser. No. 60/486,500, filed Jul. 10, 2003.

### GOVERNMENT SUPPORT

The research leading to the present invention was supported, at least in part, by a grant from NIH (CA82885, CA91490 and CA097262). Accordingly, the Government may have certain rights in the invention.

### FIELD OF THE INVENTION

The invention is in the field of cancer therapeutics. More specifically, the invention relates to compositions and methods for treating, and monitoring the treatment of, cancers, including fibrosarcomas and pancreatic cancers.

### 1. BACKGROUND OF THE INVENTION

Cancer, the uncontrolled growth of malignant cells, is a major health problem of the modern medical era. One-third of all individuals in the United States alone will develop cancer. Although the five-year survival rate has risen dramatically, i.e., by nearly fifty percent as a result of progress in early diagnosis and therapy, cancer still remains second only to cardiac disease as a cause of death in the United States. Indeed, some twenty percent of Americans will die from cancer, half due to lung, breast, and colon-rectal cancer. Moreover, skin cancer remains a major health hazard. Despite advances in prevention and early detection, refinements in surgical technique, and improvements in adjuvant radiotherapy and chemotherapy, the ability to cure many patients of cancer remains elusive. This is especially true in the case of pancreatic cancer, which remains one of the most deadly forms of the disease.

The American Cancer Society estimates that some 30,700 Americans (14,900 men and 15,800 women) will be diagnosed with cancer of the pancreas during 2003. An estimated 30,000 Americans (14,700 men and 15,300 women) will die of pancreatic cancer in 2003, making this type of cancer the fourth leading cause of cancer death in men and in women. Only about 21% of patients with cancer of the exocrine pancreas survive at least one year after diagnosis, with about 5% surviving five years after diagnosis. Only about 10% of cancers of the pancreas appear to be contained entirely within the pancreas at the time they are diagnosed. Attempts to remove the entire cancer by surgery may be successful in some of these patients. But, even when no spread beyond the pancreas is apparent at the time of surgery, a small number of cancer cells may already have spread to other parts of the body but have not formed tumors large enough to be detected in their new location. Even for those people diagnosed with local-stage disease the 5-year survival rate (i.e., the percentage of patients who live at least 5 years after their cancer is diagnosed) is only 17%.

There are, currently, essentially no useful treatments to extend the life of such patients. Indeed the National Cancer Institute (NCI) advisory board recently pointed this out and urged the NCI to give a high priority to research on this form of cancer. As underscored by the National Institutes of Health (NIH) in 2002, new strategies for treating pancreatic cancer are badly needed.

### 2. SUMMARY OF THE INVENTION

It has been discovered that patient fibrosarcomas and human fibroblast cell lines express 8-18-fold higher levels of the transcription factor Sp1 than the normal (i.e., noncancerous) cells from which they originated (see e.g., Example 5.4). Based in part on this and related findings disclosed herein, the invention provides novel methods and compositions for treating cancers, and particularly those cancers, such as pancreatic cancer and fibrosarcomas, associated with elevated levels of expression of Sp1 and/or Sp3.

Non-specifically, the invention provides methods and compositions for treating a cancerous condition, e.g., by reducing the rate of growth of a tumor, using one or more inhibitory agents that target the transcription factors Sp1 or Sp3. Compositions provided include Sp1 and Sp3—targeted antisense oligonucleotides ribozymes and recombinant viral vectors for delivery of these ribozymes to a tumor, as well as Sp1/Sp3 specific promoters for optimized negative-feedback expression of the Sp1/Sp3 ribozymes. Other Sp1/Sp3 inhibitor agents include Sp1/Sp3 binding-site triplex-forming oligonucleotides Sp1/Sp3 transcription factor binding site decoy oligonucleotides, agents targeting the Sp1 or Sp3 polypeptide, Sp1- and Sp3-binding aptamers, and small molecule antagonists. The invention further provides a system for analyzing the effectiveness of a cancer therapy treatment on a cancer, e.g., a pancreatic carcinoma tumor, by using an animal model in which the cancerous cells and recombinant cancer therapeutics are separately marked with different fluorescent markers that allow for the detection of effectively treated cancer cells and tissue.

In one aspect, the invention provides a method of inhibiting the growth of a cancerous cell that expresses an Sp1 and/or Sp3 transcription factor by contacting the cell with an Sp1/3-inhibitory agent so as to decrease the level of Sp1/Sp3, thereby inhibiting the growth of the Sp1/Sp3-expressing cancerous cell. In certain embodiments, the Sp1/Sp3-inhibitory agent is an Sp1-targeted ribozyme, an Sp1-targeted antisense oligonucleotide, or an Sp1-targeted siRNA. In other embodiments, the Sp1/Sp3-inhibitory agent is an Sp3-targeted ribozyme, an Sp3-targeted antisense oligonucleotide, or an Sp3-targeted siRNA. In other embodiments, the Sp1/Sp3-inhibitory agent is an Sp1 transcription factor decoy oligonucleotide, an Sp1 site triplex-forming oligonucleotide or an Sp1-binding aptamer.

In still other embodiments, the Sp1/Sp3-inhibitory agent is an Sp3 transcription factor decoy oligonucleotide, an Sp3 site triplex-forming oligonucleotide, or an Sp3-binding aptamer. The Sp1/Sp3-inhibitory agent may also be an Sp1-targeting proteolytic agent, an Sp1 dominant negative molecule, or an Sp1-binding small molecule, or an Sp3-targeting proteolytic agent, Sp3 dominant negative molecule, or Sp3-binding small molecule.

In certain particularly useful embodiments, the Sp1/Sp3-inhibitory agent, e.g., ribozyme or interfering RNA antisense RNA, is expressed from an Sp1/Sp3-autoregulatory expression system such as the Sp1 gene promoter sequence or an Sp1/Sp3 binding site.

In another aspect, the invention provides a method of treating or preventing a cancerous growth or condition associated with Sp1/Sp3 overexpression in a subject by administering an Sp1/Sp3-inhibitory agent to the subject so as to decrease the level of Sp1/Sp3 overexpression and thereby treat or prevent the cancerous growth or condition associated with Sp1/Sp3 overexpression in the subject. In certain embodiments the Sp1/Sp3-inhibitory agent is an Sp1-targeted ribozyme, an Sp3-targeted ribozyme, an Sp1-targeted antisense oligonucleotide, an Sp3-targeted antisense oligonucleotide, an Sp1-targeted siRNA, an Sp3-targeted siRNA, an Sp1-targeting proteolytic agent, an Sp3-targeting proteolytic agent, an Sp1 dominant negative molecule, an Sp3 dominant negative molecule, an Sp1-binding aptamer, an Sp3-binding aptamer, an Sp1-binding small molecule, an Sp3-binding small molecule, an Sp1/Sp3 transcription factor decoy oligonucleotide, or an Sp1/3 site triplex-forming oligonucleotide. In a particular embodiment, the Sp1/Sp3-inhibitory agent is expressed from an Sp1/Sp3-autoregulatory expression system such as an Sp1 gene promoter sequence or an Sp1/Sp3 binding site.

In another aspect, the invention provides nucleic acids for expression of a tumor cell therapeutic agent in an Sp1/Sp3 responsive-manner using an Sp1/Sp3-responsive expression system for expressing a sequence encoding the tumor cell therapeutic agent. In one embodiment, the tumor cell therapeutic agent is an Sp1/Sp3-inhibitory agent, but delivery of other cancer therapeutic agents is also contemplated. In another embodiment, the Sp1/Sp3-inhibitory agent is expressed from the Sp1/Sp3-responsive expression system in an auto-regulatory manner.

In certain embodiments of this aspect of the invention, the Sp1/Sp3-inhibitory agent is an Sp1-targeted ribozyme, an Sp3-targeted ribozyme, an Sp1-targeted antisense oligonucleotide, an Sp3-targeted antisense oligonucleotide, an Sp1-targeted siRNA, an Sp3-targeted siRNA, an Sp1-targeting proteolytic agent, an Sp3-targeting proteolytic agent an Sp1-targeted dominant negative agent, or an Sp3-targeted dominant negative agent. In a particular embodiment, the Sp1/Sp3-responsive expression system is an Sp1 gene promoter sequence or a naturally occurring or synthetic Sp1/Sp3-responsive expression system containing multiple Sp1/Sp3-binding sites (e.g., the sequence GGGCGG or GGGGCGGGG). In some embodiments, the Sp1/Sp3-responsive expression system or other Sp1/Sp3-inhibitory agent deliver system is a vector nucleic acid sequence, such as an adenoma-associated virus vector sequence.

In another aspect, the invention provides a method of detecting the efficaciousness of treatment, in certain instances with a recombinant therapeutic agent, of tumor cells in situ. In this aspect of the invention, a nucleic acid that encodes a tumor cell therapeutic agent and further expresses a fluorescent protein is administered to a mammalian organism that expresses a different fluorescent protein from a tumor or other cancerous cell. According to this aspect of the invention, co-expression of the first (i.e., tumor cell-expressing) and the second (i.e., recombinant therapeutic agent) fluorescent proteins, indicates that the tumor cell therapeutic agent has effectively treated the tumor or other cancerous cell. In certain embodiments, the fluorescent proteins used are green fluorescent protein and red fluorescent protein, and co-expression is detected as yellow fluorescence.

In yet another aspect, the invention provides a method of detecting in situ the treatment of a tumor or cancerous growth associated with Sp1/Sp3 overexpression in a mammalian organism using a mammalian organism expressing a first fluorescent protein in a cancerous cell that is overexpressing Sp1 and/or Sp3. According to this aspect of the invention, the mammalian organism expresses a first fluorescent protein in a tumor or other cancerous cell and is administered a nucleic acid that encodes an Sp1/Sp3-inhibitory agent and also expresses a second fluorescent protein. The co-expression of the first and the second fluorescent protein is then examined. Co-expression of the first and the second fluorescent protein is indicative of effective treatment of the cancerous cells by the Sp1/Sp3-inhibitory agent. In certain embodiments, the fluorescent proteins used are green fluorescent protein and red fluorescent protein, and co-expression is detected as yellow fluorescence. In particular embodiments, the Sp1/Sp3-inhibitory agent is an Sp1-targeted ribozyme, an Sp3-targeted ribozyme, an Sp1-targeted antisense oligonucleotide, an Sp3-targeted antisense oligonucleotide, an Sp1-targeted siRNA, an Sp3-targeted siRNA, an Sp1-targeting proteolytic agent, an Sp3-targeting proteolytic agent, an Sp1/Sp3-binding site oligonucleotide, or an Sp1/Sp3-competitive binding agent. In one embodiment the Sp1/Sp3-inhibitory agent is expressed from an autoregulatory Sp1/Sp3-responsive expression system.

In another aspect, the invention provides a method of treating a pancreatic or fibroblast cancerous growth associated with Sp1/Sp3-overexpression by administering an Sp1-inhibitory agent or an Sp3-inhibitory agent so as to decrease the level of Sp1 and/or Sp3, thereby inhibiting the growth of the Sp1/Sp3-overexpressing cancerous cell. In some embodiments, particular Sp1/Sp3-inhibitory agents, such as an Sp1-targeted ribozyme or an Sp3-targeted ribozyme, are used. The Sp1-targeted ribozyme or Sp3-targeted ribozyme are expressed in an Sp1/Sp3-responsive manner from an Sp1/Sp3-responsive expression system such as an Sp1 gene promoter sequence or other natural or synthetic promoter sequence containing a plurality of G/C-box Sp1/Sp3-binding sites (e.g., the sequence: GGGGCGGGG).

### 3. BRIEF DESCRIPTION OF THE FIGURES

FIG. 1 is a diagramatic representation showing the generation of the MSU lineage of human fibroblasts.

FIG. 2A is a representation of a Western blot showing Sp1 expression in human fibroblasts including: normal human fibroblasts (lanes 1-5) and human fibrosarcoma-derived cell lines (lanes 6-10).

FIG. 2B is a representation of a Western blot showing Sp1 expression in human fibroblasts including: LG1 (lane 5); MSU-1.0 (lane 11); MSU-1.I (lane 12); and carcinogen-transformed MSU-1. 1 cells) (lanes 13-19).

FIG. 3A is a representation of the results of an electrophoretic mobility shift assay showing Sp1 binding activity in nuclear extracts from normal and malignantly transformed human fibroblasts including: normal human fibroblasts (lanes 1-4); LGI (lane 5); and tumor (fibrosarcoma) cell lines (lanes 6-10).

FIG. 3B is a representation of the results at an electrophoretic mobility shift assay showing Sp1 binding activity in nuclear extracts from normal and malignantly transformed human fibroblasts including: LGI (lane 5); MSU-1.0 (lane 1); MSU-1.1 (lane 12); and and tumor (fibrosarcoma) cell lines (lanes 6-10).

FIG. 4A is a schematic representation shows the sequence and schematic representation of an Sp1 targeted ribozyme wherein the arrow indicates the cleavage site on the targeted mRNA, and the position of the complementary sequences targeted in full-length Sp1 nucleic acid sequence is indicated by the shaded region shown in FIG. 11(A).

FIG. 4B is a schematic representation shows the Sp3-complementary sequence of an Sp3 targeted ribozyme (wherein the arrow indicates the cleavage site on the targeted mRNA, and the position of the complementary sequences targeted in full-length Sp3 nucleic acid sequence is indicated by the shaded and region shown in FIG. 11(C)).

FIG. 5A is a representation of a Western blot showing that levels of Sp1 (top panel) and Sp3 (bottom panel) protein are specifically depleted by an Sp1 hammerhead ribozyme in a PH3MT cell line.

FIG. 5B is a representation of a Western blot showing that levels of Sp1 (top panel) and Sp3 (bottom panel) protein are specifically depleted by an Sp1 hammerhead ribozyme in a MW7.3A2/SB1 cell line.

FIG. 6A is representation of a Western blot showing that levels of Sp3 protein are specifically depleted by Sp3 hammerhead ribozyme.

FIG. 6B is a representation of a Western blot showing that levels of Sp1 protein are specifically depleted by Sp3 hammerhead ribozyme

FIG. 6C is a representation of a Western blot showing that levels of actin protein are not affect by the Sp3 hammerhead ribozyme

FIG. 7 is a representation of a Western blot demonstrating that levels of Sp1 are depleted in Sp1 ribozyme treated pancreatic carcinoma cell lines.

FIG. 8 is a diagrammatic representation showing the construction of pAAV vectors for expressing Sp1 or Sp3 ribozymes and/or fluorescent protein markers.

FIG. 9 is a diagrammatic representation showing the structure of Sp1/Sp3-specific promoters.

FIG. 10A is a schematic representation showing the structure of rAAV PURO-Sp1/Sp3 constructs carrying the PURO sequence and an Sp1 or Sp3 ribozyme-encoding sequence.

FIG. 10B is a schematic representation showing the structure of rAAV EGFP-Sp1/Sp3 constructs carrying the EGFP sequence and an Sp1 or Sp3 ribozyme-encoding sequence.

FIG. 10C is a schematic representation showing the structure of rAAV PURO-EGFP constructs carrying the PURO sequence and the EGFP sequence.

FIG. 11A is a schematic representation showing the nucleic acid sequence of a human SP1 (i.e., GenBank Accession No. XM_{—}028606; SEQ ID NO:1; the position of the complementary sequence targeted by the hammerhead ribozyme shown in FIG. 4A is indicated by the shaded region and the underlined and bolded “C” in this region is the point at which the ribozyme is predicted to catalyze 3′ cleavage).

FIG. 11B is a schematic representation of the polypeptide sequence of a human SP1 (i.e., GenBank Accession No. XP_{—}028606; SEQ ID NO:2).

FIG. 11C is a schematic representation showing the nucleic acid sequence of a human SP3 (i.e., GenBank Accession No. XM_{—}092672; SEQ ID NO:3; the position of the complementary sequence targeted by the hammerhead ribozyme shown in FIG. 4B is indicated by the shaded region and the underlined and bolded “C” in this region is the point at which the ribozyme is predicted to catalyze 3′ cleavage).

FIG. 11D is a schematic representation of the polypeptide sequence of a human SP3 (i.e., GenBank Accession No. XP_{—}092672; SEQ ID NO:4).

FIG. 12A is a representation of a Western blot showing Sp1 protein expression in whole cell lysates of the foreskin-derived normal human fibroblast cell line (LG1), its non-transformed, infinite life span derivative cell strain (MSU-1.1), and a corresponding malignant cell line (PH2MT) derived from a tumor formed in an athymic mouse by MSU-1.1 cells that had been transfected with H-ras oncogene. The bottom panel is a representation of a Western blot showing β-actin protein levels (as a loading control).

FIG. 12B is a representation of a Western blot showing Sp1 protein expression in whole cell lysates of the foreskin-derived normal human fibroblast cell line (LG1), its non-transformed, infinite life span derivative cell strain (MSU-1.1), and malignant cell line y2-3A/SB1, derived from a tumor formed in athymic mice by MSU-1.1 cells that had been transformed by exposure to ⁶⁰Cobalt radiation.

FIG. 12C is a schematic representation showing the sequence and structure of the Sp1 U1snRNA/Ribozyme construct used to reduce expression of Sp1. The antisense sequence is complementary to the 165-205 sequence of human Sp1 mRNA (Genbank No. AJ272134) and contains a hammerhead ribozyme sequence in its center, flanked by the U1snRNA. Cleavage is predicted to occur at nucleotide 185, 5′ to the sequence GUC (arrow).

FIG. 12D is a schematic representation showing the sequence and structure of the Sp1 U1snRNA/ribozyme predicted by the Mulfold and Loop-D-Loop programs.

FIG. 13A is a representation of a Western blot showing Sp1 and Sp3 protein expression in whole cell lysates of tumor-derived cell line PH2MT (lane 1), two derivative cell strains transfected with the vector alone as controls (lanes 2 and 3), and two Sp1-ribozyme-transfected derivative strains (lanes 4 and 5).

FIG. 13B is a representation of a Western blot showing Sp1 and Sp3 protein expression in whole cell lysates of tumor-derived cell line γ2-3A/SB1 (lane 1), the same with vector alone (lanes 2 and 3), and with the Sp1 U1snRNA/Ribozyme (lanes 4 -6).

FIG. 13C is a graphical representation showing the Sp1/Sp3 transactivation activity in parental cell line PH2MT (column 1), two vector-transfected derivative cell strains (columns 2 and 3), and two Sp1-U1snRNA/Ribozyme-transfected cell strains (columns 4 and 5).

FIG. 13D is a graphical representation showing the Sp1/Sp3 transactivation activity parental cell line γ2-3A/SB1 (column 1), two vector-transfected cell strains (columns 2 and 3), and three Sp1 U1snRNA/Ribozyme-transfected cell strains (columns 4-6).

FIG. 13E is a photographic representation of the results of RT-PCR analysis of the level of Sp1 mRNA in parental cell line PH2MT (lane 1), its two vector transfectants (lanes 2 and 3), and the two Sp1 U1snRNA/Ribozyme transfectants (lanes 4 and 5).

FIG. 13F is a photographic representation of the results of RT-PCR analysis of the level of Sp1 mRNA in cell line γ2-3A/SB1 (lane 1), with the vector control cell lines (lanes 2 and 3) and in those for the Sp1 U1snRNA/Ribozyme transfectants in lanes 4-6. The cell strain in lane 5 showed a decreased level of Sp1 mRNA.

FIG. 14A is a graphical representation of colony forming ability of cell lines and strains showing that decreased expression of Sp1 protein inhibits anchorage-independent growth of the malignant cell lines and strains. The graph depicts an average number of colonies with diameters of designated size calculated and plotted as percent of the total (V, empty vector control; SpR, transfectants expressing Sp1 ribozyme).

FIG. 14B is a graphical representation of colony forming ability of cell lines and strains, substantially as shown above in FIG. 14A, except identical experiments carried out with a second series of cell lines/strains using derivatives of cell line γ2-3A/SB1. The error bars show the standard error of the mean.

FIG. 15A is a photographic representation of the morphology of the malignant PH2MT cell line.

FIG. 15B is a photographic representation of the control PH2MT transfected with an empty vector.

FIG. 15C is a photographic representation of the control PH2MT transfected with an empty vector.

FIG. 15D is a photographic representation of the control PH2MT transfected with the Sp1 U1snRNA/ribozyme showing that down-regulation of Sp1 protein level changes cell morphology.

FIG. 15E is a photographic representation of the control PH2MT transfected with the Sp1 U1snRNA/ribozyme showing that down-regulation of Sp1 protein level changes cell morphology.

FIG. 15F is a photographic representation of the morphology of the γ2-3A/SB1 cells.

FIG. 15G is a photographic representation of the morphology of the γ2-3A/SB1 cells transfected with an empty vector.

FIG. 15H is a photographic representation of the morphology of the γ2-3A/SB1 cells transfected with an empty vector.

FIG. 15I is a photographic representation of the morphology of the γ2-3A/SB1 cells transfected with the Sp1 U1snRNA/ribozyme showing that down-regulation of Sp1 protein level changes cell morphology.

FIG. 15J is a photographic representation of the morphology of the γ2-3A/SB1 cells transfected with the Sp1 U1snRNA/ribozyme showing that down-regulation of Sp1 protein level changes cell morphology.

FIG. 15K is a representation of a control Western Blot showing beta-actin and Ku80 protein in the cells shown in FIGS. 15A-15E (lanes 1-5).

FIG. 15L is a representation of a control Western Blot showing beta-actin and Ku80 protein in the cells shown in FIGS. 15F-15J (lanes 1-5).

FIG. 16A is a graphical representation of the percentage of floating cells in PH2MT cells, as well as the corresponding vector-treated (V1 and V2) and Sp1-U1snRNA/Ribozyme-transfected cells (SpR2 and SpR3) showing that down-regulation of Sp1 expression induces apoptosis.

FIG. 16B is a graphical representation of the percentage of floating cells in y2-3A/SB1 cells, as well as the corresponding vector-treated (V1 and V2) and Sp1-U1snRNA/Ribozyme-transfected cells (SpR2 and SpR3) showing that down-regulation of Sp1 expression induces apoptosis.

FIG. 16C is a graphical representation of the result of 3-4 independent experiments assessing apoptosis of PH2MT cells, as well as the corresponding vector-treated (V1 and V2) and Sp1-U1snRNA/Ribozyme-transfected cells (SpR2 and SpR3), using flow cytometry.

FIG. 16D is a graphical representation of the result of 3-4 independent experiments assessing apoptosis of γ2-3A/SB1 cells, as well as the corresponding vector-treated (V1 and V2) and Sp1-U1snRNA/Ribozyme-transfected cells (SpR2 and SpR3), using flow cytometry.

FIG. 17A is a representation of a Western blot showing the expression of MET, EGFR and uPAR detected using anti-MET, anti-EGFR and anti-uPAR antibodies in whole cell lysates prepared from PH2MT cells (lane 1), vector control cell strains (lanes 2 and 3), and Sp1-U1snRNA/Ribozyme-transfected cell strains (lanes 4 and 5), with Ku80 served as loading control.

FIG. 17B is a representation of a Western blot showing the expression of MET, EGFR and uPAR detected using anti-MET, anti-EGFR and anti-uPAR antibodies in whole cell lysates prepared from γ2-3A/SB1 cells (lane 1), vector control cell strains (lanes 2 and 3), and Sp1-U1snRNA/Ribozyme-transfected cell strains (lanes 4 and 5).

FIG. 17C is a representation of a Western blot showing the expression of HGF and uPA in whole cell lysates were prepared from PH2MT cells (lane 1), vector control cell strains (lanes 2 and 3), and Sp1-U1snRNA/Ribozyme-transfected cell strains (lanes 4 and 5).

FIG. 17D is a representation of a Western blot showing the expression of HGF and uPA in whole cell lysates were prepared from γ2-3A/SB1 cells (lane 1), vector control cell strains (lanes 2 and 3), and Sp1-U1snRNA/Ribozyme-transfected cell strains (lanes 4 and 5).

FIG. 17E is a graphical representation of the levels of the level of expression of VEGF determined by ELISA in PH2MT cells (lane 1), and the corresponding vector control cell strains (lanes 2 and 3), and Sp1-U1snRNA/Ribozyme-transfected cell strains (lanes 4 and 5).

FIG. 17F is a graphical representation of the levels of the level of expression of VEGF determined by ELISA in γ2-3A/SB1 cells (lane 1), and the corresponding vector control cell strains (lanes 2 and 3), and Sp1-U1snRNA/Ribozyme-transfected cell strains (lanes 4 and 5).

FIG. 18 is a representation of a Western blot analysis of Sp1 expression in SHAC cells showing vector controls (V1, V2), and SpR transfectants (SpR1, SpR2, SpR3) expressing the Sp1U1snRNA/Ribozyme.

### 4. DETAILED DESCRIPTION OF THE INVENTION

4.1. General

The present invention is based, in part, upon the observation that overexpression of Sp1 (and Sp3) plays a causal role in the tumorigenicity. In particular, patient fibrosarcomas, as well as human fibroblast cell lines that have been transformed into malignant cells in culture, express 8- to 18-fold higher levels of transcription factor Sp1 than their normal parental cells (as well as elevated Sp3 levels). Further, when these Sp1/Sp3-overexpressing cell lines are injected subcutaneously into athymic mice, tumors form rapidly and these tumors express the same high level of Sp1 (and Sp3) as the injected cells. Additionally, transfection of fibrosarcoma-derived Sp1-overexpressing cell lines with Sp1-specific ribozymes was found to cause a coupled decrease in Sp1 protein levels and tumor forming ability, and, Sp3 ribozyme-transfectants also exhibited a coupled decrease in Sp3 expression and tumorigenicity.

Thus, the invention provides compositions and methods of inhibiting the growth of an Sp1/Sp3-expressing cancerous cell with an Sp1/3-inhibitory agent. The Sp1/Sp3-inhibitory agent may be any agent capable of reducing intracellular levels of Sp1 and/or Sp3 including agents that inhibit or reduce expression of the Sp1 and/or Sp3 gene; agents that reduce levels of Sp1 and/or Sp3 messenger RNA (i.e., mRNA); agents that block translation of Sp1 and/or Sp3 messenger RNAs; and agents that target Sp1 and/or Sp3 for degradation (e.g., by targeting Sp1 and/or Sp3 for proteolytic degradation).

While the instant invention provides support for the general concept that if Sp1 and/or Sp3 levels are elevated in cancerous cells, that inhibition of either the Sp1 or Sp3 may be used to inhibit the resulting cancerous growth, the invention is not limited so as to preclude therapeutics and therapeutic methods targeting only Sp1 or only Sp3 in instances where only one or the other factor is present in elevated levels in the cancerous cell as would be readily determinable for a given form of cancer by the person of skill in the art. Accordingly, the Sp1/3-inhibitory agents of the invention include agents capable of decreasing levels of either Sp1 or Sp3 (or both) gene expression, mRNA levels, protein levels or protein activity. In particular, while the instant invention provides support for the general concept that targeting either Sp1 or Sp3 for inhibition results in decreased expression of both by virtue of their own Sp1/Sp3-dependent promoters, the invention is not limited to this mechanism of action. In particular, the invention includes independent aspects encompassing Sp1 and Sp3 inhibitory agents regardless of each agent's inhibitory effect on the other Sp family member gene expression, mRNA levels, protein levels and/or protein activity. For example, the term “Sp1/Sp3-inhibitory agent” encompasses, e.g., Sp1 ribozymes and methods of their use regardless of their effect (or lack thereof) on Sp3 gene expression, Sp3 mRNA levels, Sp3 protein levels or Sp3 protein activity.

4.2 Definitions

As used herein, the following terms and phrases shall have the meanings set forth below. Unless defined otherwise, all technical and scientific terms used herein have the same meaning as commonly understood to one of ordinary skill in the art to which this invention belongs.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or noncovalently linked to form antibodies having two or more binding sites. The subject invention includes polyclonal, monoclonal, or other purified preparations of antibodies and recombinant antibodies.

The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, tumors, sputum, blood, blood cells (e.g., white cells), tissue or fine needle biopsy samples, urine, peritoneal fluid, and pleural fluid, or cells there from. Biological samples may also include sections of tissues such as frozen sections taken for histological purposes.

The term “biomarker” of a disease refers to a gene which is up- or down-regulated in a diseased cell of a subject having the disease relative to a counterpart normal cell, which gene is sufficiently specific to the diseased cell that it can be used, optionally with other genes, to identify or detect the disease. Generally, a biomarker is a gene that is characteristic of the disease (e.g., Sp1 or Sp3 in the case of a pancreatic carcinoma).

A nucleotide sequence is “complementary” to another nucleotide sequence if each of the bases of the two sequences matches, i.e., are capable of forming Watson Crick base pairs. The term “complementary strand” is used herein interchangeably with the term “complement.” The complement of a nucleic acid strand can be the complement of a coding strand or the complement of a non-coding strand.

The phrases “conserved residue” “or conservative amino acid substitution” refer to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include:
(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Tip,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.
   In addition to the groups presented above, each amino acid residue may form its own group, and the group formed by an individual amino acid may be referred to simply by the one and/or three letter abbreviation for that amino acid commonly used in the art.

The term “interact” as used herein is meant to include detectable relationships or association (e.g., biochemical interactions) between molecules, such as interaction between protein-protein, protein-nucleic acid, nucleic acid-nucleic acid, and protein-small molecule or nucleic acid-small molecule in nature.

The term “interacting protein” refers to protein capable of interacting, binding, and/or otherwise associating to a protein of interest, such as for example a human Sp1 protein.

The term “isolated” as used herein with respect to nucleic acids, such as DNA or RNA, refers to molecules separated from other DNAs, or RNAs, respectively that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized.

Moreover, an “isolated nucleic acid” is meant to include nucleic acid fragments, which are not naturally occurring as fragments and would not be found in the natural state. The term “isolated” is also used herein to refer to polypeptides, which are isolated from other cellular proteins and is meant to encompass both purified and recombinant polypeptides.

As used herein, the terms “label” and “detectable label” refer to a molecule capable of detection, including, but not limited to, radioactive isotopes, fluorophores, chemiluminescent moieties, enzymes, enzyme substrates, enzyme cofactors, enzyme inhibitors, dyes, metal ions, ligands (e.g., biotin or haptens) and the like. The term “fluorescer” refers to a substance or a portion thereof, which is capable of exhibiting fluorescence in the detectable range. Particular examples of labels which may be used under the invention include fluorescein, rhodamine, dansyl, umbelliferone, Texas red, luminol, NADPH, alpha-beta-galactosidase and horseradish peroxidase.

The “level of expression of a gene in a cell” refers to the level of mRNA, as well as pre-mRNA nascent transcript(s), transcript processing intermediates, mature mRNA(s) and degradation products, encoded by the gene in the cell.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides, ESTs, chromosomes, cDNAs, mRNAs, and rRNAs are representative examples of molecules that may be referred to as nucleic acids.

The term “oligonucleotide” refers to an oligomer or polymer of nucleotide or nucleoside monomers consisting of naturally occurring bases, sugars and intersugar (backbone) linkages. The term also includes modified or substituted oligomers comprising non-naturally occurring monomers or portions thereof, which function similarly. Incorporation of substituted oligomers is based on factors including enhanced cellular uptake, or increased nuclease resistance and are chosen as is known in the art. The entire oligonucleotide or only portions thereof may contain the substituted oligomers.

The term “percent identical” refers to sequence identity between two amino acid sequences or between two nucleotide sequences. Identity can each be determined by comparing a position in each sequence, which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including Hidden Markov Model (HMM), FASTA and BLAST. HMM, FASTA and BLAST are available through the National Center for Biotechnology Information, National Library of Medicine, National Institutes of Health, Bethesda, Md. and the European Bioinformatic Institute EBI. In one embodiment, the percent identity of two sequences that can be determined by these GCG programs with a gap weight of 1, e.g., each amino acid gap is weighted as if it were a single amino acid or nucleotide mismatch between the two sequences. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Where desirable, an alignment program that permits gaps in the sequence is utilized to align the sequences. The Smith Waterman is one type of algorithm that permits gaps in sequence alignments (see (1997) Meth. Mol. Biol. 70: 173-187). Also, the GAP program using the Needleman and Wunsch alignment method can be utilized to align sequences. More techniques and algorithms including use of the HMM are described in Sequence, Structure, and Databanks: A Practical Approach (2000), ed. Oxford University Press, Incorporated. In Bioinformatics: Databases and Systems (1999) ed. Kluwer Academic Publishers. An alternative search strategy uses MPSRCH software, which runs on a MASPAR computer. MPSRCH uses a Smith-Watermnan algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases. Databases with individual sequences are described in Methods in Enzymology, ed. Doolittle, supra. Databases include Genbank, EMBL, and DNA Database of Japan (DDBJ).

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. A mismatch in a duplex between a target polynucleotide and an oligonucleotide or polynucleotide means that a pair of nucleotides in the duplex fails to undergo Watson-Crick bonding. In reference to a triplex, the term means that the triplex consists of a perfectly matched duplex and a third strand in which every nucleotide undergoes Hoogsteen or reverse Hoogsteen association with a base pair of the perfectly matched duplex.

The term “RNA interference,” “RNAi,” or “siRNa” all refer to any method by which expression of a gene or gene product is decreased by introducing into a target cell one or more double-stranded RNAs, which are homologous to the gene of interest (particularly to the messenger RNA of the gene of interest, e.g., Sp1 or Sp3).

Polymorphic variants also may encompass “single nucleotide polymorphisms” (SNPs) in which the polynucleotide sequence varies by one base (e.g., a one base variation in Sp1 or Sp3). The presence of SNPs may be indicative of, for example, a certain population, a disease state, or a propensity for a disease state.

The “profile” of an aberrant, e.g., tumor cell's biological state refers to the levels of various constituents of a cell that change in response to the disease state. Constituents of a cell include levels of RNA, levels of protein abundances, or protein activity levels.

The term “protein” is used interchangeably herein with the terms “peptide” and “polypeptide.”

The term “recombinant protein” refers to a protein of the present invention which is produced by recombinant DNA techniques, wherein generally DNA encoding the expressed protein or RNA is inserted into a suitable expression vector which is in turn used to transform a host cell to produce the heterologous protein or RNA. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations, including substitutions and deletions, of a naturally occurring protein.

The term “Sp1/Sp3-expressing cancerous cell,” as used herein means a cancerously transformed cell that is expressing higher levels of either Sp1 or Sp3 transcription factor as compared to the normal cell from which it derived. While the instant invention provides support for the general concept that both Sp1 and Sp3 levels are elevated in some cancerous cells and, if so, that inhibition of either the Sp1 or Sp3 may be used to inhibit the cancerous growth, the invention is not limited so as to preclude therapeutics and therapeutic methods targeting only Sp1 or only Sp3 in instances where only one or the other factor is present in elevated levels in the cancerous cell as would be readily determinable for a given form of cancer by the person of skill in the art.

Similarly, the term “Sp1/Sp3-inhibitory agent,” as used herein means an agent capable of decreasing levels of either Sp1 or Sp3 (or both) gene expression, mRNA levels, protein levels or protein activity. In particular, while the instant invention provides support for the general concept that targeting either Sp1 or Sp3 for inhibition results in decreased expression of both by virtue of their own Sp1/Sp3-dependent promoters, the invention is not limited to this mechanism of action. In particular, the invention includes independent aspects encompassing Sp1 and Sp3 inhibitory agents regardless of each agent's inhibitory effect on the other Sp family member gene expression, mRNA levels, protein levels and/or protein activity. Accordingly, the term “Sp1/Sp3-inhibitory agent” encompasses, e.g., Sp1 ribozymes and methods of their use regardless of their effect (or lack thereof) on Sp3 gene expression, Sp3 mRNA levels, Sp3 protein levels or Sp3 protein activity.

As used herein, the term “transfection” means the introduction of a nucleic acid, e.g., via an expression vector, into a recipient cell by nucleic acid-mediated gene transfer.

“Transformation”, other than when used to refer to cancerous or oncogenic transformation, refers to a process in which a cell's genotype is changed as a result of the cellular uptake of exogenous DNA or RNA, and, for example, the transformed cell expresses a recombinant form of an RNA or polypeptide or, in the case of anti-sense expression from the transferred gene, the expression of a naturally-occurring form of the polypeptide is disrupted.

As used herein, the term “transgene” means a nucleic acid sequence (encoding, e.g., one of the target nucleic acids, or an antisense transcript thereto), which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

The term “treating” a disease in a subject or “treating” a subject having a disease refers to subjecting the subject to a pharmaceutical treatment, e.g., the administration of a drug, such that at least one symptom of the disease is decreased. Accordingly, the term “treating” as used herein is intended to encompass curing as well as ameliorating at least one symptom of the condition or disease.

A “variant” of polypeptide X refers to a polypeptide having the amino acid sequence of peptide X in which is altered in one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR).

The term “variant,” when used in the context of a polynucleotide sequence, may encompass a polynucleotide sequence related to that of gene or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternative splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species.

The term “vector” refers to a nucleic acid molecule capable of transporting another nucleic acid to which it has been linked. One type of useful vector is an episome, i.e., a nucleic acid capable of extra-chromosomal replication. Useful vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

4.3 Sp1/Sp3 Nucleic Acids and Proteins

The invention provides Sp1 and Sp3 nucleic acids, proteins and related compositions, in particular, for use directly as, or in the production of, the Sp1/Sp3 inhibitory agents on the invention. The following information on Sp1 and Sp3 is provided as a guide for their use in the subject invention.

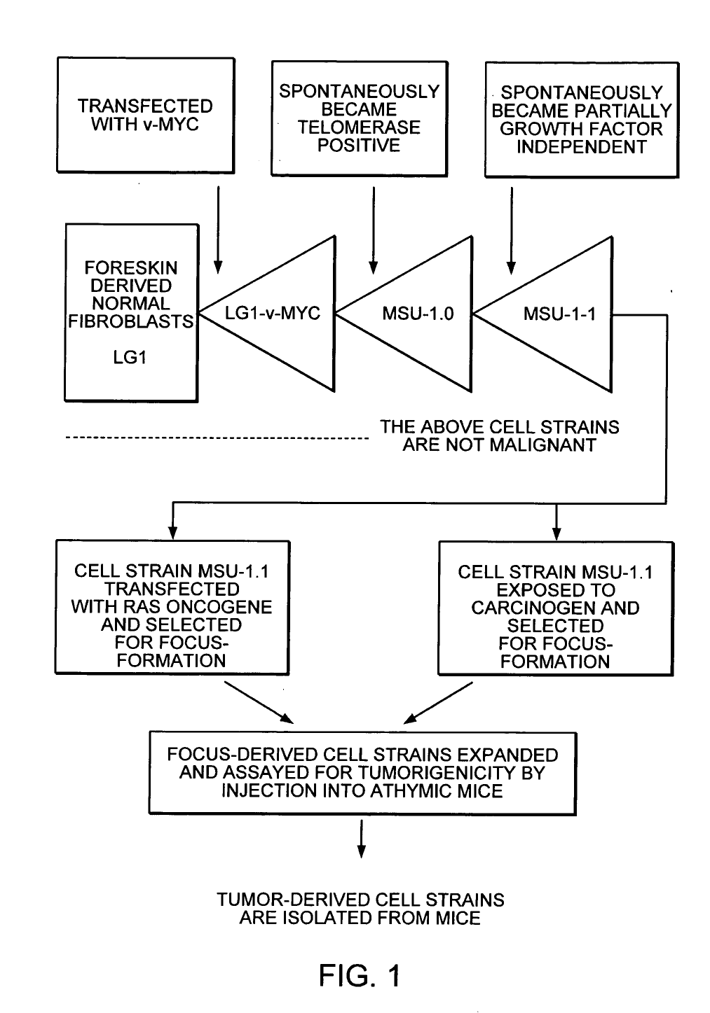

Sp1 was originally identified as the transcription factor that binds to and activates transcription from GC-boxes in the simian virus 40 (SV40), (see Dynan and Tjian (1983) Cell 32: 669-680, and Liang (1996) et al., Oncogene 13: 863-871), and cloned in 1987 by Kadonaga (see Kadonaga et al., (1987) Cell 51: 1079-1090). A partial 3′ clone of the human gene is described in GenBank Accession No. J03133; and mouse (GenBank Accession No. AF062566 and AF022363), and rat (GenBank Accession No. D12768) clones have also been described. FIGS. 1(A) and (B) show, respectively, the sequence of a full-length Sp1 coda (GenBank Accession No. XM_{—}028606; SEQ ID NO:1) and protein (GenBank Accession No. XP_{—}028606; SEQ ID NO:2). The human Sp1 gene is on chromosome 12 (at 12q13; see Gay nor et al., (1993) Cytokine. Cell Genet. 64: 210-2) and the mouse (at 15, see Safer et al., (1990) Genomics 8: 571-4) and the rat (at 7q36, see Choy et al., (1998) Cytokine. Cell Genet. 81: 273-4) genes have also been chromosomally localized. Sp1 was believed to be a basal transcription factor that functioned to keep a large number of housekeeping genes functioning at low levels, e.g., housekeeping genes lacking a TATE box or CAT box and have a single GC-rich promoter region containing one AGOG sequence, which is the binding site for Sp1 protein. It is generally agreed that Sp-factor binding to such GC-box sequences acts as an “on-off” switch for the gene linked to this sequence. It is now understood that important and widely distributed promoters contain G-rich elements such as the GC-box (GGGGCGGGG) as well as the related GT/CACCC-box (i.e., GGTGTGGGG), which occur in many ubiquitous as well as tissue-specific and viral genes.

Mapping of the Sp1 activation domain revealed that interspersed bulky hydrophobic amino acids are essential for transcriptional activation and not, per se, the glutamine residues (see Gill et al., (1994) Proc. Natl. Acad. Sci. (USA) 91: 192-6). An Sp1 inhibitory domain has been mapped to the N-terminus (see Murata et al., (1994) J. Biol. Chem. 269: 20674-20681). Synergistic activation of promoters by Sp1 through multiple GC-boxes requires in addition the short C-terminal domain D (see Pascal and Tjian (1991) Genes Dev. 5: 1646-56). Sp1 is known to be phosphorylated (Jackson et al., (1990) Cell 63: 155-65) and glycosylated (see Jackson and Tjian (1988), Cell 63: 155-65) and it is capable of forming homotypic interactions leading to multimeric complexes (see Mastrangelo (1991) Proc. Natl. Acad. Sci. (USA) 88: 5670-74; and Pascal (1991) Genes Dev. 5: 1626-56). In addition, many heterotypic interactions with different classes of nuclear proteins have been reported. These include factors belonging to the general transcription machinery, such as the TATA-box binding protein TBP, the TBP-associated factors dTAFII110/hTAFII130, and hTAFII55. Other proteins, which have been shown to interact with Sp1 are cell cycle regulators such as the retinoblastoma-related protein p1O7 and transcription factors such as YY1 or E2F. Sp1 can also bind to its target sequence in assembled nucleosomes, and it interacts with a large co-activator complex called CRSP (cofactor required for Sp1 activation) that stimulates Sp1-mediated transcription in vitro (see Ryu et al., (1999) Nature 397: 446-50).

Sp3, named because of its high homology to the Sp1 protein, was discovered in 1992 (see Hagen et al., (1992) Nucleic Acids Res. 20: 5519-5525) and occurs in three isoforms, i.e., a full-length 110-115 kDa form and two smaller isoforms (60-70 kDa), which arise from utilization of the first two internal AUG codons (see Kingsley and Winoto (1992) Mol. Cell Biol. 12: 4251-4261; and Kennett et al., (1997) Nucleic Acids. Res. 25: 3110-3117). Like Sp1, Sp3 protein is ubiquitously expressed and has a similar binding affinity to the GGGCGG sequence (see Kitadai et al., (1992) Biochem. Biophys. Res. Commun. 189: 1342-1348; Hagen et al., (1992) Nucleic Acids Res. 20: 5519-5525; Birnbaum et al., (1995) Biochemistry 34: 16503-16508; and Kumar and Butler (1997) Nucleic Acids Res. 25: 2012-2019). Clones of human Sp3 have been reported (see GenBank Accession Nos.: X68560 and S52144) and a full-length human Sp3 cDNA and corresponding encoded Sp3 polypeptide have been reported (see, respectively GenBank Accession Nos. XM_{—}092672 and XP_{—}092672 as shown in FIGS. 1(C) and (D)).

Both N-terminal glutamine-rich regions of Sp3 can act as strong activation domains on their own in both insect and in mammalian cells (see Majello (1994) Nucleic Acid Res. 22: 4914-21). The molecular basis for the inactivity of Sp3 under certain conditions has been mapped to an inhibitory domain located between the second glutamine-rich activation domain and the first zinc finger. The amino acid triplet KEE within this domain is absolutely essential for repressor function (see Dennig et al., (1996) EMBO J 15: 5659-67). Mutation of these amino acids to alanine residues converted almost inactive Sp3 to a strong activator. Significantly, the inhibitory domain of Sp3 acts as an independent module in cis. It can be transferred to other activation domains, which in turn lose their activation properties (see Dennig et al., supra). Purified recombinant Sp3 expressed in SL2 cells (see Braun and Suske (1999) Biotechniques 26(6): 1038-40) act in an in vitro system as strong activator similar to Sp1. Accordingly, additional proteins which act as co-repressors may be involved in the inhibitory function of this domain (e.g., a protein designated SIF-1 (Sp3-interacting protein 1) which specifically interacted with the wild type inhibitory domain but not with the mutated form).

Two other Sp protein family members, Sp2 and Sp4, are also known. Sp2 binds to a different DNA sequence than Sp1, Sp3, and Sp4. Sp4 binds to the same DNA sequence as Sp1 and Sp3, but is expressed predominantly in neurological tissue, particularly in the brain, but also in testis epithelial tissues and developing teeth (see Hagen et al., (1992) Nucleic Acids Res. 20: 5519-25; and Supp et al., (1996) Dev. Biol. 176: 284-99). Notably, disruption of this gene in mice results in behavioral abnormalities (see Supp et al., (1996) Dev. Biol. 176: 284-99).

All four human Sp-family members have similar domain structures, which include multiple amino-terminally situated activation domains as well as glutamine and serine/threonine-rich domains and a region that is rich in both basic and acidic charged amino acid residues. These transcriptional-regulatory regions are located upstream of the carboxy-terminally located DNA binding domain, which consists of three zinc finger DNA binding structures. The 81 amino acids C2H2-type zinc finger region, which represents the DNA-binding domain is the most highly conserved part of the proteins. Alignment of that region shows that Sp1, Sp3 and Sp4 are more closely related to each other than to Sp2. According to structural studies on zinc finger proteins bound to DNA (Fairall and Pavletich), one could predict that the amino acids KHA within the first, RER within the second and RHK within the third zinc finger contact specific bases. Significantly, these critical amino acids are all conserved in Sp1, Sp3 and Sp4, but not in Sp2. Consistently, Sp1, Sp3 and Sp4 recognize the classical Sp1-binding site with identical affinity (see Hagen et al., (1995) J. Biol. Chem. 270: 24989-994; and Hagen et al., (1992) Nucleic Acids Res. 20: 5519-25). In contrast, Sp2, in which the important histidine residue within the first zinc finger is replaced by a leucine residue does not bind to the GC-box but to a GT-rich element within the T-cell receptor gene 5′-flanking region (see Kingsley and Winoto (1992) Mol. Cell Biol. 12: 4251-61).

Many studies have reported that, whereas Sp1 up-regulates transcription, Sp3 functions to up- or down- regulate transcription. Recent studies (see Roder et al., (2002) Biochem. Biophys. Res. Commun. 293: 793-799 and Schafer et al., (2003) J. Biol. Chem. 278(10): 8190-8; and deGraffenried et al., (2002) J. Steroid Biochem. Mol. Biol. 82: 7-18) with transient co-transfection studies carried out in SL2 Drosophila cells show that expression of Sp3 from a vector causes enhanced transcription of reporter genes having Sp1/Sp3 sites in their promoters. In similar experiments carried out in mammalian cell lines (see Ding et al., (1999) J. Biol. Chem. 274: 19573-19580; Galvagni et al., (2001) J. Mol. Biol. 306: 985-996; Wilkerson et al., (2002) J Cell Biochem. 86: 716-725) expression of Sp3 and Sp1 had an additive effect on the transcription of such reporter constructs. Ge et al., (2002) Biochem. Biophys. Acta report transiently expressed Sp1 in Drosophila cells results in synergistic effects between Sp1 and Sp3 proteins on expression of reporter genes driven by a promoter that has multiple Sp1/Sp3 sites. In most cases in which Sp3 functions as an activator of transcription, it exhibits weaker activity than Sp1. Significantly, Davis et al., (2003) Nucleic Acids Res. 31: 1097-1107 reported that the role of Sp3 as an activator or repressor of transcription of genes is not completely resolved, as there are reports of both activities.

Both Sp1 and Sp3 protein are known to be modified by glycosylation. Sp1 exists in two forms: a phosphorylated (105 kDa) form and a non-phosphorylated (95 kDa) form (see Rohlff et al., (1997) J. Biol. Chem. 272: 21137-21141; and Armstrong et al., (1997) J. Biol. Chem. 272: 13489-13495). Black et al., (1999) J. Biol. Chem. 274: 1207-1215 report that serum stimulation increases the phosphorylation of Sp1 protein in mouse and human fibroblasts that are in-mid-GI, but does not affect Sp1 protein levels. Other recent publications demonstrate that phosphorylation of the specific sites of Sp1 protein renders the protein more active as a transcription factor for specific genes, while Leggett et al., (1995) J. Biol. Chem. 270: 25879-25884 report that phosphorylation of Sp1 protein results in a 10-fold loss of affinity for its DNA binding sequence.

In one aspect, the present invention provides a nucleic acid which hybridizes under high or low stringency conditions to a nucleic acid which encodes an Sp1 or Sp3 polypeptide having all or a portion of an amino acid sequence shown in one of SEQ ID NO:1 or 3. Appropriate stringency conditions that promote DNA hybridization, for example, 6.0× sodium chloride/sodium citrate (SSC) at about 45°, followed by a wash of 2.0×SSC at 50° C., are known to those skilled in the art or can be found, e.g., in Current Protocols in Molecular Biology, John Wiley & Sons, N.Y. (1989). For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C.

The invention provides a Sp1 and Sp3 nucleic acid subsequences comprising substantially purified oligonucleotide, wherein the oligonucleotide comprises a region of nucleotide sequence which hybridizes under stringent conditions to at least 10 consecutive nucleotides of sense or antisense sequence of SEQ ID NO:1 or 3, or naturally occurring mutants thereof. Nucleic acid probes, which are specific for Sp1 or Sp3 are contemplated by the present invention. In useful embodiments, the Sp1 or Sp3 subsequence comprises at least about 10 consecutive Sp1 (SEQ ID NO:1) or Sp3 (SEQ ID NO:3) nucleotides and been optimized for one or more Sp1/Sp3 inhibitory function (e.g., has been selected for optimal hybridization with Sp1 or Sp3 mRNA, and, e.g., is functionally associated with hammerhead ribozyme sequences). Particular Sp1 or Sp3 oligonucleotide subsequences may be at least 10 nucleotides in length, though Sp1/Sp3 subsequences of 20, 30, 50, 100, or 150 nucleotides in length are also contemplated.

4.3.1. Binding Site (Decoy) Oligonucleotides

Some aspects of the invention make use of materials and methods for effecting repression of one or more target genes (e.g., Sp1 or Sp3) by means of an Sp1/Sp3 binding site oligonucleotide which, e.g., acts as a transcription factor decoy and prevents the binding of Sp1 and/or Sp3 to natural binding sites occurring in Sp1- and/or Sp3-responsive gene promoters. Sp1 and Sp3 have closely related DNA binding specificities corresponding to G-rich elements such as the so-called GC-box (e.g., GGGCGG or GGGGCGGGG) or the related GT/CACCC-box (e.g., GGTGTGGGG). In this aspect of the invention, oligonucleotides which include one or more of these Sp1 and/or Sp3-binding sequences are administered to a subject or administered to an S1/Sp3-expressing cell to prevent or block Sp1 and/or Sp3-mediated gene regulation (see Example 5.5 appearing below).

Synthetic double-stranded oligodeoxynucleotides (ODNs) as ‘decoy’ cis elements block the binding of transcription factors to promoter regions of target genes, resulting in the inhibition of gene transactivation in vitro and in vivo (see, e.g., Bielinska et al., (1990) Science 250: 997-1000). Furthermore, certain studies have described application of decoy strategy as in vivo gene therapy (see, e.g., Morishita et al., (1997) Nature Med. 8: 894-899; and Kawamura et al. (1999) Gene Therapy 6: 91-7). Still further, it has been shown that administration of NF-kB ODNs reduced the severity of chronic inflammatory reactions such as streptococcal cell wall- and collagen-induced arthritis in rats (see, e.g., Tomita et al., (1999) Arthr. Rheum. 42: 2532-42; and see also D'Acquisto et al., (2000) Gene Therapy 7: 1731-37).

Methods of creating suitable Sp1/Sp3 binding site oligonucleotides are known in the art, e.g., as described in detail in U.S. Pat. No. 6,060,310, the contents of which, along with each of the above-cited references, are incorporated by reference herein in their entirety. Briefly, the method utilizes the displacement of transcription factors from their enhancer binding sites offers a means of regulating gene expression. For example, it has been shown that prokaryotic repressors can function as negative regulators of eukaryotic promoters (Hu and Davidson (1988) Cell 53: 927). This observation suggests that displacement of activating proteins might provide a general strategy for gene-specific repression in eukaryotes. In one approach, promoter competition is utilized whereby plasmids containing cis-acting elements (e.g,. an Sp1/Sp3 binding site such as a GC-box), are introduced in high copy number into cells (see, e.g., Wang and Calame (1986) Cell 47: 241). At high copy number, a majority of the transcription factors can be competitively bound away from the natural enhancer sequences with gene expression accordingly regulated. Because these plasmids must be maintained uniformly in large numbers of cells, this approach is useful but not preferred.

Accordingly, the approach of the presently claimed invention further utilizes oligonucleotides, modified to facilitate entry into cells that compete with the native cellular Sp1/Sp3-binding enhancers for binding to transcription factors. Transcription factor decoys of the presently claimed invention are recognized and bound by the Sp1 and/or Sp3 transcription factors such that the factors can no longer bind to native response elements and regulate gene expression. Decoys can comprise one or more duplex nucleic acid structures. These structures are recognized by the DNA binding domain of the target transcription factors (i.e., Sp1 and/or Sp3). The present invention is not intended to be limited to decoys with duplex structures however, as any nucleic acid structure that binds to the DNA binding domains are included. The decoys can comprise the consensus sequence for the targeted transcription factor. A consensus sequence is identified as the sequence that, on average (e.g., in the most genes studied thus far or in binding affinity studies), binds with the highest affinity to its associated transcription factor(s). However, the decoys of the present invention are not limited to sequences comprising the consensus sequence. A variety of enhancers, with sequences slightly divergent from a consensus sequence, are often known to bind to the associated transcription factor. The present invention contemplates decoys comprising sequences from such known enhancers. The present invention further contemplates decoys comprising sequences similar to a consensus sequence and other known enhancers. Any decoy that has affinity for the target transcription factor(s) is suitable for use as a decoy and is contemplated by the presently claimed invention.

The Sp1/Sp3 G/C-box binding site, 5′-GGGGCGGGG-3′, has been described as the consensus sequence for the cis-element that directs Sp1-induced gene transcription. The Sp1/Sp3 decoy oligonucleotides of the present invention comprise sequences that contain one or more such Sp1/Sp3 binding sites. The invention includes various Sp1/Sp3 decoy nucleic acid sequences including duplex, hairpin, and cruciform oligonucleotides containing an Sp1/Sp3-binding consensus sequence (e.g., decoys), which, in certain aspects of the invention, inhibit cancerous cell growth without adversely or minimally affecting non-cancerous cells. Nucleic acid sequences with one or more bases different from an Sp1 or Sp3 consensus sequence may still be recognized by the transcription factors. Thus a range of sequences may be effectively employed as decoys. By selecting sequences sufficiently divergent from the consensus sequence, decoys can be generated with varying affinities (e.g., potencies).

In some embodiments, the oligonucleotides of the present invention are synthesized with modified phosphodiester bonds, including, but not limited to phosphorothioate, phosphoramidite, or methyl phosphonate derivatives. However, the present invention is not limited to the use of oligonucleotides with modified phosphodiester bonds. The modified oligonucleotides can be synthesized in large amounts and are relatively resistant to nucleases (Zon (1988) Pharm. Res. 5:539; and Agrawal et al., (1988) Proc. Natl. Acad. Sci. (USA) 85:7079). Because of their increased cell permeability and stability, such compounds have been used as mRNA antisense agents (Crooke (1992) Ann. Rev. Pharmacol. Toxicol. 32: 329; and Roush (1997) Science 276: 1192). However, unlike the mRNA antisense applications, this aspect of the invention takes advantage of these features to provide a means for directly targeting transcription factors rather than mRNA. Furthermore, the present invention provides novel methods and compositions for globally controlling the expression of genes that are regulated through Sp1 and/or Sp3, unlike the antisense method, which only target mRNA for one specific gene product.

In some embodiments of the present invention, the oligonucleotides are palindromic cis-transcription elements comprising a synthetic single-stranded oligonucleotide composed of the Sp1/Sp3 cis-element that self-hybridized to form a duplex. When introduced into cells, these oligonucleotides act as decoys for the Sp1 and/or Sp3 transcription factors and interfere with the cis-element-directed transcription. While the present invention is not limited to any particular mechanism, it is known that perfect palindromes are capable of forming strong hairpin structures. Such structures may be formed by palindromic decoys, facilitating enhanced binding to the target transcription factors.

A similar approach can be used for a cis-element that is not palindromic. In this case, two synthetic single-stranded oligonucleotides, each composed of the sense- and antisense-cis-element, respectively, in combination can be used as the transcription factor decoy. These single-stranded oligonucleotides can contain multiple copies of the cis-element. In one preferred embodiment, the CRE-palindrome comprises a triplet repeat of the Sp1 consensus sequence: 5′-GGGGCGGGG-3′.

In other embodiments, synthetic oligonucleotides designed to form hairpin structures and comprising a cis-transcription element were used as transcription factor decoys. Recent evidence has indicated that DNA hairpin formation may represent an additional level of transcriptional control. For example, 23-bp synthetic oligonucleotide of human enkephalin gene enhancer has been shown to undergo a reversible conformational change from a duplex to a cruciform structure of two hairpins (McMurray et al., (19911) Proc. Natl. Acad. Sci. (USA) 88:666). Within the enkephalin enhancer, mutations, which stabilize or destabilize a cruciform structure, resulted in increased or decreased transcription, respectively, without affecting the transcription factor binding.

Hairpin oligonucleotides, containing a duplex portion with an Sp1/Sp3 binding site, may be introduced into cells and successfully function as decoys to alter gene expression. In other embodiments, two hairpin forming synthetic oligonucleotides, each containing one of the sense- and antisense-cis-elements, respectively, and complementary to the other, in combination form a cruciform DNA. Such cruciform DNA can increase the potency of the transcription factor decoy to inhibit gene transcription. Similar DNA structures are known to be generated during genetic recombination and from palindromic sequences under the effect of supercoiling, indicating a biological role for such structures.

4.3.2 Triple Helix-Forming Oligonucleotides

Some embodiments of the invention make use of materials and methods for effecting repression of one or more target genes (e.g., Sp1 or Sp3) by means of Triple Helix-Forming Oligonucleotides (TFOs). TFOs recognize and bind specific sequences via the major groove of duplex DNA and allow for gene targeting in vivo. Binding to a gene promoter sequence corresponding to a transcription factor (e.g,. an Sp1 or Sp3) binding site can block the particular gene promoter from activation by the transcription factor and thereby serve as an Sp1/Sp3-inhibitory agent. One advantage of TFO technology is that it can efficiently target for inhibition those chromosomal sites (i.e. Sp1/Sp3 binding sites such as GC-boxes) to be specifically inhibited. Indeed it has been demonstrated that triplex formation at a promoter can block the binding of various transcription factors, including Sp1, thereby inhibiting transcriptional initiation (see, e.g., Chan et al., (1997) J. Mol. Med. 75: 267).

A DNA triple helix can form when a TFO lies in the major groove of intact duplex DNA. The most stable structures assemble on polypurine: polypyrimidine sequences with hydrogen bonds formed between the bases in the third strand and those in the purine strand of the duplex. The purine or pyrimidine motif third strands may be involved in triplex formation depending on the target sequence, and a binding code for the design of the third strands has been suggested (see e.g., Letai et al., (1988) Biochemistry 27: 9108-12). Triplex formation has been observed to occur efficiently at hompurine regions. The TFO binds in the major groove of DNA, forming Hoogsteen or reverse Hoogsteen hydrogen bonds with bases in the purine-rich strand. The TFO itself may consist of either pyrimidies or pures. Pyrimidine-containing TFOs generally bind parallel to the purine-rich strand; sequence specificity is mediated by specific binding of thymine bases to A:T base pairs and protonated cytosine bases (C+) to G:C base pairs. Because cytosine is only protonated under acidic pH conditions, pyrimidine TFOs usually do not efficiently bind to duplex DNA without base modification. Purine-containing oligonucleotides bind antiparallel to the purine-rich strand in the target DNA, and the binding occurs readily at physiological pH. Sequence specificity is mediated by binding of G to G: C and T to A:T base pairs. Accordingly, a useful TFO for targeting Sp1/Sp3 binding site may be rationally designed (see e.g., Carbone et al., (2003) Nucleic Acid Res. 31: 833-843; using an Sp1-targeting Ets2-TFO of sequence 5′-TGGGTGGTTGGTGGTGGGTGGTGGG-3′ (SEQ ID NO:5) targeting the Ets2 Sp1 binding region with G residues opposite G:C base pairs and T residues opposite A:T base pairs).

4.3.3. Sp1/Sp3-Targeted Antisense, Ribozymes and DNA Enzymes

Ribozymes and antisense oligonucleotides that are targeted to Sp1 and Sp3 effect Sp1/Sp3 inhibition by targeting degradation of the corresponding Sp1 or Sp3 mRNAs and/or by inhibiting protein translation from these messenger RNAs. The Sp1 and Sp3 nucleic acids described above provide useful sequences for the design and synthesis of these Sp1 and Sp3 ribozymes and antisense oligonucleotides. Methods of design and synthesis of ribozymes and antisense oligonucleotides are known in the art. Additional guidance is provided herein.

One issue in designing specific and effective mRNA-targeted ribozymes and antisense oligonucleotides (antisense ODNs) is that of identifying accessible sites of antisense pairing within the target mRNA (which is itself folded into a partially self-paired secondary structure). A combination of computer-aided algorithms for predicting RNA pairing accessibility and molecular screening allow for the creation of specific and effective ribozymes and/or antisense oligonucleotides directed against most mRNA targets. Indeed several approaches have been described to determine the accessibility of a target RNA molecule to antisense or ribozyme inhibitors. One approach uses an in vitro screening assay applying as many antisense oligodeoxynucleotides (antisense ODNs) as possible (see Monia et al., (1996) Nature Med. 2:668-675; and Milner et al., (1997) Nature Biotechnol. 15:537-541). Another utilizes random libraries of ODNs (Ho et al., (1996) Nucleic Acids Res. 24:1901-1907; Birikh et al., (1997) RNA 3:429-437; and Lima et al., (1997) J. Biol. Chem. 272:626-638). The accessible sites can be monitored by RNase H cleavage (see Birikh et al, supra; and Ho et al., (1998) Nature Biotechnol. 16:59-63). RNase H catalyzes the hydrolytic cleavage of the phosphodiester backbone of the RNA strand of a DNA-RNA duplex.

In another approach, a pool of semi-random, chimeric chemically synthesized ODNs is used to identify accessible sites cleaved by RNase H on an in vitro synthesized RNA target. Primer extension analyses are then used to identify these sites in the target molecule (see Lima et al., supra). Other approaches for designing antisense targets in RNA are based upon computer assisted folding models for RNA. Several reports have been published on the use of random ribozyme libraries to screen effective cleavage (see Campbell et al., (1995) RNA 1:598-609; Lieber et al., (1995) Mol. Cell Biol. 15: 540-551; and Vaish et al., (1997) Biochemistry 36:6459-6501).

Other in vitro approaches, which utilize random or semi-random libraries of ODNs and RNase H may be more useful than computer simulations (Lima et al., supra). However, use of in vitro synthesized RNA does not predict the accessibility of antisense ODNs in vivo because recent observations suggest that annealing interactions of polynucleotides are influenced by RNA-binding proteins (see Tsuchihashi et al., (1993) Science 267:99-102; Portman et al., (1994) EMBO J. 13:213-221; and Bertrand and Rossi (1994) EMBO J. 13:2904-2912). U.S. Pat. No. 6,562,570, the contents of which are incorporated herein by reference, provides compositions and methods for determining accessible sites within an mRNA in the presence of a cell extract, which mimics in vivo conditions.

Briefly, this method involves incubation of native or in vitro-synthesized RNAs with defined antisense ODNs, ribozymes or DNAzymes, or with a random or semi-random ODN, ribozyme or DNAzyme library, under hybridization conditions in a reaction medium which includes a cell extract containing endogenous RNA-binding proteins, or which mimics a cell extract due to presence of one or more RNA-binding proteins. Any antisense ODN, ribozyme or DNAzyme, which is complementary to an accessible site in the target RNA will hybridize to that site. When defined ODNs or an ODN library is used, RNase H is present during hybridization or is added after hybridization to cleave the RNA where hybridization has occurred. RNase H can be present when ribozymes or DNAzymes are used, but is not required, since the ribozymes and DNAzymes cleave RNA where hybridization has occurred. In some instances, a random or semi-random ODN library in cell extracts containing endogenous mRNA, RNA-binding proteins and RNase H is used.

Next, various methods can be used to identify those sites on target RNA to which antisense ODNs, ribozymes or DNAzymes have bound and cleavage has occurred. For example, terminal deoxynucleotidyl transferase-dependent polymerase chain reaction (TDPCR) may be used for this purpose (see Komura and Riggs (1998) Nucleic Acids Res. 26:1807-11). A reverse transcription step is used to convert the RNA template to DNA, followed by TDPCR. In this invention, the 3′ termini needed for the TDPCR method is created by reverse transcribing the target RNA of interest with any suitable RNA dependent DNA polymerase (e.g., reverse transcriptase). This is achieved by hybridizing a first ODN primer (P1) to the RNA in a region which is downstream (i.e., in the 5′ to 3′ direction on the RNA molecule) from the portion of the target RNA molecule which is under study. The polymerase in the presence of dNTPs copies the RNA into DNA from the 3′ end of P1 and terminates copying at the site of cleavage created by either an antisense ODN/RNase H, a ribozyme or a DNAzyme. The new DNA molecule (referred to as the first strand DNA) serves as first template for the PCR portion of the TDPCR method, which is used to identify the corresponding accessible target sequence present on the RNA.

For example, the TDPCR procedure may then be used, i.e., the reverse-transcribed DNA with guanosine triphosphate (rGTP) is reacted in the presence of terminal deoxynucleotidyl transferase (TdT) to add an (rG)2-4 tail on the 3′ termini of the DNA molecules. Next is ligated a double-stranded ODN linker having a 3′2-4 overhang on one strand that base-pairs with the (rG)2-4 tail. Then two PCR primers are added. The first is a linker primer (LP) that is complementary to the strand of the TDPCR linker which is ligated to the (rG)2-4 tail (sometimes referred to as the lower strand). The other primer (P2) can be the same as P1, but may be nested with respect to P1, i.e., it is complementary to the target RNA in a region which is at least partially upstream (i.e., in the 3′ to 5′ direction on the RNA molecule) from the region which is bound by P1, but it is downstream of the portion of the target RNA molecule which is under study. That is, the portion of the target RNA molecule, which is under study to determine whether it has accessible binding sites is that portion which is upstream of the region that is complementary to P2. Then PCR is carried out in the known manner in presence of a DNA polymerase and dNTPs to amplify DNA segments defined by primers LP and P2. The amplified product can then be captured by any of various known methods and subsequently sequenced on an automated DNA sequencer, providing precise identification of the cleavage site. Once this identity has been determined, defined sequence antisense DNA or ribozymes can be synthesized for use in vitro or in vivo.

Antisense intervention in the expression of specific genes can be achieved by the use of synthetic antisense oligonucleotide sequences (see, e.g., Lefebvre-d'Hellencourt et al (1995) Eur. Cyokine Netw. 6:7; Agrawal (1996) TIBTECH 14: 376; and Lev-Lehman et al., (1997) Antisense Therap. Cohen and Smicek, eds. (Plenum Press, New York)). Briefly, antisense oligonucleotide sequences may be short sequences of DNA, typically 15-30mer but may be as small as 7mer (see Wagner et al., (1994) Nature 372: 333) designed to complement a target mRNA of interest and form an RNA:AS duplex. This duplex formation can prevent processing, splicing, transport or translation of the relevant mRNA. Moreover, certain AS nucleotide sequences can elicit cellular RNase H activity when hybridized with their target mRNA, resulting in mRNA degradation (see Calabretta et al., (1996) Semin. Oncol. 23:78). In that case, RNase H will cleave the RNA component of the duplex and can potentially release the AS to further hybridize with additional molecules of the target RNA. An additional mode of action results from the interaction of AS with genomic DNA to form a triple helix that may be transcriptionally inactive.

Antisense induced loss-of-function phenotypes related with cellular development have been shown for the glial fibrillary acidic protein (GFAP), for the establishment of tectal plate formation in chick and for the N-myc protein, responsible for the maintenance of cellular heterogeneity in neuroectodermal cultures (ephithelial vs. neuroblastic cells, which differ in their colony forming abilities, tumorigenicity and adherence, see Rosolen et al., (1990) Cancer Res. 50: 6316; and Whitesell et al., (1991) Mol. Cell Biol. 11: 1360). Antisense oligonucleotide inhibition of basic fibroblast growth factor (bFgF), having mitogenic and angiogenic properties, suppressed 80% of growth in glioma cells (see Morrison (1991) J. Biol. Chem. 266: 728) in a saturable and specific manner.

In as a non-limiting example of, addition to, or substituted for, an antisense sequence as discussed herein above, ribozymes may be utilized for suppression of gene function. This is particularly necessary in cases where antisense therapy is limited by stoichiometric considerations. Ribozymes can then be used that will target the same sequence. Ribozymes are RNA molecules that possess RNA catalytic ability that cleave a specific site in a target RNA. The number of RNA molecules that are cleaved by a ribozyme is greater than the number predicted by a 1:1 stoichiometry (see Hampel and Tritz (1989) Biochem. 28: 4929-33; and Uhlenbeck (1987) Nature 328: 596-600). Therefore, the present invention also allows for the use of the ribozyme sequences targeted to an accessible domain of an Sp1 or Sp3 mRNA species and containing the appropriate catalytic center. The ribozymes are made and delivered as known in the art and discussed further herein. The ribozymes may be used in combination with the antisense sequences.

Ribozymes catalyze the phosphodiester bond cleavage of RNA. Several ribozyme structural families have been identified including Group I introns, RNase P, the hepatitis delta virus ribozyme, hammerhead ribozymes and the hairpin ribozyme originally derived from the negative strand of the tobacco ringspot virus satellite RNA (sTRSV) (see Sullivan (1994) Investig. Dermatolog. (Suppl.) 103: 95S; and U.S. Pat. No. 5,225,347). The latter two families are derived from viroids and virusoids, in which the ribozyme is believed to separate monomers from oligomers created during rolling circle replication (see Symons (1989) TIBS 14: 445-50; Symons (1992) Ann. Rev. Biochem. 61: 641-71). Hammerhead and hairpin ribozyme motifs are most commonly adapted for trans-cleavage of mRNAs for gene therapy. The ribozyme type utilized in the present invention is selected as is known in the art. Hairpin ribozymes are now in clinical trial and are a particularly useful type. In general the ribozyme is from 30-100 nucleotides in length.

Ribozyme molecules designed to catalytically cleave a target mRNA transcript (e.g., Sp1 (SEQ ID NO:1) or Sp3 (SEQ ID NO:3) can also be used to prevent translation of mRNA (see, e.g., PCT International Pub. WO90/11364; Sarver et al., (1990) Science 247:1222-1225 and U.S. Pat. No. 5,093,246). While ribozymes that cleave mRNA at site specific recognition sequences can be used to destroy particular mRNAs, the use of hammerhead ribozymes is particularly useful. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach ((1988) Nature 334: 585).

The ribozymes of the present invention also include RNA endoribonucleases (hereinafter “Cech-type ribozymes”) such as the one which occurs naturally in Tetrahymena thermophila (known as the IVS, or L-19 IVS RNA), and which has been extensively described by Thomas Cech and collaborators (see Zaug et al., (1984) Science 224:574-578; Zaug and Cech (1986) Science 231:470-475; Zaug, et al., (1986) Nature 324:429-433; International patent application No. WO88/04300; Been and Cech (1986) Cell 47:207-216). The Cech-type ribozymes have an eight base pair active site, which hybridizes to a target RNA sequence where after cleavage of the target RNA takes place. The invention encompasses those Cech-type ribozymes, which target eight base-pair active site sequences. While the invention is not limited to a particular theory of operative mechanism, the use of hammerhead ribozymes in the invention may have an advantage over the use of Sp1/Sp3-directed antisense, as recent reports indicate that hammerhead ribozymes operate by blocking RNA translation and/or specific cleavage of the mRNA target.

As in the antisense approach, the ribozymes can be composed of modified oligonucleotides (e.g., for improved stability, targeting, etc.) and are delivered to cells expressing the target mRNA. A useful method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes, unlike antisense molecules, are catalytic, a lower intracellular concentration is required for efficiency.

Nuclease resistance, where needed, is provided by any method known in the art that does not substantially interfere with biological activity of the antisense oligodeoxynucleotides or ribozymes as needed for the method of use and delivery (Iyer et al., (1990) J. Org. Chem. 55: 4693-99; Eckstein (1985) Ann. Rev. Biochem. 54: 367402; Spitzer and Eckstein (1988) Nucleic Acids Res. 18: 11691-704; Woolf et al., (1990) Nucleic Acids Res. 18: 1763-69; and Shaw et al., (1991) Nucleic Acids Res. 18: 11691-704). Non-limiting representative modifications that can be made to antisense oligonucleotides or ribozymes in order to enhance nuclease resistance include modifying the phosphorous or oxygen heteroatom in the phosphate backbone, short chain alkyl or cycloalkyl intersugar linkages or short chain heteroatomic or heterocyclic intersugar linkages. These include, e.g., preparing 2′-fluoridated, O-methylated, methyl phosphonates, phosphorothioates, phosphorodithioates and morpholino oligomers. For example, the antisense oligonucleotide or ribozyme may have phosphorothioate bonds linking between four to six 3′-terminus nucleotide bases. Alternatively, phosphorothioate bonds may link all the nucleotide bases. Phosphorothioate antisense oligonucleotides do not normally show significant toxicity at concentrations that are effective and exhibit sufficient pharmacodynamic half-lives in animals (see Agarwal et al., (1996) TIBTECH 14: 376) and are nuclease resistant. Alternatively the nuclease resistance for the AS-ODN can be provided by having a 9 nucleotide loop forming sequence at the 3′-terminus having the nucleotide sequence CGCGAAGCG. The use of avidin-biotin conjugation reaction can also be used for improved protection of AS-ODNs against serum nuclease degradation (see Boado and Pardridge (1992) Bioconj. Chem. 3: 519-23). According to this concept the AS-ODN agents are monobiotinylated at their 3′-end. When reacted with avidin, they form tight, nuclease-resistant complexes with 6-fold improved stability over non-conjugated ODNs.

Other studies have shown extension in vivo of AS-oligodeoxynucleotides (Agarwal et al., (1991) Proc. Natl. Acad. Sci. (USA) 88: 7595). This process, presumably useful as a scavenging mechanism to remove alien AS-oligonucleotides from the circulation, depends upon the existence of free 3′-termini in the attached oligonucleotides on which the extension occurs. Therefore partial phosphorothioate, loop protection or biotin-avidin at this important position should be sufficient to ensure stability of these AS-oligodeoxynucleotides.

The present invention also includes use of all analogs of, or modifications to, an oligonucleotide of the invention that does not substantially affect the function of the oligonucleotide or ribozyme. Such substitutions may be selected, for example, in order to increase cellular uptake or for increased nuclease resistance as is known in the art. The term may also refer to oligonucleotides or ribozymes, which contain two or more distinct regions where analogs have been substituted.

The nucleotides can be selected from naturally occurring or synthetically modified bases. Naturally occurring bases include adenine, guanine, cytosine, thymine and uracil. Modified bases of the oligonucleotides include, but are not limited to, xanthine, hypoxanthine, 2-aminoadenine, 6-methyl, 2-propyl and other alkyl adenines, 5-halo uracil, 5-halo cytosine, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogs of nucleotides can be prepared wherein the structure of the nucleotide is fundamentally altered and that are better suited as therapeutic or experimental reagents. An example of a nucleotide analog is a peptide nucleic acid (PNA) wherein the deoxyribose (or ribose) phosphate backbone in DNA (or RNA) is replaced with a polyamide backbone, which is similar to that found in peptides. PNA analogs have been shown to be resistant to degradation by enzymes and to have extended lives in vivo and in vitro. Further, PNAs have been shown to bind stronger to a complementary DNA sequence than a DNA molecule. This observation is attributed to the lack of charge repulsion between the PNA strand and the DNA strand. Other modifications that can be made to oligonucleotides include polymer backbones, morpholino polymer backbones (see, e.g., U.S. Pat. No. 5,034,506, the contents of which are incorporated herein by reference), cyclic backbones, or acyclic backbones, sugar mimetics or any other modification including which can improve the pharmacodynamics properties of the oligonucleotide.

A further aspect of the invention relates to the use of DNA enzymes to decrease expression of the target mRNA, e.g., Sp1 or Sp3. DNA enzymes incorporate some of the mechanistic features of both antisense and ribozyme technologies. DNA enzymes axe designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design DNA enzyme that specifically recognizes and cleaves a target nucleic acid, one of skill in the art must first identify the unique target sequence. This can be done using the same approach as outlined for antisense oligonucleotides. In certain instances, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that targets the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined herein. Additionally, one of skill in the art will recognize that, like antisense oligonucleotides, DNA enzymes can be optionally modified to improve stability and improve resistance to degradation.

The synthetic nuclease resistant antisense oligodeoxynucleotides, ribozymes, etc. of the present invention can be synthesized by any method known in the art. For example, an Applied Biosystems 380B DNA synthesizer can be used. Final purity of the oligonucleotides or ribozymes is determined as is known in the art.

4.3.4 RNA Interference

Some embodiments of the invention make use of materials and methods for effecting repression of one or more target genes (e.g., Sp1 or Sp3) by means of RNA interference (RNAi). RNAi is a process of sequence-specific post-transcriptional gene repression that can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR (double-stranded RNA-activated protein kinase), which in its active form phosphorylates the translation initiation factor eIF2 to shut down all protein synthesis, and 2′, 5′ oligoadenylate synthetase (2′, 5′-AS), which synthesizes a molecule that activates RNase L, a nonspecific enzyme that targets all mRNAs. The nonspecific pathway may represent a host response to stress or viral infection, and, in general, the effects of the nonspecific pathway are minimized in particularly useful methods of the present invention. Significantly, longer dsRNAs appear to be required to induce the nonspecific pathway and, accordingly, dsRNAs shorter than about 30 bases pairs are particular useful to effect gene repression by RNAi (see, e.g., Hunter et al., (1975) J. Biol. Chem. 250: 409-17; Manche et al., (1992) Mol. Cell Biol. 12: 5239-48; Minks et al., (1979) J. Biol. Chem. 254: 10180-3; and Elbashir et al., (2001) Nature 411: 494-8).

RNAi has been shown to be effective in reducing or eliminating the expression of a target gene in a number of different organisms including Caenorhabdiffis elegans (see e.g., Fire et al., (1998) Nature 391: 806-11), mouse eggs and embryos (Wianny et al., (2000) Nature Cell Biol. 2: 70-5; and Svoboda et al., (2000) Development 127: 4147-56), and cultured RAT-1 fibroblasts (Bahramina et al., (1999) Mol. Cell Biol. 19: 274-83), and appears to be an anciently evolved pathway available in eukaryotic plants and animals (Sharp (2001) Genes Dev. 15: 485-90).

RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see, e.g., Bass (2001) Nature 411: 428-9). In mammalian cells, siRNAs are effective at concentrations that are several orders of magnitude below the concentrations typically used in antisense experiments (Elbashir et al., (2001) Nature 411: 494-8).

Certain double stranded oligonucleotides used to effect RNAi are less than 30 base pairs in length and may comprise about 25, 24, 23, 22, 21, 20, 19, 18 or 17 base pairs of ribonucleic acid. Optionally, the dsRNA oligonucleotides of the invention may include 3′ overhang ends. Non-limiting exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al., (2001) Nature 411: 494-8).

Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g., Expedite RNA phophoramidites and thymidine phosphoramidite (Proligo, Germany)). Synthetic oligonucleotides may be deprotected and gel-purified using methods known in the art (see e.g., Elbashir et al., (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence.

The specific sequence utilized in design of the oligonucleotides may be any contiguous sequence of nucleotides contained within the expressed gene message of the target (e.g., of Sp1 (SEQ ID NO:1) or Sp3 (SEQ ID NO:3). Programs and algorithms, known in the art, may be used to select appropriate target sequences. In addition, optimal sequences may be selected, as described additionally above, utilizing programs designed to predict the secondary structure of a specified single stranded nucleic acid sequence and allow selection of those sequences likely to occur in exposed single stranded regions of a folded mRNA. Methods and compositions for designing appropriate oligonucleotides may be found in, for example, U.S. Pat. No. 6,251,588, the contents of which are incorporated herein by reference. mRNA is generally thought of as a linear molecule that contains the information for directing protein synthesis within the sequence of ribonucleotides. However, studies have revealed a number of secondary and tertiary structures exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three-dimensional structure. A number of researchers have measured the binding energies of a large number of RNA duplex structures and have derived a set of rules which can be used to predict the secondary structure of RNA (see e.g., Jaeger et al., (1989) Proc. Natl. Acad. Sci. (USA) 86:7706 (1989); and Turner et al., (1988) Ann. Rev. Biophys. Biophys. Chem. 17:167). The rules are useful in identification of RNA structural elements and, in particular, for identifying single stranded RNA regions, which may represent preferred segments of the mRNA to target for silencing RNAi, ribozyme or antisense technologies. Accordingly, particular segments of the mRNA target can be identified for design of the RNAi mediating dsRNA oligonucleotides as well as for design of appropriate ribozyme and hammerheadribozyme compositions of the invention.

The dsRNA oligonucleotides may be introduced into the cell by transfection with an heterologous target gene using carrier compositions such as liposomes, which are known in the art, e.g., Lipofectamine 2000 (Life Technologies, Rockville Md.) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP encoding pAD3 (Kehlenback et al., (1998) J. Cell. Biol. 141: 863-74). The effectiveness of the RNAi may be assessed by any of a number of assays following introduction of the dsRNAs- These include, but are not limited to, Western blot analysis using antibodies which recognize the targeted gene product following sufficient time for turnover of the endogenous pool after new protein synthesis is repressed, and Northern blot analysis to determine the level of existing target mRNA.

Still further compositions, methods and applications of RNAi technology for use in the invention are provided in U.S. Pat. Nos. 6,278,039, 5,723,750 and 5,244,805, which are incorporated herein by reference.

4.3.5. Sp1/Sp3 Protein Antagonists and Proteolytic Targeting Agents

Dominant Negative

Some embodiments of the invention make use of materials and methods for effecting repression of one or more target genes (e.g., Sp1 or Sp3) by means of Sp1 and/or Sp3-targeted dominant negative proteins. In general, a dominant negative protein is a mutated or otherwise altered derivative of a native protein which is capable of interfering with the function of the native protein. A dominant negative Sp1 or Sp3 refers to a mutated Sp1 or Sp3 gene and/or encoded mutated Sp1 or Sp3 protein which lacks the activity of the wild-type Sp1 or Sp3 protein but which also inhibits activity of the wild-type Sp1 or Sp3 protein when co-expressed along with the wild-type protein.

The design of dominant negative derivatives of transcription factors that interfere with transcription factor function are well known in the art. In brief, dominant negative versions of transcription factors are readily created by, e.g., deleting the transcriptional activation domain. U.S. Pat. No. 5,869,040, for example, describes dominant negative forms of the nuclear proto-oncogene E2F transcription factor protein and methods of their use to treat cancer. The “design rules” for developing particular dominant negative molecules may vary with the structure of the native target (although, in general, transcription factors deleted for their transcriptional-regulatory sequences (e.g., trans-activation domains) but retaining their sequence-specific DNA binding capabilities will function as dominant negative molecules. E2F proto-oncogene encoding a dominant interfering mutant may include alterations in the DNA binding domain and/or the RB binding domains. For example, mutant proto-oncogene polynucleotides derived from wild-type E2F4 (Hijmans et al., (1995) Mol. Cell. Biol. 15: 3082-3089) lack those polynucleotides encoding amino acids 10-83 (DNA binding domain of wild-type E2F4 protein) and/or those polynucleotides encoding amino acids 382 to about 412 (RB binding domain of E2F4 protein). Similarly, Sp1 sequences from four regions (designated A, B, C and D), which contribute to the transcriptional properties of Sp1 (see Courey and Tjian (1988) Cell 55887-98) may be deleted, while retaining the three DNA-binding zinc finger structures, to create suitable Sp1 dominant negative polypeptides of the invention.

A dominant negative form of Sp1 has been created (see Petersohn and Thiel (1996) Eur. J. Biochem. 239: 827-34 and Kavurma and Khachigian (2003) J. Biol. Chem. [June 9; Epub ahead of print]). This dominant-negative (DN) form of Sp1 bears the DNA binding domain, but not the transcriptional activation domain (i.e., amino acids 592-758). Furthermore a vector for overexpressing this DN Sp1, pEBGSp1, has been constructed. Accordingly, expression of of a polypeptide fragment of Sp1 lacking the transcriptional activation domain but carrying the zinc-finger DNA binding domain, e.g,. Sp1 amino acid residues 1-591+759-785, may be used to inhibit Sp1/Sp3 activity since the two factors bind to similar sequences. Furthermore, other DN Sp1 species and analogous forms of Sp3 may be created by deleting or otherwise mutating various portions of each protein and assaying each deleted/mutated form for its ability to activate transcription using an Sp1/Sp3-responsive reporter gene (e.g., one containing upstream synthetic GC boxes) and its ability to bind DNA (e.g., by assaying for binding to a GC box-oligonucleotide in an electrophoretic mobility shift assay (EMSA)). Those deleted, or otherwise mutated, forms of Sp1 or Sp3 which fail to alter transcription of the reporter, but retain the ability to bind DNA are suitable DN Sp1 or DN Sp3 forms of the Sp1/Sp3-inhibitory agents of the invention.

Aptamers

Some embodiments of the invention make use of materials and methods for effecting repression of one or more target genes (e.g., Sp1 or Sp3) by means of Sp1 and/or Sp3-targeted aptamers. Aptamers are nucleic acid ligands which have been selected for their ability to bind to and, in certain instances, inhibit a particular protein or polypeptide target.

Such target-specific aptamers, e.g., Sp1 and/or Sp3-targeted aptamers, may be created using a type of in vitro natural selection for randomly-generated nucleic acid sequences which bind to the selected target, e.g., Sp1 and/or Sp3. This method has been termed “SELEX” (for Systematic Evolution of Ligands by Exponential Enrichment). The SELEX method (hereinafter termed SELEX), and related application is described in, e.g., U.S. Pat. Nos. 5,475,096, 6,083,696, 6,441,158 and 6,458,559, the contents of which are incorporated herein in their entireties. The SELEX process provides a class of products which are referred to as nucleic acid ligands, such ligands having a unique sequence, and which have the property of binding specifically to a desired target compound or molecule. Each SELEX-identified nucleic acid ligand is a specific ligand of a given target compound or molecule. SELEX is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size can serve as targets.

Briefly, the SELEX method involves selection from a mixture of candidates and step-wise iterations of binding, partitioning, and amplification, using the same general selection theme, to achieve virtually any desired criterion of binding affinity and selectivity. Starting from a mixture of nucleic acids, preferably comprising a segment of randomized sequence, the method includes steps of contacting the mixture with the target under conditions favorable for binding, partitioning unbound nucleic acids from those nucleic acids which have bound to target molecules, dissociating the nucleic acid-target pairs, amplifying the nucleic acids dissociated from the nucleic acid-target pairs to yield a ligand-enriched mixture of nucleic acids, then reiterating the steps of binding, partitioning, dissociating and amplifying through as many cycles as desired. A variety of techniques can be used to partition members in the pool of nucleic acids that have a higher affinity to the target than the bulk of the nucleic acids in the mixture.

While not bound by theory, SELEX is based on the observation that within a nucleic acid mixture containing a large number of possible sequences and structures there is a wide range of binding affinities for a given target. A nucleic acid mixture comprising, for example, a 20 nucleotide randomized segment, can have 420 candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection, partition and amplification are repeated until a desired goal is achieved. In the most general case, selection/partition/amplification is continued until no significant improvement in binding strength is achieved on repetition of the cycle. The method may be used to sample as many as about 10¹⁸ different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain subportions of conserved sequence incorporated with randomized sequence. Sequence variation in test nucleic acids can be introduced or increased by mutagenesis before or during the selection/partition/amplification iterations.

For target molecules which are nucleic acid binding proteins, such as Sp1 and Sp3, evolved SELEX ligands may be homologous to the natural ligand since the nucleic acid binding protein target has evolved naturally to present side-chain and/or main-chain atoms with the correct geometry to interact with nucleic acids. Non-nucleic acid binding proteins which have evolved to bind poly-anions such as sulfated glycans (e.g., heparin), or to bind phospholipids or phosphosugars, also have sites into which nucleic acids can fit and make contacts analogous with the natural ligands and/or substrates.

Optionally, where the domain targeted for aptamer-inhibiton does not naturally bind a polyanion ligand, it can be more difficult (but still likely with relatively more rounds of SELEX) to identify oligonucleotides that fit into the substrate or ligand site. For instance, the binding pocket of trypsin contains a carboxyl group, which interacts during catalysis with a lysine or arginine residue on the substrate. An oligonucleotide may not fit into this specific catalytic site because it would not contain a positively charged counter ion. Basic SELEX evolution of oligonucleotide ligands to such a target molecule may result in ligands to a site(s) distant from the substrate site, since the probability of recovering ligands to the substrate site may be low. Similarly, the trans-activation domains of Sp1 and/or Sp3 can be targeted by rational design considerations applying SELEX.

The basic SELEX method may be modified to achieve specific objectives. For example, U.S. Pat. No. 5,707,796, describes the use of SELEX in conjunction with gel electrophoresis to select nucleic acid molecules with specific structural characteristics, such as bent DNA. U.S. Pat. No. 5,763,177, describes a SELEX based method for selecting nucleic acid ligands containing photoreactive groups capable of binding and/or photocrosslinking to and/or photoinactivating a target molecule. U.S. Pat. No. 5,580,737, describes a method for identifying highly specific nucleic acid ligands able to discriminate between closely related molecules, termed “counter-SELEX”. U.S. Pat. No. 5,567,588, describes a SELEX-based method which achieves highly efficient partitioning between oligonucleotides having high and low affinity for a target molecule.

The SELEX method encompasses the identification of high-affinity nucleic acid ligands containing modified nucleotides conferring improved characteristics on the ligand, such as improved in vivo stability or delivery. Examples of such modifications include chemical substitutions at the ribose and/or phosphate and/or base positions. Specific SELEX-identified nucleic acid ligands containing modified nucleotides are described in, e.g., U.S. Pat. No. 5,660,985, which describes oligonucleotides containing nucleotide derivatives chemically modified at the 5- and 2′-positions of pyrimidines, as well as specific RNA ligands to thrombin containing 2′-amino modifications. Also included are highly specific nucleic acid ligands containing one or more nucleotides modified with 2′-amino (2′-NH₂), 2′-fluoro (2′-F), and/or 2′-O-methyl (2′-OMe).

The Sp1 and/or Sp3 aptamer inhibitors of the invention are selected for using the SELEX method described above and further screened for Sp1 and/or Sp3-inhibitory activity using the Sp1/Sp3 activity assays described throughout this application, e.g., inhibition of GC-box DNA-binding activity in an EMSA and/or trans-activation in a GC-box reporter-based cultured cell transfection assay.

Proteolytic Targeting Agents

Yet another means of the invention for inhibiting Sp1 and/or Sp3 directed at the polypeptide level utilizes a method of directed proteolytic targeting to destroy Sp1 and/or Sp3 by the specific recruitment of these proteins to ubiquitin protein ligases. Exemplary methods for obtaining targeted proteolysis by recruitment to ubiquitin protein ligases are described in, e.g., WO 00/22110, the contents of which are incorporated herein in their entirety.

In brief, this method involves targeted proteolysis through recruitment of the targeted polypeptide to a protein ubiquitin ligase. The protein ubiquitin ligases which can be used include E3-type ubiquitin ligases such as the SCF ubiquitin ligases, the HECT ubiquitin ligases or the UBRI ubiquitin ligases. The targeted polypeptide may be a natural target of ubiquitination by these ubiquitin ligases or may be a polypeptide, which is not normally targeted for degradation by ubiquitin conjugation in general or by an E3-type ubiquitin protein ligase of the invention in particular. In general, the target polypeptide is recruited to an E3 ubiquitin ligase complex either by covalent joining of the target polypeptide to a component of the complex (cis targeting) or by noncovalent association of the target polypeptide with a component of the complex (trans targeting). The invention thereby provides for the controlled degradation of any cellular protein, such and Sp1 and/or Sp3 for which the encoding gene has been cloned or for which an interacting polypeptide is known or can easily be elucidated by one of skill in the art.

In one aspect of the invention, the invention provides for the cis targeting of a polypeptide, e.g., by joining the targeted protein (Sp1 or Sp3) to a component of an SCF (Skpl/Cull 1/17-box protein) which serves as an SCF recruitment domain. Suitable SCF recruitment domains include F-box proteins and the targeted polypeptide is produced as an F-box-Sp1 or F-box-Sp3 fusion protein. The F-box polypeptide sequence may be obtained from an F-box-containing protein such as Cdc4p, Grrlp, Met3Op, HOS (human homolog of Slimb), beta TrCP (Slimb), or FWDI (mouse beta TrCP) (see the National Center for Biotechnology Information (NCBI) for sequences and related useful references). Alternatively, an F-box containing polypeptide from a related protein may be used, or a synthetic F-box polypeptide sequence, related in structure to Cdc4p, Pop 1, Pop 2, Grrlp, Met3Op, HOS, beta TrCP, Pop 1, Pop 2 or FVVD I may be used. In certain instances, the F-box polypeptide-Sp1/Sp3 polypeptide fusion protein includes a VYTD40 polypeptide region such as provided by the WD repeats of Cdc4p or as can be obtained from a large family of proteins which contain WD repeat sequences (see e.g., van der Voorn and Ploegh (1992) FEBS Lett 307: 131-4).

The above example demonstrates that a modified Sp1 and/or Sp3 susceptible to proteolysis may be generated for use in certain select aspects of the invention. In general, however the most useful embodiments of this aspect of the invention involve the targeting of a native Sp1 and/or Sp3 polypeptide in trans by recruitment to an SCF E3 ubiquitin ligase complex through trans association with at least one component of the complex. Preferably, such trans targeting of a polypeptide or polypeptides is effected by fusing a “target polypeptide interaction domain” to an F-box polypeptide sequence. The “target polypeptide interaction domains” may be readily obtained by screening a library of naturally-occurring polypeptide sequence such as by using the γeast two-hybrid methodology (Fields and Song (1989) Nature 340:245-6; Gyunis et al., (1993) Cell 75:791-803). Alternatively, synthetic interaction domains may be obtained by any of various peptide display selection methods, such as phage display, which are known in the art.

Sp1 is known to bind a number of polypeptides and other Sp1 and/or Sp3-binding polypeptides may be screened and/or selected for using methods known in the art. For example, Sp1 has been shown to bind to mutant forms of huntingtin, the polyglutamine protein affected in Huntington's disease (see Li et al. (2002) Mol. Cell. Biol. 22: 1277-87) as well as to SV40 capsid polypeptides VP1 and VP2/3 (see Gordon-Shaag et al. (2002) J. Virol. 76: 5915-24). Furthermore, Sp1 and Sp3 has been shown to interact with jun (see Chamboredon et al., (2003) Oncogene 22: 4047-61). The expression in a target host cell (e.g., cancerous or tumor cell) of a “chimeric” or “hybrid” polypeptide containing: (i) a domain of a substrate binding component of an E3 ubiquitin-protein ligase complex, e.g., yeast cdc4p or the human protein hPTrCP, which domain provides a ubiquitination activity, e.g., by recruiting target proteins into an SCF ubiquitin protein ligase complex; and (ii) a domain capable of interacting with an Sp1 and/or Sp target polypeptide, results in the degradation of the Sp1 target polypeptide in the host cell.

4.4 Expression Vectors for Sp1/Sp³ Inhibitory Agents

As described in detail below, the invention provides vectors for the expression of the Sp1 and Sp3-inhibitory agents of the invention. For example, the human U1snRNA promoter-containing vector, described in detail as used to drive expression of the Sp1 and Sp3 ribozymes in the examples which follow, provides one particularly useful expression vector of the invention. Similarly, Sp1 promoter and Sp3 promoter-driven constructs, particularly those providing negative feedback autoregulatory characteristics as described further herein, are particularly useful embodiments of this aspect of the invention.

In addition to viral transfer methods described below, non-viral methods can also be employed to cause expression of Sp1/Sp3 inhibitory agents in the tissue of an animal. Most nonviral methods of gene transfer rely on normal mechanisms used by mammalian cells for the uptake and intracellular transport of macromolecules. In some embodiments, non-viral gene delivery systems of the present invention rely on endocytic pathways for the uptake of the subject Sp1/Sp3 inhibitory agents by the targeted cell. Exemplary gene delivery systems of this type include liposomal derived systems, poly-lysine conjugates, and artificial viral envelopes.

Useful mammalian expression vectors may contain both prokaryotic sequences to facilitate the propagation of the vector in bacteria, and one or more eukaryotic transcription units that are expressed in eukaryotic cells. For example, pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) may be used for transient expression of proteins in eukaryotic cells. Examples of other viral (including retroviral) expression systems are described further below in the description of Sp1/Sp3-inhibitory agent delivery systems. Various methods employed in the preparation of the plasmids and transformation of host organisms are well known in the art. For other suitable expression systems for both prokaryotic and eukaryotic cells, as well as general recombinant procedures, see, e.g., Molecular Cloning A Laboratory Manual, 2nd Ed., ed. by Sambrook, Fritsch and Maniatis (Cold Spring Harbor Laboratory Press: 1989) Chapters 16 and 17. In some instances, it is desirable to express the recombinant Sp1/Sp3 inhibitory agents by the use of a baculovirus expression system. Examples of such baculovirus expression systems include, but are not limited to, pVL-derived vectors (such as pVL1392, pVL1393 and pVL941), pAcUW-derived vectors (such as pAcUWI), and pBlueBac-derived vectors (such as the beta-gal containing pBlueBac III).

In accordance with the subject method, expression constructs of the subject Sp1/Sp3 inhibitory agents may be administered in any biologically effective carrier, e.g., any formulation or composition capable of effectively transfecting cells in vivo with a recombinant Sp1/Sp3 inhibitory agents. Approaches include insertion of the subject gene in viral vectors including, but not limited to, recombinant retroviruses, adenovirus, adeno-associated virus, and herpes simplex virus-1, or recombinant bacterial or eukaryotic plasmids. Viral vectors can be used to transfect cells directly; plasmid DNA can be delivered with the help of, for example, cationic liposomes (lipofectin) or derivatized (e.g., antibody conjugated), poly-lysine conjugates, gramacidin S, artificial viral envelopes or other such intracellular carriers, as well as direct injection of the gene construct or CaPO₄ precipitation carried out in vivo.

In a non-limiting representative embodiment, a gene encoding one of the subject Sp1/Sp3 inhibitory agents is entrapped in liposomes bearing positive charges on their surface (e.g., lipofectins) and (optionally) which are tagged with antibodies against cell surface antigens of the target tissue (Mizuno et al., (1992) No. Shinkei Geka 20:547-551; PCT Pub. WO91/06309; Japanese Patent Appln. 1,047,381; and European Patent Pub. EP-A-43075). For example, lipofection of neuroglioma cells can be carried out using liposomes tagged with monoclonal antibodies against glioma-associated antigen (Mizuno et al., (1992) Neurol. Med. Chir. 32:873-876).

In another embodiment, the Sp1/Sp3-inhibitory agent delivery system comprises an antibody or cell surface ligand, which is cross-linked with a gene binding agent such as poly-lysine (see, for example, PCT Pubs. WO93/04701, WO92/22635, WO92/20316, WO92/19749, and WO92/06180). For example, the subject Sp1/Sp3 inhibitory agents construct can be used to transfect hepatocytic cells in vivo using a soluble polynucleotide carrier comprising an asialoglycoprotein conjugated to a polycation, e.g., poly-lysine (see U.S. Pat. No. 5,166,320). It will also be appreciated that effective delivery of the subject nucleic acid constructs via—mediated endocytosis can be improved using agents, which enhance escape of the gene from the endosomal structures. For instance, whole adenovirus or fusogenic peptides of the influenza HA gene product can be used as part of the delivery system to induce efficient disruption of DNA-containing endosomes (Mulligan et al., (1993) Science 260-926; Wagner et al., (1992) Proc. Natl. Acad. Sci. (USA) 89:7934; and Christiano et al., (1993) Proc. Natl. Acad. Sci. (USA) 90:2122).

A useful approach for in vivo introduction of nucleic acid encoding one of the subject proteins into a cell is by use of a viral vector containing nucleic acid, e.g., a cDNA, encoding the gene product. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells, which have taken up viral vector nucleic acid.

In a particularly useful aspect, the invention provides retrovirus vectors and adeno-associated virus vectors. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see, e.g., Miller (1990) Blood 76:271). Accordingly, recombinant retroviruses can be constructed in which part of the retroviral coding sequence (gag, pol, env) has been replaced by nucleic acid encoding one of the subject Sp1/Sp3 inhibitory agents, rendering the retrovirus replication defective. The replication defective retrovirus is then packaged into virions, which can be used to infect a target cell through the use of a helper virus by standard techniques. Protocols for producing recombinant retroviruses and for infecting cells in vitro or in vivo with such viruses can be found in, e.g., Current Protocols in Molecular Biology (Ausubel, F. M. et al., (eds.) Greene Publishing Associates, (1989), Sections 9.10-9.14) as well as other standard laboratory manuals. Examples of suitable retroviruses include pLJ, pZIP, pWE and pEM, which are well known to those skilled in the art.

Non-limiting examples of suitable packaging virus lines for preparing both ecotropic and amphotropic retroviral systems include ψ Crip, ψ Cre, ψ 2 and ψ Am. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis, et al., (1985) Science 230:1395-1398; Danos and Mulligan (1988) Proc. Natl. Acad. Sci. (USA) 85:6460-6464; Wilson et al., (1988) Proc. Natl. Acad. Sci. (USA) 85:3014-3018; Armentano et al., (1990) Proc. Natl. Acad. Sci. (USA) 87:6141-6145; Huber et al., (1991) Proc. Natl. Acad. Sci. (USA) 88:8039-8043; Ferry et al., (1991) Proc. Natl. Acad. Sci. (USA) 88:8377-8381; Chowdhury et al., (1991) Science 254:1802-1805; van Beusechem et al., (1992) Proc. Natl. Acad. Sci. (USA) 89:7640-7644; Kay et al., (1992) Human Gene Therapy 3:641-647; Dai et al., (1992) Proc. Natl. Acad. Sci. (USA) 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

In choosing retroviral vectors as a gene delivery system for the subject Sp1/Sp3 inhibitory agents, a prerequisite for the successful infection of target cells by most retroviruses, and therefore of stable introduction of the recombinant Sp1/Sp3 inhibitory agents, is that the target cells should be dividing. In general, this requirement will not be a hindrance to use of retroviral vectors to deliver Sp1/Sp3 inhibitory agents constructs. In fact, such limitation on infection can be beneficial in circumstances wherein the tissue (e.g., nontransformed cells) surrounding the target cells does not undergo extensive cell division and is therefore refractory to infection with retroviral vectors.

Furthermore, it has been shown that it is possible to limit the infection spectrum of retroviruses and consequently of retroviral-based vectors, by modifying the viral packaging proteins on the surface of the viral particle (see, for example PCT Pubs. WO93/25234, WO94/06920, and WO94/11524). For instance, strategies for the modification of the infection spectrum of retroviral vectors include: coupling antibodies specific for cell surface antigens to the viral env protein (Roux et al., (1989) Proc. Natl. Acad. Sci. (USA) 86:9079-9083; Julan et al., (1992) J. Gen. Virol. 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g., lactose to convert the env protein to an asialoglycoprotein), as well as by generating fusion proteins (e.g., single-chain antibody/env fusion proteins). This technique, while useful to lirnit or otherwise direct the infection to certain tissue types, and can also be used to convert an ecotropic vector in to an amphotropic vector.

Moreover, use of retroviral gene delivery can be further enhanced by the use of tissue- or cell-specific transcriptional regulatory sequences, which control expression of the Sp1/Sp3 inhibitory agents of the retroviral vector. As mentioned above, particularly useful promoters contain GC boxes that bind to, and are responsive to, the Sp1 and/or Sp3 transcription factors themselves.

Another viral gene delivery system useful in the present invention utilizes adenovirus-derived vectors. The genome of an adenovirus can be manipulated such that it encodes a gene product of interest, but is inactivate in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 dl324 or other strains of adenovirus (e.g., Ad2, Ad3, Ad7, etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) Proc. Natl. Acad. Sci. (USA) 89:6482-6486); hepatocytes (Herz and Gerard (1993) Proc. Natl. Acad. Sci. (USA) 90:2812-2816) and muscle cells (Quantin et al., (1992) Proc. Natl. Acad. Sci. (USA) 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8kb) relative to other gene delivery vectors (see Berkner et al., supra; Haj-Ahmand and Graham (1986) J. Virol. 57:267). Most replication-defective adenoviral vectors in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7: pp. 109-127). Expression of the inserted Sp1/Sp3 inhibitory agents can be under control of, for example, the E1A promoter, the major late promoter (MLP) and associated leader sequences, the E3 promoter, or exogenously added promoter sequences.

Yet another viral vector system useful for delivery of the subject Sp1/Sp3 inhibitory agents is the adeno-associated virus (AAV). As described further in the examples, which follow, this type of expression vector is a particularly useful embodiment of the invention. Adeno-associated virus is a naturally occurring defective virus that requires another virus, such as an adenovirus or a herpes virus, as a helper virus for efficient replication and a productive life cycle. (For a review see Muzyczka et al., (1992), Current Topics in Microbiology and Immunology. 158: 97-129). It is also one of the few viruses that may integrate its DNA into non-dividing cells, and exhibits a high frequency of stable integration (see, for example, Flotte et al., (1992) Am. J. Respir. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) Proc. Natl. Acad. Sci. (USA) 81:6466-6470: Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

Other viral vector systems that may have application in gene therapy have been derived from herpes virus, vaccinia virus, and several RNA viruses. In particular, herpes virus vectors may provide a unique strategy for persistence of the recombinant Sp1/Sp3 inhibitory agents in cells of the central nervous system and occular tissue (Pepose et al., (1994) Invest. Ophthalmol. Vis. Sci. 35:2662-2666).

4.5 Pharmaceutical Formulations and Methods of Treatment

The Sp1/Sp3-inhibitory agent delivery systems of the invention can be introduced into a patient by any of a number of methods, each of which is familiar in the art. For instance, a pharmaceutical preparation of the gene delivery system can be introduced systemically, e.g., by intravenous injection, and specific transduction of the in the target cells occurs predominantly from specificity of transfection provided by the Sp1/Sp3-inhibitory agent delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al., (1994) Proc. Natl. Acad. Sci. (USA) 91:3054-3057).

Moreover, the pharmaceutical preparation can consist essentially of the Sp1/Sp3-inhibitory agent delivery system in an acceptable diluent, or can comprise a slow release matrix in which the gene delivery vehicle is imbedded. Alternatively, where the complete gene delivery system can be produced in tact from recombinant cells, e.g., retroviral packages, the pharmaceutical preparation can comprise one or more cells which produce the gene delivery system. In the case of the latter, methods of introducing the viral packaging cells may be provided by, for example, rechargeable or biodegradable devices. Various slow release polymeric devices have been developed and tested in vivo in recent years for the controlled delivery of drugs, including proteinacious biopharmaceuticals, and can be adapted for release of viral particles through the manipulation of the polymer composition and form. A variety of biocompatible polymers (including hydrogels), including both biodegradable and non-degradable polymers, can be used to form an implant for the sustained release of the viral particles by cells implanted at a particular target site. Such embodiments of the present invention can be used for the delivery of an exogenously purified virus, which has been incorporated in the polymeric device, or for the delivery of viral particles produced by a cell encapsulated in the polymeric device.

By choice of monomer composition or polymerization technique, the amount of water, porosity and consequent permeability characteristics can be controlled. The selection of the shape, size, polymer, and method for implantation can be determined on an individual basis according to the disorder to be treated and the individual patient response. The generation of such implants is generally known in the art. See, for example, Concise Encyclopedia of Medical & Dental Materials, ed. by David Williams (MIT Press: Cambridge, Mass., 1990); and the Sabel et al., U.S. Pat. No. 4,883,666. In another embodiment of an implant, a source of cells producing a recombinant virus is encapsulated in implantable hollow fibers. Such fibers can be pre-spun and subsequently loaded with the viral source (Aebischer et al., U.S. Pat. No. 4,892,538; Aebischer et al., U.S. Pat. No. 5,106,627; Hoffman et al., (1990) Expt. Neurobiol. 110:39-44; Jaeger et al., (1990) Prog. Brain Res. 82:41-46; and Aebischer et al., (1991) J. Biomech. Eng. 113:178-183), or can be co-extruded with a polymer which acts to form a polymeric coat about the viral packaging cells (Lim U.S. Pat. No. 4,391,909; Sefton U.S. Pat. No. 4,353,888; Sugamori et al., (1989) Trans. Am. Artif. Intern. Organs 35:791-799; Sefton et al., (1987) Biotechnol. Bioeng. 29:1135-1143; and Aebischer et al., (1991) Biomaterials 12:50-55). Again, manipulation of the polymer can be carried out to provide for optimal release of viral particles.

To further illustrate the use of the subject method, the therapeutic application of a Sp1/Sp3 inhibitory agents, e.g., by gene therapy, can be used in the treatment of a pancreatic cancer as described further in the examples which follow.

Gene therapy can be used to target glioma cells for expression of recombinant proteins (see, e.g., Miyao et al., (1993) J. Neurosci. Res. 36:472-479; Chen et al., (1994), Proc. Natl. Acad. Sci. (USA), 91:3054-3057; and Takamiya et al., (1993) J. Neurosurg. 79:104-110). Thus, a gene construct for expressing a Sp1/Sp3 inhibitory agents can be delivered to the tumor, e.g., by sterotactic-dependent means. In some embodiments, the gene delivery system is a retroviral vector. Since rapidly growing normal cells are rare in the adult CNS, glioma cells can be specifically transduced with a recombinant retrovirus. For example, the retroviral particle can be delivered into the tumor cavity through an Ommaya tube after surgery, or alternatively, packaging fibroblasts encapsulated in retrievable immunoisolatory vehicles can be introduced into the tumor cavity. In order to increase the effectiveness and decrease the side effects of the retrovirus-mediated gene therapy, tumor-specific promoters can be used to regulate expression of the Sp1/Sp3 inhibitory agents. For example, the promoter regions of glial fibrillary acidic protein (GFAP) and myelin basis protein (MBP) can operably linked to the recombinant gene in order to direct glial cell-specific expression of the recombinant gene construct.

In another embodiment, the subject Sp1/Sp3 inhibitory agents is delivered to a sarcoma, e.g., an osteosarcoma or Kaposi's sarcoma. In a representative embodiment, the gene is provided in a viral vector and delivered by way of a viral particle, which has been derivatized with antibodies immunoselective for an osteosarcoma cell (see, for example, U.S. Pat. Nos. 4,564,517 and 4,444,744; and Singh et al., (1976) Cancer Res. 36:4130-4136).

While it is possible for an Sp1/Sp3-inhibitory agent of the present invention to be administered alone, it is visually administered as a pharmaceutical formulation (composition). The therapeutic Sp1/Sp3-inhibitory agents according to the invention may be formulated for administration in any convenient way for use in human or veterinary medicine, by analogy with other therapeutics (see, e.g., particularly antibody therapeutics such as described in U.S. Pat. Nos. 5,695,757, 6043,347, 6,312,694, 6,368,597, 6,406,693, 6,498,148, 6,440,418, 6,531,128, 6,534,059, 6,537,988, and 6,558,668).

Thus, another aspect of the present invention provides pharmaceutically acceptable compositions comprising a therapeutically-effective amount of one or more of the Sp1/Sp3-inhibitory agents according to the invention may be formulated for administration in any convenient way compositions described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject compounds may be simply dissolved or suspended in sterile water.

The phrase “therapeutically-effective amount,” as used herein, means that amount of a compound, material, or composition comprising a Sp1/Sp3-inhibitory agent according to the invention may be formulated for administration in any convenient way of the present invention which is effective for producing some desired therapeutic effect by inhibiting an Sp1 and/or Sp3 intracellular activity when administered to an animal, at a reasonable benefit/risk ratio applicable to any medical treatment.

The amount of a composition of the invention, which is effective in the treatment or prevention of cancerous or cancerous condition can be determined by standard clinical techniques. In addition, in vitro assays may optionally be employed to help identify optimal dosage ranges. The precise dose to be employed in the formulation will also depend on the route of administration, and the seriousness of the disease or disorder, and is decided according to the judgment of the practitioner and each patient's circumstances. Effective doses may be extrapolated from dose-response curves derived from in vitro or animal model test systems.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject Sp1/Sp3-inhibitory agents from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include, but are not limited to: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As set out above, certain embodiments of the present Sp1/Sp3-inhibitory agents may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al., (1977), J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as, but not limited to, hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The termr “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as, but not limited to, the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Non-limiting representative organic amines useful for the formation of base addition salts include ethylamine, diethylamine, ethylenediamine, ethanolamine, diethanolamine, piperazine and the like. (See, for example, Berge et al., supra) Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form, will generally be that amount of the compound, which produces a therapeutic effect. Generally, out of one hundred per cent, this amount will range from about one per cent to about ninety-nine percent of active ingredient, particularly from about 5 per cent to about 70 per cent, most particularly from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polypropylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made, by molding, in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or in some examples, in a certain portion of the gastrointestinal tract, optionally, in a delayed manner. Examples of embedding compositions, which can be used, include polymeric substances and waxes. The active ingredient can also be in micro encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxypropyl-.beta.-cyclodextrin, may be used to solubilize compounds.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active Sp1/Sp3-inhibitory agents.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include, but are not limited to, powders, sprays, ointments, pastes, creams, lotions, gels, solutions, patches and inhalants. The active compound may be mixed under sterile conditions with a pharmaceutically acceptable carrier, and with any preservatives, buffers, or propellants, which may be required.

The ointments, pastes, creams and gels may contain, in addition to an active compound of this invention, excipients, such as, but not limited to, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as, but not limited to, lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the Sp1/Sp3-inhibitory agents in the proper medium. Absorption enhancers can also be used to increase the flux of the Sp1/Sp3-inhibitory agents across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polypropylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly(anhydrides). Depot injectable formulations are also prepared by entrapping the drug in liposomes or microemulsions, which are compatible with body tissue.

When the compounds of the present invention are administered as pharmaceuticals to humans and animals, they can be given per se or as a pharmaceutical composition containing, for example, 0.1% to 99.5% (or, 0.5 to 90%) of active ingredient in combination with a pharmaceutically acceptable carrier.

The addition of the active compound of the invention to animal feed is maybe accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W. H. Freedman and CO., San Francisco, U.S.A., 1969 or “Livestock Feeds and Feeding” O and B books, Corvallis, Oreg., U.S.A., 1977).

The compounds covered in this invention may be administered alone or in combination with other Sp1/Sp3-inhibitory agents or in combination with a pharmaceutically acceptable carrier of dilutent. The compounds of the invention may be administered intravenously, intramuscularly, intraperitoneally, subcutaneously, topically, orally, or by other acceptable means. The compounds may be used to treat arthritic conditions in mammals (i.e., humans, livestock, and domestic animals), birds, lizards, and any other organism, which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

4.6. Cancers

The invention provides exemplary therapeutics and therapeutic methods for the treatment of cancers and cancerous conditions associated with elevated expression of Sp1 and/or Sp3. In particular, as further supported in the examples that follow, the invention includes compositions and methods of treating pancreatic cancer and fibrosarcomas. Other form of cancer associated with elevated levels of Sp1 and/or Sp3 include, but are not limited to breast cancer, gastric cancer, thyroid cancer and leukemia.

For example, over-expression of Sp1 was found in other types of cancers and/or cancer cell lines. In one study, fourteen biopsy specimens from patients with breast carcinoma and five from patients with benign breast lesions were examined for Sp1 transactivation activity. The authors found that Sp1 binding activity was elevated in all the specimens from the patients with breast carcinoma, and the Sp1 binding activity was undetectable or barely detectable in the benign breast lesions (see Zannetti et al. (2000) Cancer Research 60: 1546-1551). In another study, Kitadai et al. ((1992) Biochem. Biophys. Research Comm. 189:1342-1348) examined the Sp1 mRNA levels in 7 human gastric carcinoma cell lines and 18 biopsy specimens from patients with gastric carcinoma. Six out of seven human gastric carcinoma cell lines and twelve of eighteen tumor tissues showed higher levels of Sp1 mRNA comparing to that found in normal mucosa. Increased expression of Sp1 protein was found in human thyroid tumors (Russo et al., (2002) BMC Cancer 2:35). Increased ploidy is related to the aggressive biological behavior neoplastic cells. When the human acute promyelocytic leukemia cells HL60 was treated with 40nMl, 25-Dihydrovitamin D3, a near-tetraploid subline HL60-40A was developed. It exhibited increased ploidy, more aggressive neoplastic behavior, and an increase in Sp1 transcription factor activity. (Studzinski et al. (1996) Cancer Res. 56:5513-21).

### 5. EXAMPLES

This invention is further illustrated by the following examples, which should not be construed as limiting. The contents of all references, patents and published patent applications cited throughout this application are hereby incorporated by reference. Nucleotide and amino acid sequences deposited in public databases as referred to herein are also hereby incorporated by reference.

5.1 Derivation of Cell Strain MSU-1.0 and Malignantly Transformed Derivative MSU-1.1.

In order to identify the molecular changes occurring with malignant transformation, closely matched lines of transformed and untransformed cells were developed. The use of closely matched cell lines permits correlation of the observed differences between the two states with oncogenic transformation and not other properties such as tissue or cell-type specifity. Ideally, such closely matched cell lines correspond to a particular immortalized human cell line and malignantly transformed variant thereof obtained in culture. Many reports of malignant transformation in culture have been made (see e.g., Kakunaga (1978) Proc. Natl. Acad. Sci. (USA) 75: 1334-1338). However, these reports appear to have shown an unintended artifact. Accordingly, a strategy was developed for obtaining closely matched transformed and untransformed cell lines that is based upon working hypothesis that carcinogenesis is a process by which normal cells, over years of sequential clonal selection for various transformed characteristics, ultimately give rise to a cell that has acquired all the changes necessary to be malignant, and that the transformation process is the result of a gain of function by dominant-acting genes (oncogenes) and/or the loss of function of tumor suppressor genes.

In particular, this approach was applied to human fibroblasts in culture using the following steps: (1) transfection of normal cells with oncogenes known to be active in human or animal fibroblastic tumors in order to identify the phenotypic changes that expression of such genes confers on cells; (2) use of such characteristics to identify and/or select for cells that have acquired such transformed characteristics as a result of carcinogen treatment; (3) development of cells that have acquired an infinite, or at least greatly extended, life span in culture, because finite life span human fibroblasts in culture can only undergo 2-3 sequential clonal selections before senescing, and malignant transformation of human cells requires more than 2-3 genetic changes; and (4) distinguishing human fibroblasts that have become malignantly transformed from those merely altered in morphology etc., by requiring that such cells form sarcomas when injected subcutaneously into athynic mice.

A diploid human fibroblast cell line designated LGI from a foreskin-derived cell line obtained from a normal newborn was utilized. This cell line spontaneously turned on its telomerase gene, giving rise to an infinite life span diploid cell strain, designated MSU-1.0 (see FIG. 1). Transfected LGI cells with a v-myc gene, along with the gene for resistance to G418, selected cells for drug resistance, and grew them to the end of their life span. The majority of the descendants of an original transfectant, probably all but one cell, retained their finite life span and senesced on schedule. However, one cell (designated MSU-1.0) turned on its telomerase gene. Escape from senescence cannot have been conferred directly by unregulated expression of the v-myc because the vast majority of progeny of the drug-resistant clone from which MSU-1.0 cells arose, senesced on schedule, i.e., at the same time as the vector controls. Thereby succeeded in generating a lineage of human fibroblasts culminating in cell strains capable of forming sarcomas in athymic mice.

MSU-1.0 cells have maintained a diploid karyotype for over 125 population doublings since crisis and exhibit normal growth control. They express telomerase, but are not anchorage independent, nor growth factor independent. A faster growing, spontaneous variant cell strain, designated MSU-1.1, overgrew the MSU-1.0 culture. These MSU-1.1 cells have a stable, near-diploid karyotype composed of 45 chromosomes, including two marker chromosomes. Neither MSU-1.0 nor MSU-1.1 cells can form tumors in athymic mice. MSU-1.1 cells grow moderately well without exogenous growth factors and form very small colonies in agarose. MSU-1.0 cells, like normal fibroblasts, do not grow under these conditions.

5.2. Malignant Transformation of MSU-1. 1 Cells

First determined if MSU-1.1 cells could be converted into malignant cells by transfection of a Ras oncogene in a vector engineered for overexpression (see Hurlin, et al., (1989) Proc. Natl. Acad. Sci. (USA). 86:187-191; Wilson (1990) Cancer Res. 50: 5587-5593; and Fry, et al., (1990) Oncogene 5: 1415-1418). Independent focus-derived, morphologically-transformed H-Ras, N-Ras, or K-Ras oncogene-transfectants overexpressed Ras oncoproteins, grew to high saturation densities, formed large colonies in soft agar at a high frequency (anchorage independence), and were growth factor independent. The majority of the strains formed progressively growing sarcomas at every site of subcutaneous injection into athymic mice. The tumors reached 1 cm in diameter in <4 wk, were poorly differentiated, invasive fibrosarcomas or round cell sarcomas with a high mitotic index. The cells from these tumors maintained the same stable karyotype seen in parental MSU-1.1 cells, indicating that with high expression of mutant ras, genetic instability is not required for malignant transformation (Hurlin (1989) Proc. Natl. Acad. Sci. (USA) 86:187-191). Overexpression of Ras appears to be necessary because MSU-1.1 cells transfected with these oncogenes in vectors that allowed expression at the normal level did not become malignant (Hurlin, et al., (1989) Proc. Natl. Acad. Sci. (USA) 86:187-191; and Wilson (1990) Cancer Res. 50: 5587-5593).

Next it was determined if MSU-1.1 cells could also be malignantly transformed by chemical carcinogens. Cells were exposed for 30 min. to BPDE at does that lowered cell survival to ˜50% of the control, and after 7 days, assayed for focus formation. A dose-dependent increase in focus-forming cells was observed. No foci were seen in the controls. A high percentage of the cell strains clonally-derived from independent foci formed colonies in soft agar at a high frequency, and proliferated rapidly in medium lacking growth factors. Such strains formed malignant tumors at all the sites of injection into athymic mice with a short latency. However, unlike the Ras oncogene-transformed strains, the carcinogen-transformed cells exhibited chromosomal changes, in addition to the two stable marker chromosomes characteristic of MSU-1.1 cells (see Yang et al., (1992) Proc. Natl. Acad. Sci. (USA) 89: 2237-2241). Cells derived from the tumors exhibited the same chromosomal changes as the cells originally injected, but no additional changes in karyotype. All the focus-derived strains that formed progressively growing fibrosarcomas exhibited a high degree of anchorage independence and growth factor independence. Focus-derived cell strains with a lower degree of anchorage independence and lower growth factor independence took longer to form tumors. However, the kinds of tumors resembled the faster-growing tumors, and when cell strains derived from tumors that exhibited a long latency were returned to culture and re-assayed, they now formed tumors with a very short latency.

5.3. Malignant Human Cell Lines

More than seventy five independently-derived human cell strains have become tumorigenic as a result of being transfected with an oncogene (Hurlin et al., (1989) Proc. Natl. Acad. Sci. (USA) 86:187-191; Wilson (1990) Cancer Res. 50: 5587-5593; Fry et al., (1990) Oncogene 5: 1415-1418) or as a result of a single exposure to a carcinogen, followed by identification of cells that exhibit characteristics of transformed cells, e.g., by the ability to form foci on a monolayer (Yang et al., (1992) Proc. Natl. Acad. Sci. (USA) 89: 2237-2241; Reinhold, et al., (1996) Int. J. Rad. Biol. 69: 707-715; O'Reilly et al., (1998) J. Cell Biol. 150: 577-584; Boley et al., (2000) Cancer Res. 60: 4105-4111). Transformation assay using carcinogens is quantitative, i.e., cells exhibit a dose dependent increase in the frequency of foci. A high percentage of the cell strains derived from these foci form malignant tumors in athymic mice. Using this system, various oncogenes and suppressor genes involved in the process, including a novel suppressor gene, ST7 were identified (see Qing et al., (1999) Oncogene 18: 335-342)

In addition, use was made of fibrosarcoma cell lines derived from patients' fibrosarcomas (e.g., HT1080, NCI, 8387, VIP-FT, and SHAC) as reference standards. Such cells form fibrosarcomas in athymic mice with a short latency, replicate without exogenous growth factors, lack the typical spindle cell morphology in culture, and form large colonies in agarose and distinct foci on a monolayer of normal cells. They will be used for studies of the ability of Sp1/Sp3 ribozymes to stop the growth of human tumors.

5.4. Regulation of MET & Sp1 in Transformed Human Fibroblasts

In this example, transcriptional up-regulation of the c-MET gene in human fibrosarcomas by transcription factor Sp1 is detected. Hepatocyte growth factor (HGF) and its receptor (MET) have been implicated as playing a role in the malignant progression of human fibrosarcomas (see Vande Woude et al., (1997) Ciba Found. Symp. 212: 119-130, 130-142, 148-154). As part of the studies to determine what genetic and non-genetic changes are required for the malignant transformation of human fibroblasts, expression of HGF and MET in non-tumorigenic and tumorigenic cells of the MSU1 lineage, as well as in the human fibrosarcoma cell lines derived from patients, were examined. A c-MET protein was found to be overexpressed in four out of five of the cell lines derived from patients' fibrosarcomas and in all seven malignant cell lines derived from tumors formed in athymic mice by carcinogen-transformed MSU1.1 cells. Comparative Southern blot analysis of the c-MET gene revealed that this was not the result of gene amplification, strongly suggesting that transcriptional regulation plays a major role in c-MET overexpression. Indeed, studies by Liu ((1998) Gene 215: 159-169) and Seol and Zamegar ((1998) Biochim. Biophys. Acta 1395: 252-258) show that there are multiple Sp1 binding sites in the c-MET promoter region (−233 to −68) and that the Sp1 binding sites are responsible for most of the c-MET promoter activity.

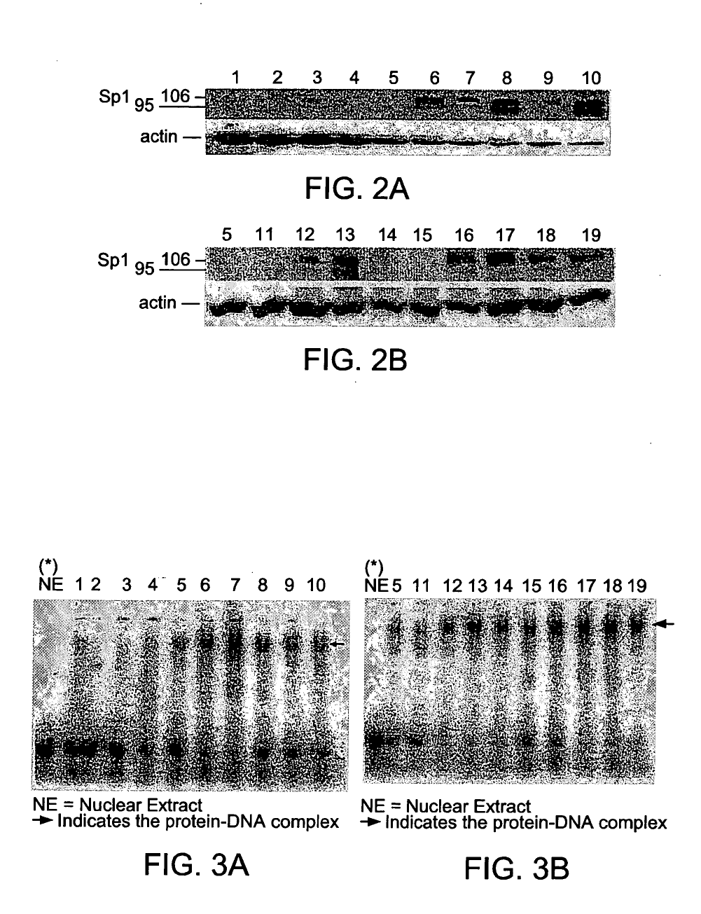

To test the hypothesis that overexpression of c-MET in human fibrosarcoma-derived cells results from transcriptional up-regulation by Sp1, we examined the level of Sp1 expression in the above cell lines by Western blot analysis. FIG. 2 shows Western blot analysis of Sp1 protein expression in human fibroblasts ((a) SP1 expression in normal human fibroblasts (lanes 1-5) and in human fibrosarcoma-derived cell lines (lanes 6-10)) and in cells of the MSU1 lineage ((b) lane 5: LG1; lane 11: MSU-1.0, lane 12: MSU-1.1; and lanes 13-19: carcinogen-transformed MSU-1.1 cells). SP 1 was overexpressed in three out of five human fibrosarcoma cell lines derived from patients' tumors and five out of seven malignant cell lines derived from tumors formed in athymic mice by carcinogen-transformed MSU1.1 cells. Normalization of the relative amount of Sp1 total protein and of actin loaded on the gel (see Tables 1 and 2 below), slightly lowered the amount of Sp1 protein expressed in the fibrosarcoma patients' cells lines, but did not alter the fact that the majority of the malignant cell lines overexpress Sp1 protein. Table 1 shows comparative levels of expression of Sp1 protein in normal fibroblast and corresponding reference cell lines.

TABLE 1

| Cell line or | | Relative | Relative |
|---|---|---|---|
| Strain | Tumorigenicity | Sp1 Level^{a} | Sp1 Level^{b} |
| 15 | − | 1 | 1 |
| L80 | − | 1-2 | 1-2 |
| SL85 | − | 3-4 | 3 |
| SL89 | − | 1 | 1 |
| LG1 | − | 1 | 1 |
| SHAC | + | 16 | 22 |
| NCI | + | 2 | 3 |
| HT1080 | + | 17 | 25 |
| 8387 | + | 3 | 5 |
| VIP: FT | + | 17 | 23 |

^{a}Normalized to total Protein
^{b}Normalized to amount of actin

Table 2 shows comparative levels of expression of Sp1 protein in the MSU-1 lineage.

TABLE 2

| | Tumori- | | Relative Sp1 | Relative Sp1 |
|---|---|---|---|---|
| Cell line or Strain | genicity | Carcinogen | Level^{a} | Level^{b} |
| LG1 | − | — | 1 | 1 |
| MSU-1.0 | − | — | 1 | 1 |
| MSU-1.1 | − | — | 6 | 5 |
| LE10-6A/SB1 | + | BPDE | 17 | 15 |
| DY6: 16.4C5/ST2 | + | BPDE | 4 | 6 |
| SB64.A2C-5/SB2 | + | MNU | 1 | 2 |
| SB22.B1A-1/SF2 | + | MNU | 13 | 17 |
| MW7.3A2/SB1 | + | y-Rays | 18 | 16 |
| MW17.2.4/SFT2 | + | y-Rays | 10 | 8 |
| SOR50J(5)/SB1 | + | y-Rays | 11 | 14 |

^{a}Normalized to total Protein
^{b}Normalized to amount of actin

To investigate the molecular reasons for the observed MET overexpression, electrophoretic mobility shift assays (EMSA) were carried out using a 38 nucleotide DNA sequence from the c-MET promoter and nuclear extracts from various cell strains/lines. Equal amounts of nuclear extract protein from each cell line were incubated, together with 32P-end-labeled double stranded oligonucleotides spanning region −156 to −119 of the c-MET promoter, which contains two Sp1/Sp3 binding sites.

The results of these experiments are shown in FIGS. 3A-C. A marked shift (upper band in FIGS. 3(a) and 3(b) was observed in all tumor cell lines tested (lanes 6-10 and 13-19). MSU-1.1 (lane 12), the immediate parental cell strain for the tumorigenic cell strains in lanes 13-19, also exhibits the shift. LGI (lane 5) and MSU-1.0 (lane 11), the immediate parental cell strains of the MSU-1.1 cells, exhibit a very low DNA shift, similar to that of the other normal fibroblast lines (lanes 1-4). These data indicate that all the malignantly transformed cells and the MSU-1.1 cells have higher levels of protein(s) that bind to a 38 nucleotide fragment of the MET promoter.

5.5. Sp1/Sp3 Transcription Factor Decoy Studies

The fact that three of the 13 cell strains/lines that exhibited a shift in the EMSA (FIG. 3) did not exhibit Sp1 upregulation suggests that the shift observed was not necessarily the result of Sp1/Sp3 DNA binding. To investigate whether it was related to the binding of an Sp1-like protein that does not share the antigenicity of Sp1, a transcription factor decoy (TFD) technique was used to determine whether inhibiting the Sp1 and/or Sp3 protein binding to the c-MET promoter would lead to decreased c-MET expression.

A double-stranded DNA decoy (5′ATTCGATGGGGCGGGGCGAGC-3′) (SEQ ID NO: 12) with the consensus Sp1/Sp3 binding sites was synthesized and also a control composed of mutated Sp1/Sp3 binding sequence (5′ATTCGATGGTTAAGTGCGAGC-3′) (SEQ ID NO:13) as phosphorothioates, which are highly resistant to nucleases. The decoy or the mismatch control was delivered into cells using a liposomal transfection technique. Using time course experiments and transient transfection, evaluated the amount of c-MET protein by Western blotting in LGI, a normal human fibroblast cell line with low levels of c-MET and Sp1 protein, and in HT1080, a fibrosarcoma cell line with high levels of the c-MET and Sp1 proteins.

The amount of the 170 kDa c-MET precursor, 12 hr after treatment of the cells with decoy (TFD), was dramatically reduced (80-90%), along with a 50% decrease in the 145 kDa mature c-MET β-chain protein. The inhibition lasted for at least 24 hr. In contrast, the level of an unrelated control protein (actin) remained constant. Cells treated with the mismatch control DNA exhibited no change in c-MET or actin protein levels. The inhibition was seen most clearly in the HT1080 cells.

These data showed that overexpression of Sp1 plays an important role in the transcriptional up-regulation of c-MET gene expression in human fibrosarcomas. Four cell strains/lines did not exhibit high Sp1 protein levels when evaluated by Western blotting, but three of the latter exhibited an electrophoretic mobility shift with the 21 nt fragment of the MET promoter containing the Sp1/Sp3 binding sequence. The high level of MET protein in the cells correlates with the EMSA results.

5.6. Construction of Chimeric U1snRNA/Ribozyme with Antisense Sp1 and Sp3 Sequences

To determine whether Sp1 could be down regulated by other means, an Sp1 specific ribozyme was constructed and investigated for its ability to block the formation of tumors by fibrosarcoma cell lines. To construct such a ribozyme, the parent vector pUl (provided by Dr. Laterra of Johns Hopkins University. see Abounader et al., (1999) J. Natl. Cancer Inst. 91: 1548-1556) was used. This ribozyme was derived from wild-type U1snRNA as described (see Montgomery and Dietz (1997) Hum. Mol. Genet. 6: 519-525).

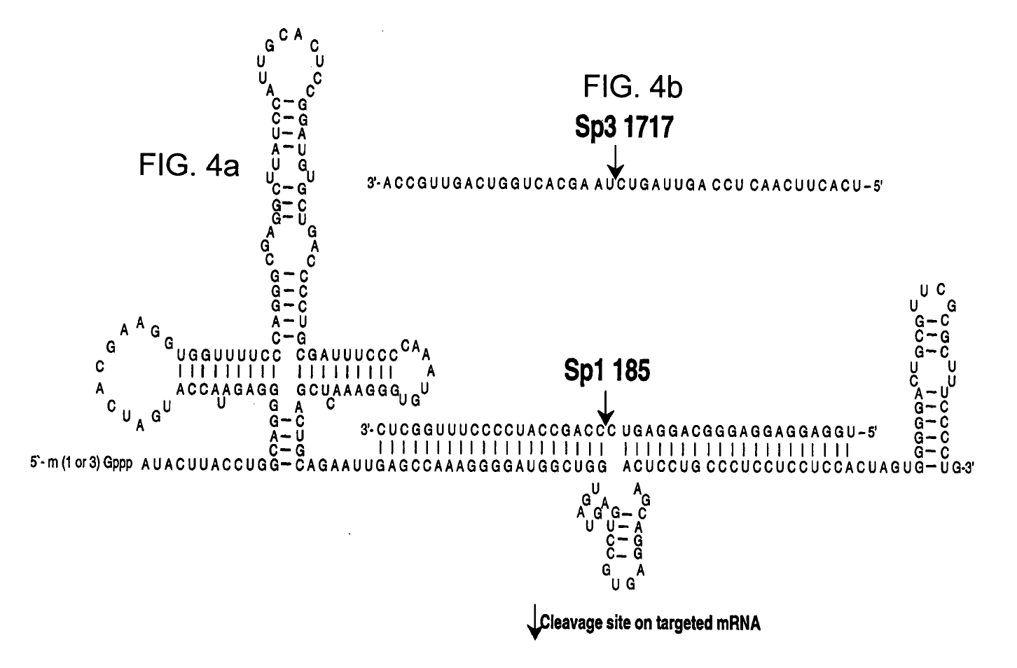

The following strategy was used to design chimeric constructs with antisense sequence against Sp1 mRNA. First, all the ‘GUC’ sequences in the Sp1 mRNA were identified. The shape of the ribozyme generated for each Sp1 ‘GUC’ sequence was analyzed by a computer algorithm (citation to ribozyme/antisense-target algorithm) to identify the theoretically most suitable. With C of ‘GUC’ as the center, and 20 nucleotide extending on either side of it, the sequence was converted into an anti-parallel sequence (antisense) (see FIG. 4A). The 22-nt consensus hammerhead ribozyme sequence (CUG AUG AGC CCG UGA GGA CGA A) was inserted into the middle of that anti-parallel sequence, replacing the G opposite the C of the ‘GUC’ sequence, and the U1snRNA sequences added onto each end and the resulting Sp1 ribozyme mRNA was analyzed by the Mulfold (citation for this algorithm) program, which predicts RNA structures, to determine whether the construct provided maximal preservation of the U1snRNA stem loops, the ribozyme secondary structure, and the accessibility of the antisense sequence to the targeted Sp1 mRNA. Using this strategy, the chimeric U1snRNA/ribozyme shown in FIG. 4A, with antisense Sp1 sequence which cleaves the Sp1 mRNA at the position 185 (according to GenBank Accession #AJ272134) (which corresponds to position 244+x of the Sp1 mRNA according to GenBank No. XM_{—}028606) proved to be theoretically the best ribozyme generated and was used for studies on Sp1. (FIG. 4B shows the corresponding theoretically most suitable construct for an Sp3 ribozyme, which was used for studies of Sp3.)

The antisense construct incorporates the enhancing features of both U1snRNA and ribozyme activity. The U1snRNA, which flanks the ribozyme sequence, is an essential component of the splicosome complex and is stable and abundant in the nucleus of mammalian cells. As a delivery vehicle, the U1snRNA is considered to enhance the stability of the hammerhead ribozyme by conferring resistance to exonucleases. The hammerhead ribozyme used in the construct has the capacity to catalytically cleave a phosphodiester bond intermolecularly in a sequence-specific manner (at the 3′ side of a GUC triplet). The advantage of this method over other types of antisense technology is that because of its catalytic capacity as an ‘enzyme’, one ribozyme molecule can cleave multiple targeted mRNAs. Recent data indicates that it can also functions as an antisense mRNA.

5.7. Construction and Function of Sp1 Ribozyme Expression Vector

The chimeric U1snRNA/ribozyme/Sp1 antisense construct (FIG. 4A) was inserted into the pCMV/Bsd (Invitrogen) cassette, which contains the gene for resistance to blasticidin at the BamH I site. This construct is driven by the human U1snRNA promoter. Two cell lines derived from patients' fibrosarcomas (HT1080 and SHAC), and two cell lines, derived from fibrosarcomas formed in athymic mice by injection of MSU-1.1 cells transformed in culture by carcinogens (MW7.3A2/SB1 and PH2MT) were chosen for this study. These four malignant cell lines exhibited Sp1 protein levels 8-18 times higher than that of normal foreskin-derived fibroblast cell line LG 1, from which the MSU-1.1 cells were derived, and four other foreskin-derived fibroblast cell lines from normal newborns. The four malignant cell lines were transfected with this ribozyme expression vector or with an empty vector as a control and transfectants were selected for blasticidin resistance. Drug resistant clones were isolated and expanded.

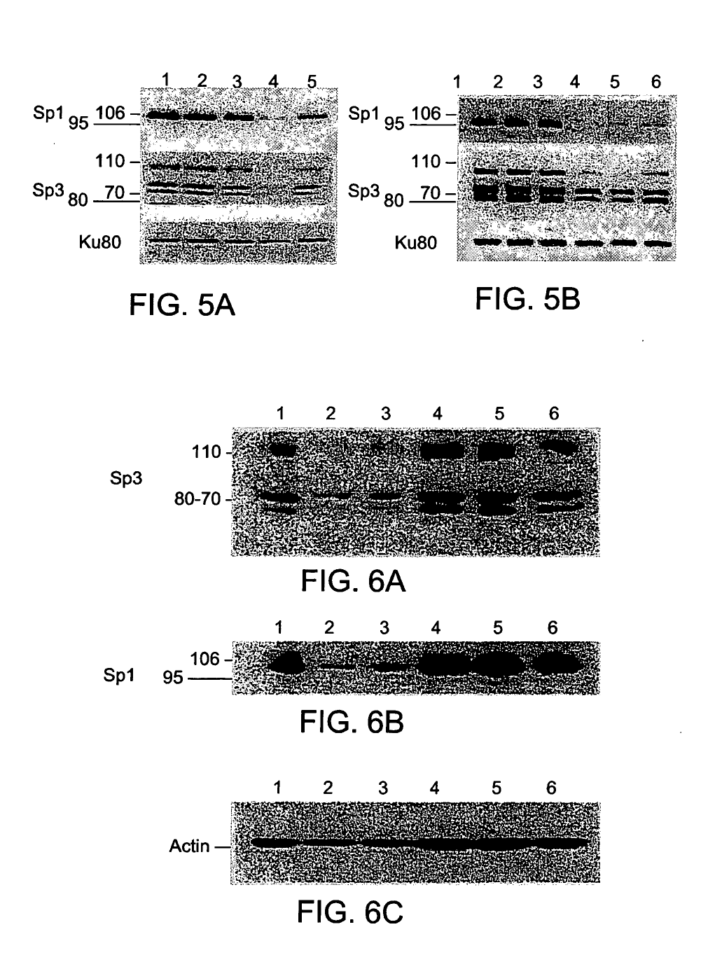

Transfection of HT1080 cells yielded 21 such colonies; the SHAC cell line, 12 colonies; and the MW73A2/SB1 and PH2MT cell lines, ˜50 colonies each. Vector control transfectants were also isolated. The clonal populations were expanded, and their cell lysates screened by Western blotting for the level of Sp1. Two of the 50 clones from the Sp1 ribozyme-transfectants of the PH2MT cell line (FIG. 5A, top half, lanes 4 and 5), and three of the clones of the MW7.3A2/SB1 cell line (FIG. 5B, top half, lanes 4-6) exhibited markedly reduced levels of Sp1 protein (down to 10-15% of that found in their respective control). Three other Sp1 ribozyme-transfectants derived from the PH2MT cell line exhibited a moderate level of Sp1 protein expression. None of the empty vector controls exhibited a reduction in the level of Sp1 (see FIGS. 5A and 5B, lanes 2 and 3). Stripped these Western blots and screened them for the level of Sp3 protein (Bottom half of FIGS. 5A and 5B). The levels of Sp1 and Sp3 proteins were found to be coordinately expressed. If the Sp1 level was high, so too was the Sp3 level. If the Sp1 level was intermediate or low, the Sp3 level was intermediate or low. These results indicate that Sp1 and Sp3 are coordinately regulated.

5.8. Sp1 Ribozyme Depletes Sp2 mRNA and Functional Sp1 Protein

The U1snRNA/ribozyme/Sp1 antisense construct is intended to function as a ribozyme, cutting the target mRNA. It may also function as antisense mRNA by forming a duplex structure with the Sp1 target mRNA and inhibiting translation. To see if the five cell strains in FIGS. 5A and B that expressed lower amounts of Sp1/Sp3 protein had significantly lower levels of Sp1 mRNA, indicating ribozyme activity, they were assayed for amount of Sp1 mRNA using quantitative RT-PCR. Two strains showed decreased levels of mRNA, indicating ribozyme cutting action on mRNA; the other three did not, suggesting that in them, the ribozyme functioned as antisense.

Evidence that the Functional Form of Sp1 Protein is inactivated by the Sp1 Ribozyme. To determine the Sp1 transactivation activity in cell strains with reduced Sp1 protein levels, compared to that in their parental (P) and vector control cell strains (V1, V2), carried out reporter gene (luciferase) assays using a vector with several Sp1 binding sites, chosen because Sp1 protein is subject to post-translational modification, e.g., phosphorylation, glycosylation, etc., and changes in the transcriptional activity of Sp1 have been reported to result from such modifications. The reporter gene vector was transiently transfected into these five cell strains, as well as into the parental cell line and its vector control cell strains. Sp1 transactivation activity in the two transfected PH2MT cell strains was reduced by 90%; that in the three transfected MW7.3A2/SB1 cell strains was reduced by 75%. Accordingly, the Sp1 ribozyme depletes Sp1 protein activity as well as Sp1 nmRNA and protein.

5.8.A. Evidence that the Functional Form of Sp1 Protein Is Inactivated by the Sp1 Ribozyme.

Reporter gene (luciferase) assays were carried out using a promoter with several Sp1/Sp3 binding sites to determine if the Sp1/Sp3 transactivation activity in cell strains with reduced Sp1/Sp3 protein levels compared to that in their parental (P) and vector control cell strains (V1, V2). These were chosen because Sp1 protein is subject to post-translational modification, (e.g., phosphorylation, glycosylation, etc.) and changes in the transcriptional activity of Sp1 have been reported to result from such modifications. The reporter gene vector was transiently transfected into these five cell strains, as well as into the parental cell line and its vector control cell strains. Sp1 transactivation activity in the two transfected PH2MT cell strains was reduced by 90%; that in the three transfected MW7.3A2/SB1 cell strains was reduced by 75%. Accordingly, the Sp1 ribozyme depletes Sp1 and/or Sp3 protein activity as well as Sp1 mRNA and Sp1 and Sp3 protein levels.

5.9. Sp1 Hammerhead Ribozyme Prevents Anchorage Independence and Tumor Formation

The parental cell lines, MW7.3A2/SB1 and PH2MT, two of their empty vector transfectants, and six of their Sp1 ribozyme transfectant cell lines that exhibited reduced levels of Sp1 protein (FIGS. 5A and B) were injected subcutaneously into athymic mice to determine whether they formed tumors. They were also tested for loss of anchorage independence. As shown in Tables 3 and 4, down-regulation of Sp1 and Sp3 protein levels correlated with loss or decreased frequency of tumor formation, increase in length of tumor latency, and decreased anchorage independence. Notably, there was no significant change in the growth rate (doubling time) of the cell lines. The mice that did not form tumors were observed for at least 26 wks.

Table 3 shows the effect of down-regulation of Sp1/Sp3 protein in the malignant human fibroblast cell line MW7.3A2/SB1, derived from a tumor formed in athymic mice by MSU-1.1 cells malignantly transformed in culture by gamma radiation, on the cells' ability to form tumors in athymic mice and colonies in soft agar.

TABLE 3

| | Relative | | | Tumorigenicity in | | | | |
|---|---|---|---|---|---|---|---|---|
| | Level | | Doubling | Athymic Mice | | Anchorage Independence (Colonies | | |
| | of Protein | | Time | Tumors/ | | with Diameters of Specified Size) | | |
| Cell Line Tested | Sp1 | Sp3 | Hours | Sites | Latency^{a (wks)} | <40 μm | 40-120 μm | >120 μm |
| Parental cell line | ++++ | ++++ | 22 | 6/6 | 7 | 72% | 26% | 2% |
| Vector-transfectant 1 | ++++ | ++++ | 22 | 5/6 | 5 | 68% | 25% | 6% |
| Vector-transfectant 2 | ++++ | ++++ | 17 | 6/6 | 5 | 28% | 30% | 42% |
| Sp1-ribozyme | + | + | 23 | 0/6 | NA^{b} | 98% | 2% | 0% |
| transf. 1 | | | | | | | | |
| Sp1-ribozyme | + | + | 24 | 0/6 | NA | 100% | 0% | 0% |
| transf. 2 | | | | | | | | |
| Sp1-ribozyme | + | + | 24 | 0/6 | NA | 99% | 1% | 0% |
| transf. 3 | | | | | | | | |

^{a}Time in weeks for the tumors to reach −1 cm in diameter
^{b}Not determined
^{c}Not applicable

Table 4 shows the effect of down-regulation of Sp1/Sp3 protein in a malignant human fibroblast cell line (PH2MT), derived from a tumor formed in athymic mice by MSU-1.1. cells malignantly transformed in culture by a transfected H-Ras oncogen,, on the cells' ability to form tumors in athymnic mice and colonies in soft agar.

TABLE 4

| | Relative | | | Tumorigenicity in | | Anchorage Independence | | |
|---|---|---|---|---|---|---|---|---|
| | Protein | | Doubling | Athymic Mice | | (Colonies with Diameters of | | |
| | Level | | Time | Tumors | | Specific Size) | | |
| Cell Line Tested | Sp1 | Sp3 | Hours | per Sites | Latency^{a (wks)} | <40 μm | 40-120 μm | >120 μm |
| Parental cell line | ++++ | ++++ | 19 | 6/6 | 6 | 49% | 49% | 49% |
| Vector-transfectant 1 | ++++ | ++++ | 19 | 4/4 | 8-10 | 39% | 56% | 5% |
| Vector-transfectant 2 | ++++ | ++++ | 19 | 6/6 | 6-8 | 34% | 58% | 8% |
| Sp1-ribozyme transf. 7 | +++ | +++ | ND | 6/6 | 7 | ND | ND | ND |
| Sp1-ribozyme transf. 3 | +++ | +++ | ND | 6/6 | 7-9 | ND | ND | ND |
| Sp1-ribozyme transf. 5 | ++ | ++ | ND | 3/8 | 12-26 | ND | ND | ND |
| Sp1-ribozyme transf. 8 | ++ | ++ | ND | 3/10 | 16-18 | ND | ND | ND |
| Sp1-ribozyme transf. 1 | + | + | 19 | 0/6 | NA^{c} | 90% | 10% | 0% |
| Sp1-ribozyme transf. 2 | + | + | 37 | 0/6 | NA | 93% | 7% | 0% |

^{a}Time in weeks for the tumors to reach −1 cm in diameter.
^{b}Not determined.
^{c}Not applicable

Accordingly, these results show that the Sp1 hammerhead ribozyme prevents malignant cancer activities of multiple independently derived malignant fibrosarcoma cell lines.

5.10. Construction of an Sp3 Hammerhead Ribozyme and Expression Vector

Using the same strategy described for SPI above, designed and constructed the chimeric U1sn-RNA/ribozyme construct sequence shown in FIG. 4B. PH2MT cells were transfected with this vector and selected for drug-resistant transfectants. The transfectants were analyzed by Western blotting to determine the level of Sp3 and Sp1 protein.

The data in FIG. 6 show that the Sp3 ribozyme down-regulated not only the level of Sp3 protein, but also Sp1. FIG. 6 shows a Western blot analysis of Sp3 and Sp1 protein expression in PH2MT-transfectants (lane 1, parental cells; lanes 24, U1snRNA/Ribozyme/Sp3 antisense construct transfectants; and lanes 5 and 6, vector-transfectant controls).

To see if these results could have been caused by the Sp3 ribozyme cutting both species of mRNA, compared the antisense sequence of the Sp3 ribozyme with the cDNA sequence of Sp1. There was no homology. (FIG. 4) This result suggests that the promoter of Sp3 has the binding sites common to both transcription factors, and that, therefore, Sp3 can regulate expression of both proteins and that the same is true for Sp1.

5.11. Sp3 Hammerhead Ribozyme Prevents Anchorage Independence and Tumor Formation

Three Sp3 ribozyme transfectants that showed somewhat decreased expression of Sp3/Sp1 were compared with their parental strain and vector controls for ability to form s.c. tumors in mice (see Table 5).

TABLE 5

| | | | Tumorigenicity in | |
|---|---|---|---|---|
| | Relative Level | | Athymic Mice | |
| | of Protein | | Tumors/ | |
| Cell Line Tested | Sp1 | Sp3 | Sites | Latency^{a (wks)} |
| Parental cell line | +++++ | +++++ | 6/6 | 7 |
| Vector-transfectant 2D | +++++ | +++++ | 6/6 | 7 |
| Vector-transfectant 3B | +++++ | +++++ | 6/6 | 7-8 |
| Vector-transfectant 3C | +++++ | +++++ | 4/6 | 9 |
| Sp1-ribozyme transf. 10C | +++ | +++ | 6/6 | 7-10 |
| Sp1-ribozyme transf. 3E | ++ | ++ | 6/6 | 12-21 |
| Sp1-ribozyme transf. 2D | ++ | ++ | 5/6 | 17-26 |

The decrease in expression of Sp1 and Sp3 in these three strains (FIG. 6) was accompanied by a very significant lengthening of the latency period for the tumors that formed from these cells.

Table 5 shows the effect of down-regulation of Sp1/Sp3 protein on the tumorigenicity of fibroblast cell line PH2MT, derived from a tumor formed in athymic mice by MSU-1.1 cells malignantly transformed in culture by transfection of H-ras, on tumorigenicity.

5.12. Characterization of Human Pancreatic Cell Lines

In order to determine whether Sp1/Sp3-inhibition could be used to treat human pancreatic carcinomas as well as human fibrosarcomas, by characterizing eight human pancreatic carcinoma-derived cell lines. Before attempting to down-regulate Sp1 and/or Sp3 protein expression in the pancreatic cancer cell lines by ribozyme vectors found to be effective in down-regulating the protein levels in fibrosarcoma-derived cell lines and blocking their tumor forming ability, tested the ability of 8 pancreatic carcinoma cell lines listed in Table 6, to form carcinomas when injected subcutaneously into mice.

TABLE 6

| | Sp1 Levels^{a} | | Tumors | | |
|---|---|---|---|---|---|
| Cell Code | Log | Conf. | per site | Latency^{a (wks)} | Histology |
| BxPC-3 | + | +++ | 6/6 | 7 | Squamous Carcinoma |
| PANC-1 | +++ | ++++ | 4/6 | 7.4 | Carcinoma^{c} |
| MIAPaCa- | ++++ | ++++ | 6/6 | 7 | Carcinoma^{c} |
| 2 | | | | | |
| HPAF-11 | ++++ | ND^{d} | 4/4 | 4.5 | Adenocarcinoma^{e} |
| Capan-1 | + | + | 6/6 | 3.5 | Adenocarcinoma^{e} |
| Capan-2 | ++++ | +++ | 6/6 | 3.5 | Adenocarcinoma^{e} |
| AsPC-1 | ++++ | − | 6/6 | 4 | Carcinoma^{c} |
| CFPAC-1 | ++++ | ++ | 6/6 | 4 | Adenocarcinoma^{e} |

^{a}Determined by Western blotting of lysates from cells in log phase growth of confluent.
^{b}Time required for tumors to reach 1 cm in diameter.
^{c}Highly anaplastic carcinoma.
^{d}Not determined
^{e}High grade adenocarcinoma

The results are shown in the right half of Table 6. All eight formed carcinomas at virtually all sites of injection with a short latency. Four of the cell lines formed classic adenocarcinomas; three formed highly anaplastic carcinomas; and one (BxPC-3) formed squamous carcinomas.

The Sp1 levels in these cell lines during log-phase growth and in confluence were then assayed. Lysates from normal human fibroblasts in log-phase growth, which exhibit low levels of Sp1 protein, and lysates from human fibrosarcoma-derived cell line HT1080 in log-phase growth, which exhibits high levels of Sp1 protein, were used as standards. The majority of the cell lines derived from human pancreatic carcinomas expressed high levels of Sp1, left half of Table 6

5.13. Sp1 Ribozyme Down-regulates Sp1 Protein in Pancreatic Carcinoma Cell Lines

In order to determine whether the Sp1 hammerhead ribozyme could be used to deplete Sp1 levels in human pancreatic carcinoma cell lines, CFPAC-1 and PANC-1 were transfected with a vector carrying the Sp1 ribozyme, or an empty vector as a controls. Puromycin-resistant transfectants were selected, and lysates were prepared from 15 such clones and assayed by Western blotting for expression of Sp1 protein.

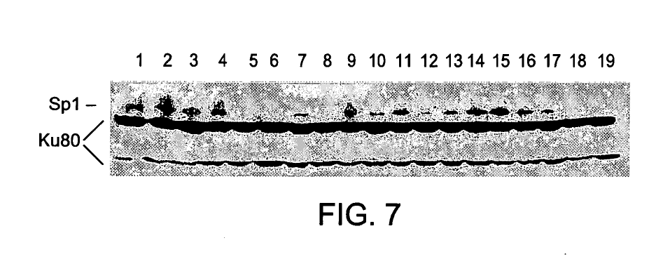

FIG. 7 shows that the Sp1 ribozyme eliminated or significantly decreased expression of Sp1 in 11 of the 15 transfectants assayed. To see if the results shown in FIG. 7 were reproducible, fresh lysates were prepared from these cell lines and similarly analyzed for expression of Sp1. The results were identical. These 15 cell lines were also assayed by Western blotting for expression of Sp3. As expected, the expression levels of Sp3 and Sp1 were coordinately down regulated by the transfection of the Sp1 ribozyme. Accordingly, it appears that Sp1 hammerhead ribozyme can be used to reduce levels of Sp1/Sp3 in human pancreatic carcinomas.

5.14. Animal Model for the Treatment of Human Pancreatic Carcinoma Using Cell Lines Expressing DsRed

In order to carefully monitor the treatment of carcinomas with Sp1/Sp3-targeted ribozymes or other recombinant Sp1/Sp3-targeted inhibitory agents, a fluorescent protein-based marking system was developed. This system was developed in two stages: first, a red fluorescent protein marker was engineered into pancreatic carcinoma-derived tumors; and then a second complementary fluorescent protein marker was engineered into a vector designed for the delivery of the recombinant Sp1/Sp3-inhibitory agent. The overlap in expression of the two complementary fluorescent proteins provides an indication of the extent to which the tumor is effectively treated with the recombinant Sp1/Sp3-inhibitory agent.

The Green Fluorescent Protein (GFP) gene has been modified to code for a green protein with a slightly red shifted excitation and emission wavelengths but which has a much longer fluorescent half-life and a much enhanced emission signal, Enhanced Green Fluorescent Protein (EGFP). The red fluorescent gene, which was isolated from coral, has been similarly modified. The gene that codes for the enhanced red fluorescent protein is referred to by BD-Clontech as DsRed. The DsRed gene is used here as a label for tumor cells that will be assayed for tumor formation in athymic mice because the autofluorescence and light scattering of the mouse is less for red fluorescence than green fluorescence. In this wording example, the pancreatic cancer cells carry the DsRed gene and the virus carries the EGFP gene, but the fluorescent markers may be reversed and other markers may be substituted for each. In some experiments to obtain optimal results, may choose to reverse the labels and the system is generally functional in either such orientation of the fluorescent labels.

This fluorescent-based system is used to further demonstrate that the Sp1/Sp3-inhibitory ribozymes can lower the level of Sp1/Sp3 protein in human pancreatic carcinoma-derived cell lines that express Sp1/Sp3 at high levels, and that when the level of expression is significantly reduced, the ability of the cells to form subcutaneous tumors and orthotopic tumors as well as the metastatic spread from orthotopic tumors in athymic mice will be reduced or blocked completely.

The fact that parental carcinoma-derived cell line PANC-1 and adenocarcinoma-derived cell line CFPAC-1 are tumorigenic was demonstrated (see Table 6) and also the fact that they exhibit anchorage independence was shown. As shown in FIG. 7, several Sp1 ribozyme-transfected derivatives of these parental cell strains that exhibit a markedly reduced level of Sp1 protein, as judged by Western blotting, have been identified. This series of derivative cell strains is tested for the ability to form subcutaneous tumors in athymic mice and also for their ability to form large-sized colonies in agarose (anchorage independence). The ability of the Sp1 and/or Sp3 ribozyme to decrease expression of Sp1/Sp3 protein in other patient-derived pancreatic carcinoma cell lines listed in Table 6 is also tested using this system.

Before transfecting the human pancreatic carcinoma cell lines the gene for DsRed fluorescence carried on a vector from BD Clonetech (Palo Alto, Calif.) was first introduced, along with the gene coding for resistance to G418 as the selectable maker. For each of the cell lines, G418-resistant transfectants are isolated and pooled together with their siblings, producing once again six separate cell lines of pancreatic carcinoma-derived cells that will fluoresce red when illuminated with the proper wavelength. Before being used as recipients of Sp1 ribozyme transfection, these six cell lines are assayed for the ability to make subcutaneous tumors in athymic mice and for anchorage independence to be certain that they still possess those properties of carcinomas. These “DsRed” cell lines are transfected with the Sp1 ribozyme vector that contains the gene coding for resistance to puromycin as the selectable marker. Drug resistant transfectant cell strains are isolated, and assayed for expression of Sp1/Sp3 protein by Western blotting (as in FIG. 7). The cell strains showing the greatest decrease in Sp1 and/or Sp3 protein will be tested for their ability to form tumors when injected subcutaneously into athymic mice, and for their anchorage independence (ability to form large sized colonies in agarose). In all of the above experiments, the parental cell line, and a series of cell strains derived from transfection of the empty vector, are used as controls.

Using this system, it is shown that cells that express low levels of Sp1 and/or Sp3 will not form tumors or will form tumors at a much lower frequency. Cells that express intermediate levels of Sp1 and/or Sp3 form tumors after a longer latency and that the frequency of such tumors will be reduced. Based on the results obtained using the human fibrosarcoma cells in which expression of Sp1 and/or Sp3 protein had been significantly reduced by the ribozymes, the pancreatic carcinoma cells expressing low levels of Sp1 and/or Sp3 also do not form colonies in agarose or make only very small ones, and the cells with intermediate levels of Sp1/Sp3 form intermediate sized colonies. Later, cell lines PANC-1 and CFPAC-1 are similarly transfected with the gene for DsRed fluorescence in order to be available for studies using this marker approach.

The fluorescence-based system of the invention is employed using both a subcutaneous tumor model system and an orthotopic tumor model, as each has its own advantages. In particular, when testing human tumor cells for growth in immunosuppressed mice, most place them into the tissue from which they would have been located if derived from a mouse tumor, that is, orthotopically. However, it has frequently been found that injection of tumor cells under the skin is a suitable site to test cells for their ability to form tumors, even though it is not “anatomically correct”. A strong advantage of injections just under the skin, i.e., subcutaneously (s.c.) is that this allows one to easily monitor tumor growth by measuring the tumors directly on a regular basis. When the eight pancreatic carcinoma cell lines described in Table 6 were injected subcutaneously, it was found that their growth rate could readily be monitored and was morphologically consistent with pancreatic carcinomas, i.e., high grade adenocarcinomas, highly anaplastic carcinomas, and squamous carcinomas.

To determine whether an engineered virus (described below) carrying a recombinant Sp1/Sp3-inhibitory agent, such as an Sp1/Sp3 ribozyme, can slow or inhibit tumor growth, s.c. tumors are useful because the virus can be directly injected into the tumor with certainty and its growth monitored directly. Therefore utilize s.c. injection to test the rate of tumor growth of pancreatic cells that exhibit down-regulation of Sp1 and/or Sp3 protein after transfection with Sp1 or Sp3 ribozymes. Because, as indicated above, the tumor-derived cell lines used contain the DsRed gene, and can simultaneously determine the fluorescent signal from the tumor cells. Having both types of measurements for the s.c. tumors allow one to validate the use of the measurement of the fluorescent signal as a means of measuring pancreatic tumor growth. This allows us later to use the fluorescent signal to monitor orthotopically implanted pancreatic tumor cells. (See below). These tumor experiments are especially significant because they demonstrate directly that down-regulation of Sp1 and/or Sp3 interferes with tumor formation.

Orthotopic tumor experiments are used to more closely mimic the situation of a patient with pancreatic cancer (i.e., an in situ human pancreatic carcinoma). The technique described by Bouvet et al., ((2002) Cancer Res. 62: 1534-1540) is used. Accordingly, pancreatic tumors grown s.c. in nude mice are harvested at the exponential growth phase and resected under aseptic conditions. Necrotic tissues are cut away, and the remaining healthy tumor tissue is cut with surgical scissors and minced into pieces of about ˜1 mm³ in size in a suitable medium containing penicillin (100 units/ml) and streptomycin (100 pg/ml). For orthotopic surgery, mice are anesthetized as described below. The abdomen is sterilized with alcohol. An incision is made through the left upper abdominal pararectal line and peritoneum. The pancreas is carefully exposed, and three tumor fragments are transplanted on the middle of the pancreas with a 6-0 Dexon (Davis-Geck, Inc., Manati, Puerto-Rico) surgical suture. The pancreas is then returned to the peritoneal cavity, the abdominal wall, and the skin is closed with 6-0 Dexon sutures. These procedures are performed with a dissecting microscope.

Serial sacrifice of a cohort of animals used to follow tumor growth. However, it is advantageous for many reasons to be able to follow the efficacy of tumor treatment on tumor growth and metastasis using living animal hosts. Accordingly, a fluorescent system to monitor tumor growth and treatment in situ was utilized. First, Green Fluorescent Protein (GFP)-marked pancreatic carcinoma cells are injected into the pancreas of athymic mice, as described above, and tumor growth is monitored by recording the GFP fluorescent signal weekly. Then, the pancreatic carcinoma is treated with a DsRed-labeled recombinant vector carrying the Sp1/Sp3-inhibitory agent. Use of this method allows one to determine whether the pancreatic carcinoma cells effectively expressing the recombinant Sp1/Sp3-inhibitory agent vector, and that thereby express low levels of Sp1 and/or Sp3, also fail to form tumors or grow more slowly than control cells.

When performing whole animal imaging experiments, the DsRed fluorescent protein provides a stronger signal than GFP, or EGFP, and, according, the use of the DsRed gene for these studies is particularly useful. As few as 500-1000 DsRed-labeled cells that have been injected into mice s.c. and as few as 104-105 labeled cells in the pancreas (see Hoffman (2002) Lancet Oncol. 3: 546-556) can be detected using this fluorescent protein. By stably expressing the GFP gene in human cancer cells, it is possible to detect the fluorescent signal from the cells growing as a subcutaneously implanted tumor or a deep internal tumors, such as orthotopically implanted pancreatic cancer cells in athymic mice. When colon adenocarcinoma cells labeled with GFP are implanted in the flanks of athymic mice, a progressive increase in intensity of the fluorescent signal correlates with an increase in tumor volume and with tumor weight (see Diehn et al., (2002) Biotechniques 33: 1250-1252 and 1254-1255). Furthermore, the metastatic spread of two different human pancreatic cell lines transplanted into the pancreas of athymic mice as described above evince strikingly different results that mimic what is seen in human patients with pancreatic cancer. In particular, the MIA-PaCa-2 cell line formed tumors relatively slowly in the pancreas, but rapidly metastasized to selective sites including the liver and portal lymph nodes. With this cell line, retroperitoneal lymph node metastases were rare. In contrast, the BxPC-3 cell line rapidly formed a tumor in the pancreas and spread regionally to the spleen and retroperitoneum within 6 wks. Accordingly, both of these cell lines are used in our fluorescence single and double-labeling system.

The ribozyme-expressing cell lines that exhibit downregulation of Sp1 and/or Sp3, but are still able to form tumors when orthotopically transplanted, are examined for changes in the pattern of metastatic spread. Fluorescently labeled, orthotopically transplanted cell lines are examined for metastatic spread by detection of the red fluorescent signal of the cells. The mice are eventually sacrificed, and a necropsy is carried out to locate the red fluorescing metastatic lesions. Lesions are removed and fixed in 1% paraformaldehyde and pathology slides are prepared. The fluorescent signal is stable for 19 months in soft tissue fixed in this way (see Harms et al., (2002) Biotechniques 33: 1197-1200).

5.15. Whole-Animal Imaging

An instrument developed by Lightools (Encinitas, Calif.) is used to record the signals produced by DsRed and EGFP labeled tumor cells growing in athymic mice and to monitor treatment with recombinant Sp1/Sp3-inhibitory agent in real time. This instrument consists of a light tight box with a thermo-electrically-cooled color CCD camera mounted in the top to collect the signal. The box contains a light source with appropriate filters to excite the EGFP molecules and a second light source with appropriate filters to excite only the DsRed molecules. Emission filters for each signal are mounted on a wheel mounted in front of the camera lens so that either can be selected. If both fluorescent signals are present, the images may be acquired separately and superimposed to determine whether the signals come from the same location. With this system, a mouse is placed in the box and the fluorescent signal is recorded and processed by a computer using Image Pro Plus 4.0 software (see e.g., Bouvet et al., (2002) Cancer Res. 62: 1534-1540) or similar software. The mice do not need to be anesthetized because the signal is acquired in times that run from milliseconds to a second or so. This allows quantification of tumor growth by measurements taken on the each animal every few days.

A CCD camera coupled to an appropriate microscope is used to image tissue sections in which the cells express with the EGFP and/or the DsRed protein. The EGFP signal, the DsRed signal, or a yellow signal is seen when cells express both the green and red fluorescent signals.

A dissecting microscope similarly equipped allows detection of small metastatic lesions in animals at the time of necropsy. A flashlight and filters is also available for such use (see Tyas et al., ((2003) Biotechniques 34: 474-476).

Because this technology makes it possible to study Sp1/Sp3-targeted (as well as other) cancer treatments in real time, all of the main cell lines required for our experiments were transfected with the DsRed gene, and select clones of cells that express this protein were expanded and the siblings pooled to produce cell lines again. The eight cell lines derived from this pooling are tested to make sure that they form the same kinds of tumors as the original parental cell lines did (see Table 6), and do so with an equivalent frequency and latency. Whether the cell lines form colonies in agarose at the same frequency as the parental cell lines was then determined, as was expression of Sp1 and Sp3 protein at levels equivalent to their original parental cells. Once these DsRed cell strains have been validated, they are used for all of our studies. This greatly simplifyies the proposed studies, and facilitate comparisons between experiments.

5.16. Anchorage Independence Assay

The ability of cancer cells to form large colonies in agarose is the in vitro assay that correlates best with the ability to form tumors in a susceptible animal host. With human fibroblasts, a correlation >95% is found. It was found that the eight pancreatic tumor-derived cell lines (Table 6) all make large colonies in agarose (>200 μm in diameter). Accordingly, each of the newly created DsRed pancreatic cell lines with down-regulated Sp1 and/or Sp3 was tested for their ability to form colonies in agarose. A large difference, as demonstrated with Sp1 and/or Sp3 down-regulated fibroblasts, would warrant a visual examination of the agarose dishes sufficient to judge that no colonies formed. Where the colonies exhibit only a small reduction in size, a microscope set-up that can scan multiple fields of cells and size the colonies is used. Because this assay requires only 2-3 weeks, it can serve as an efficient method to determine which cell strains are of particular interest.

5.17. Construction of rAAV Vectors (Plasmid pAAV)

In order to deliver the Sp1/Sp3-inhibitory ribozymes efficiently into human pancreatic carcinomas, a viral vector for delivery in athymic mice to subcutaneous or orthotopic tumors was developed. In particular, a family of replication-defective recombinant adeno-associated virus (rAAV) DNA vectors carrying, in various combinations, the Sp1 or Sp3 ribozyme under the control of various promoters and also the genes for Enhanced Green Fluorescent Protein (EGFP) and puromycin-resistance was developed for use in monitoring and selection as described above.

For replication and packaging of a DNA sequence into rAAV particles, the DNA must contain an ITR at each end, and the sequence must be about 4.5kb. This generally allows the vector to carry one or two genes, each under the control of its own promoter.

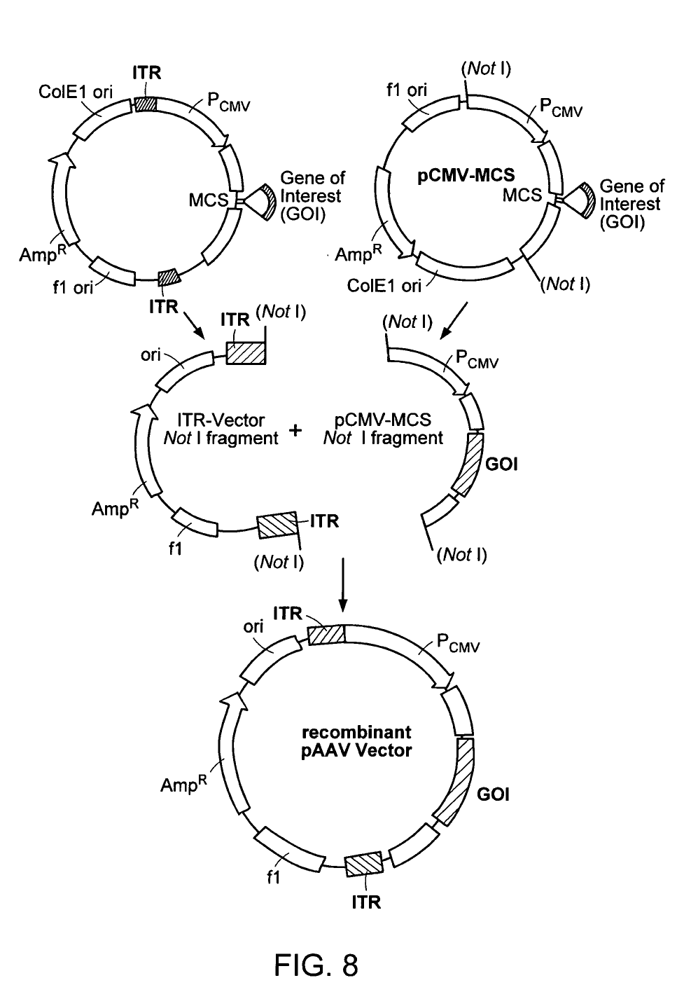

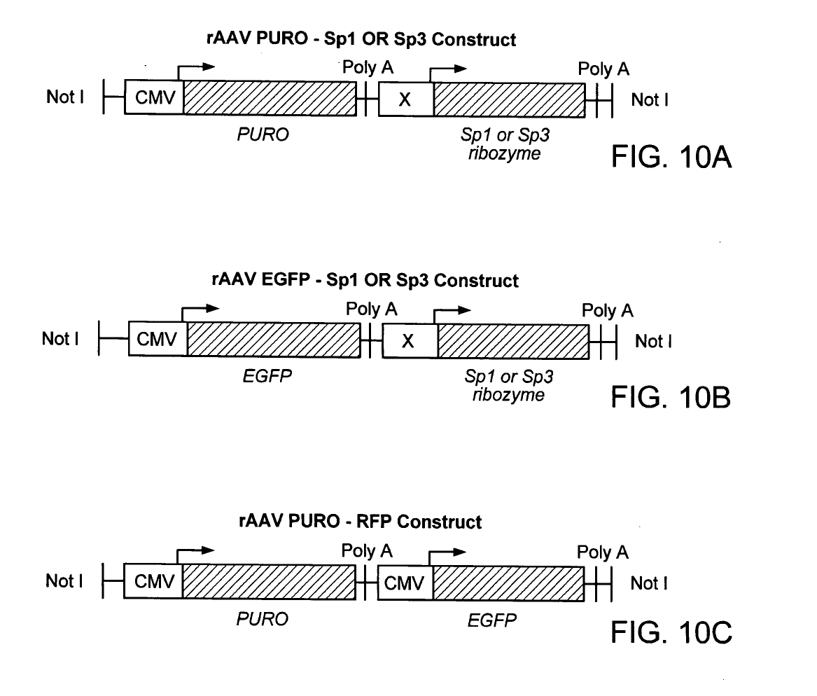

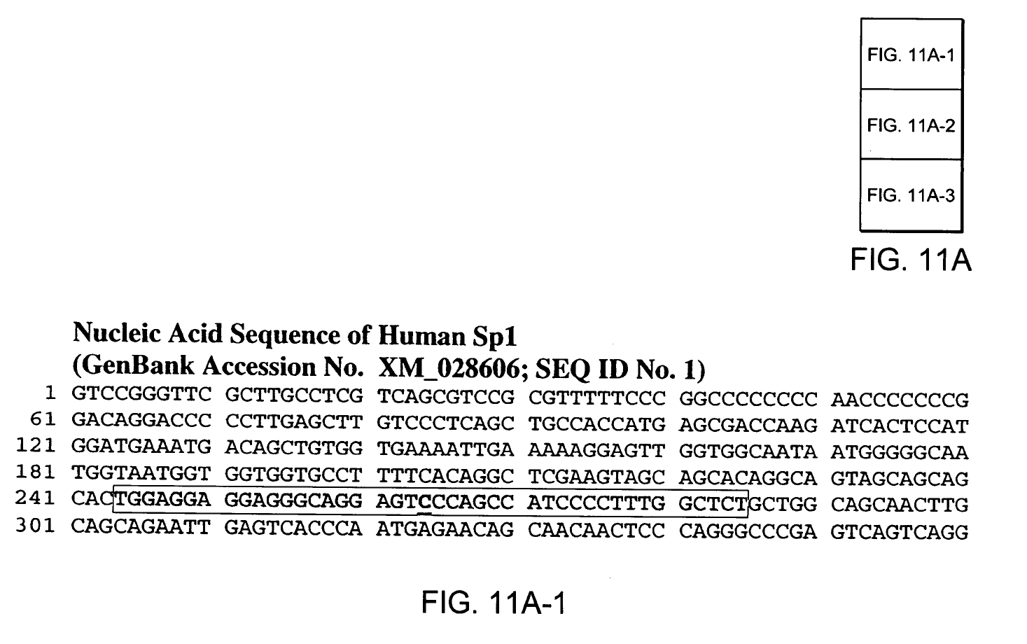

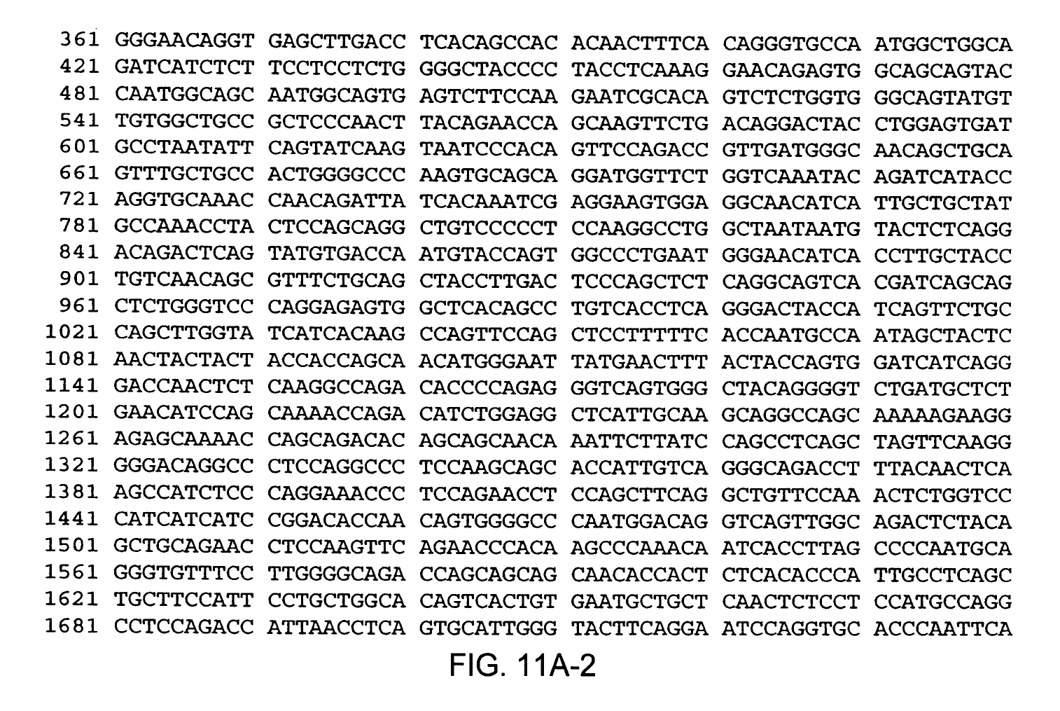

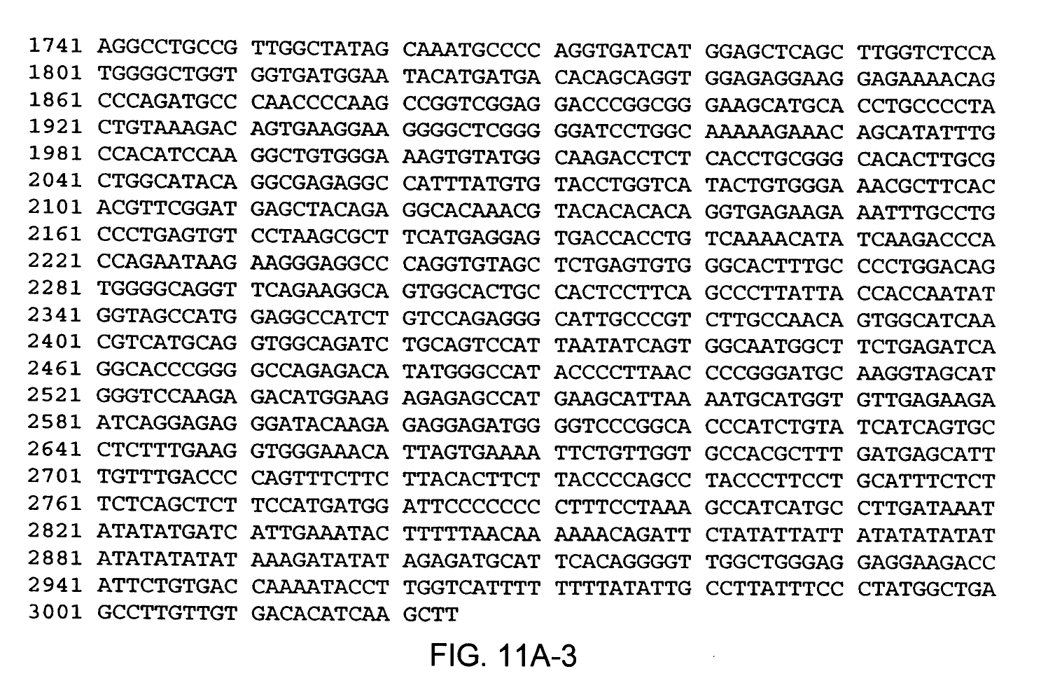

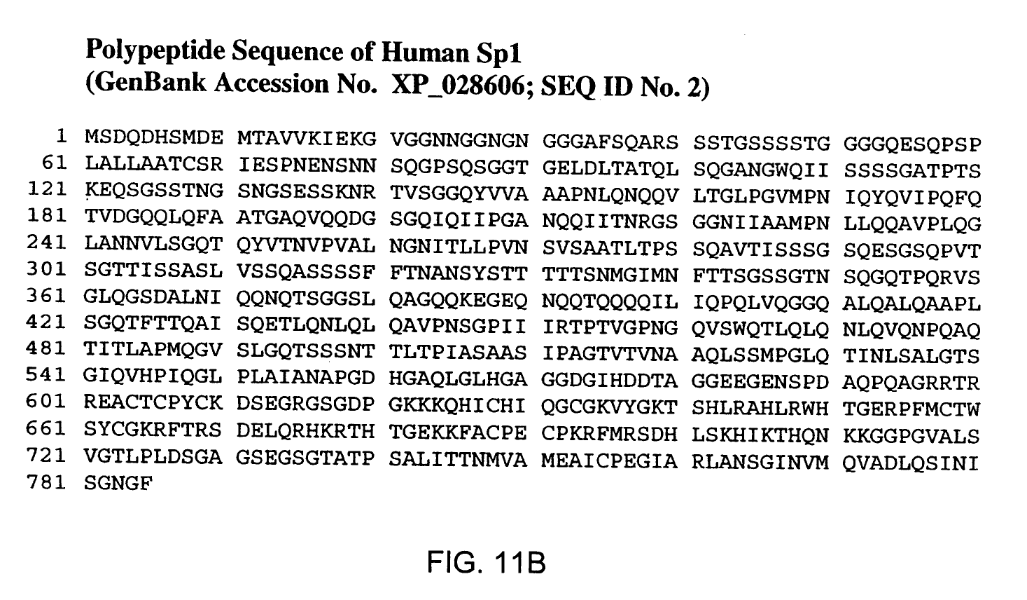

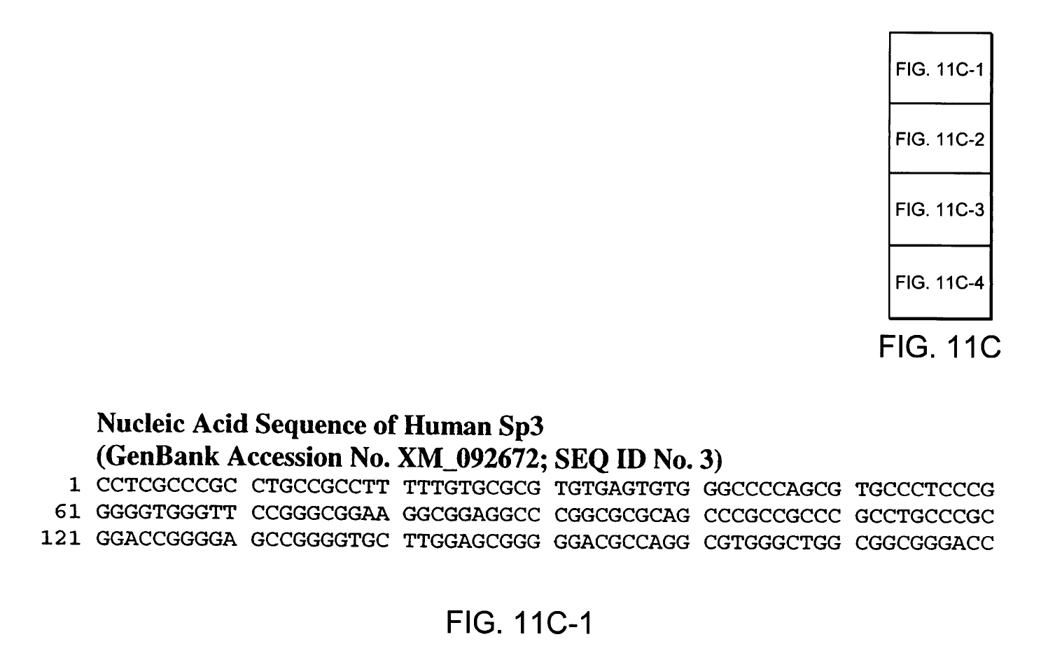

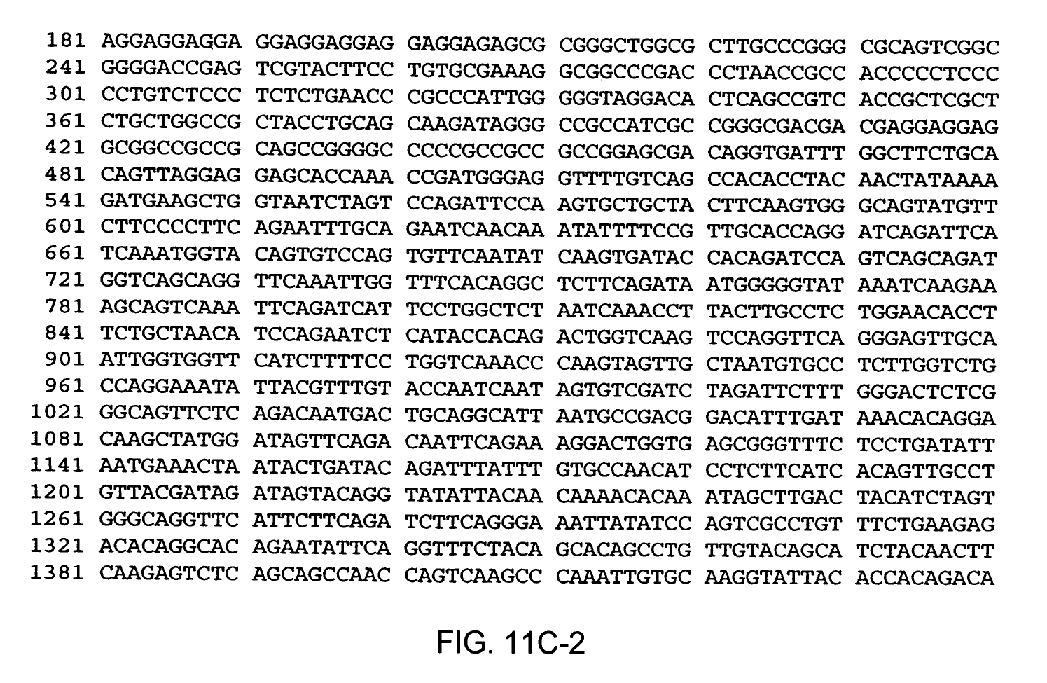

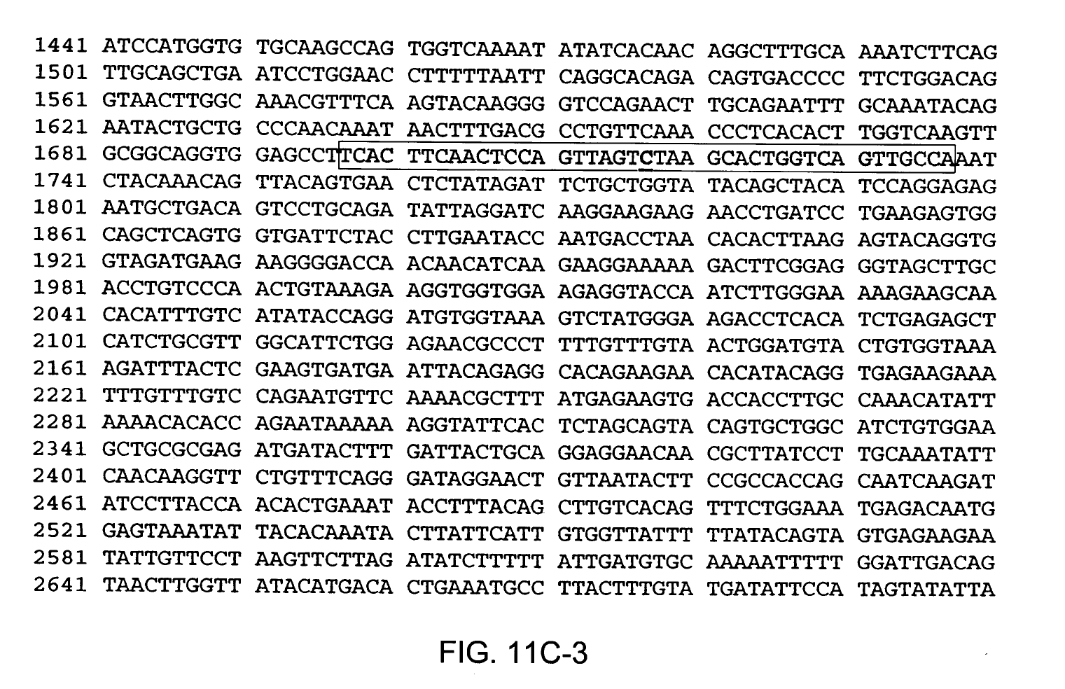

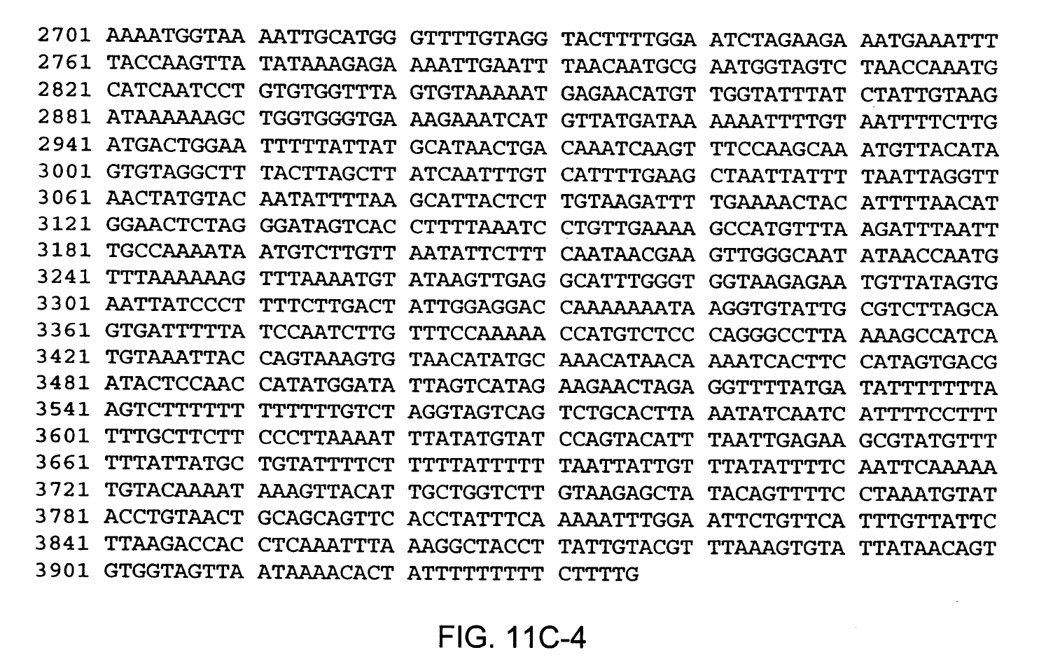

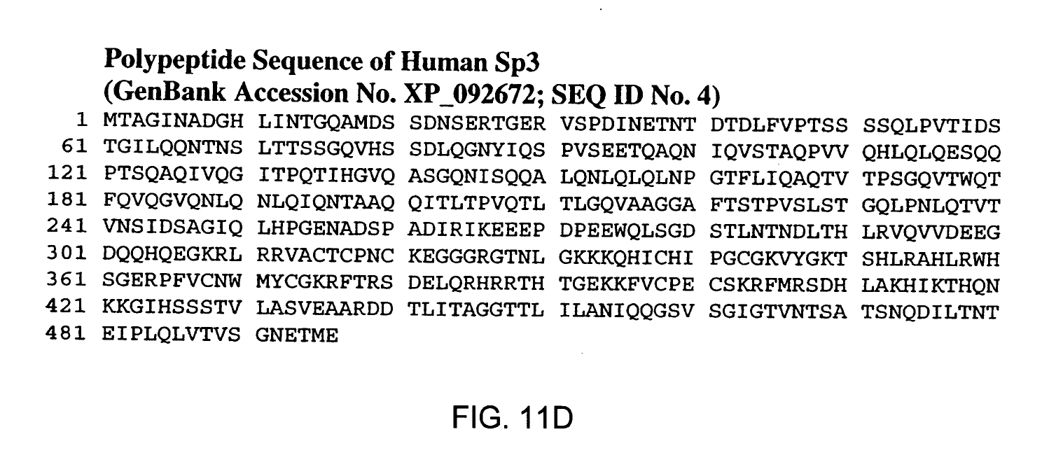

Accordingly, rAAV vectors are constructed using the strategy shown in FIG. 8. The two vectors shown at the left and right at the top of FIG. 8 are supplied by Stratagene La Jolla, Calif.). The vector shown to the upper left, designated pAAVMCS, contains the genes necessary for bacterial replication of the plasmid and ampicillin selection located between the two ITR sites (left side of vector). The vector on the upper right, designated pCMV-MCS, has two Notl sites. Between the two Noti sites is the CMV promoter, a multiple cloning site, and a poly A signal sequence (right side of vector). If the vectors shown at the top of FIG. 8 are cut with Notl, each will yield two fragments of somewhat different lengths. The pCMV fragment ends are dephosphorylated and all four fragments are gel-purified. The appropriate fragments are isolated from the gel and ligated to produce the pAAV vector shown at the bottom of FIG. 8. The Notl to Notl fragment (FIG. 8, middle right) is engineered so that it will carry one or other of the DNA sequences shown in FIG. 10 (construct A, B, or C as described below). The “a” insert carries the puromycin-resistance gene and either the Sp1 or Sp3 ribozyme, or a random sequence replacing the either or these ribozyme. For the present studies, the “X” promoter used is a constitutive promoter such as the U1snRNA promoter. This vector is particularly useful for cell culture studies because puromycin is an excellent selective agent. The “B” insert carries the gene for enhanced green fluorescent protein (EGFP) and either the Sp1 (or Sp3) ribozyme or a random DNA sequence instead of the Sp1 (or Sp3) ribozyme sequence. For the present study, the “X” is a constitutive promoter such as U1snRNA promoter. It is particularly useful for directly infecting cells to form a tumor in a mouse. The EGFP signal allows detection of cells that have been infected. The “C” insert carries the gene for puromycin-resistance and the EGFP gene. It is particularly useful as a means of determining the infectivity of the virus in cells in culture. This is determined by observing the green fluorescence of the infected cells or the number of puromycin resistant cells.

5.18. Production of rAAV Virus

The recombinant virus is produced using the Stratagene System (as described in a 31-page detailed protocol available from the company web site, [www.stratagene.com]). In brief, HEK 293 cells are used for virus production because they stably express two required adenovirus genes, E1A and E1 B. Three plasmids are co-transfected into these cells using a calcium phosphate-based protocol. The recombinant expression vectors, described above, carrying the Sp1 or Sp3 ribozyme or the EGFP, are co-transfected along with 1) the Stratagene pHelper vector which carries the adeno-virus derived genes, VA, E2A, and E4, and 2) the Stratagene pAAV-RC vector which carries the AAV-2 replication and capsid genes. The genes on the three vectors along with the E1A and E1B genes expressed by the cells supply all the trans-activating factors required for rAAV Production. Six hours following transfection, the medium is changed and the cells will be incubated for an additional 66-72 hrs. When virus production takes place, some is released into the medium and some virus is inside the cells. The medium is collected, centrifuged to remove cell debris and frozen at −80° C. as a virus stock. The cells are collected, subjected to four rounds of freezing and thawing to lyse them, and centrifuged to remove debris. The lysate is stored at −80° C. as a virus stock. Virus stocks are reported by Stratagene to contain 108 viral particles per ml and may be further concentrated, forming stocks containing up to 1012 viral particles per ml.

The titer of the resulting recombinant virus is determined, e.g., by real time quantitative PCR (RQ-PCR) for physical particle titer (see Veldwijk et al., (2002) Mol Ther. 6: 272-278. An infectious center assay (ICA) is carried out to determine the infective activity of the rAAV viruses. To measure infection activity of viruses, C12 cells from Stratagene are used because they express AAV2 rep and cap proteins (see Clark et al., (1995) Hum. Gene Ther. 6: 1329-1341). These cells are plated in 96-well plates, grown to 75% confluence, and infected with Ad5 at a MOI of 20. One microliter of serially diluted rAAV is then added to each well, and the cells are be incubated for another 42 hr. The cells are then trypsinized, suspended in PBS, and trapped on nitrocellulose by filtration under suction. The filter is probed with a radioactive probe to a viral sequence.

In order to determine the multiplicity of invention (MOI) using pancreatic cancer cells in culture, virus stocks containing the FIG. 10 “C” construct is serially diluted and added to pancreatic cancer cells in log phase growth. After a period of 6 hr, the medium is removed, the cells are washed and fresh medium is added. After 48 hr, puromycin is added at the appropriate concentration. After 2 wk, the drug resistant clones are fixed, stained, and counted. The results indicate the multiplicity of infection required to infect all the cells in the dish. Virus of this form is prepared in parallel with viruses of the FIGS. 10A or 10B form. This provides an estimate of the MOI required for infecting all of the cells in culture. The MOI used to treat tumors growing in mice is an extrapolation of this number, as understood in this technical field.

5.19. Treatment with rAAV Virus to Deplete Tumor Cell Sp1/Sp3

In order to demonstrate that the replication-defective recombinant adeno-associated virus (rAAV) constructs carrying containing the Sp1/Sp3 ribozymes are effective, the following are measured: decreases in expression of Sp1 and/or Sp3 protein; blocks in anchorage independent growth; decreases in tumor formation in athymic mice; and/or decreases in the expression of critical oncogenes carrying promoter Sp1/Sp3-binding sites.

First, the efficiency of reduction in Sp1 and Sp3 expression levels in pancreatic cancer cell lines is measured. Recombinant virus stock carrying an “A”-type (see FIG. 10A) construct is used to infect pancreatic tumor cells in culture using the amount of virus determined as above to infect all or a maximum number of cells in a population. Puromycin selection is used to identify viral-infected cells. The number of cells that die as a result of the addition of puromycin is a measure of the percent cells not infected. Drug-resistant cells are grown as a mass culture and the Sp1 and Sp3 levels determined by Western blotting.

Next, cell lines exhibiting low levels of Sp1/Sp3 are tested for their ability to form colonies in agarose and for their ability to form tumors in athymic mice. Controls are the same cells receiving the same vector, but with a random DNA sequence instead of the ribozyme. Vectors in which promoter “X” is a constitutive promoter, other such promoter such as the U1snRNA or one of the special promoters discussed below are also tested. These experiments indicate the combination of ribozymes and promoters that yield maximum reduction in expression of Sp1 and Sp3, as well as the optimal amount of virus to use to obtain maximal results. In these experiments, the virus is used as a transducing agent to infect essentially the entire population of cells. Accordingly, no selection of individual clonal populations is necessary because of the relative efficiency of gene transduction in comparison to gene transfection. These cell populations and appropriate controls are then tested for anchorage independent growth and tumor formation by s.c. injections, as described above.

Finally, it was determined whether oncogenes that are known or considered to be important in carcinogenesis in pancreatic carcinomas, and carrying promoters that contain multiple Sp1/Sp3 binding sites, are affected by the Sp1/Sp3 ribozyme constructs. Tumor cell lines in which Sp1 and/or Sp3 are markedly up-regulated show up-regulation of Sp1- and/or Sp3-controlled genes. However, since all promoters so far identified that have Sp1/Sp3 binding sites also have binding sites for other transcription factors, expression of such genes is not simply dependent upon the levels of Sp1 and Sp3 proteins. Furthermore, genes that have only a single Sp1/Sp3 site in their promoters are generally housekeeping genes and are either “on” or “off. These genes are unlikely to become oncogenes.

A number of genes that carry two or more Sp1/Sp3 sites in their promoters have been identified to be oncogenes when they are overexpressed. Such genes can be markedly up-regulated by increasing the level of Sp1 and/or Sp3, as reported above for HGF and MET expression in human fibroblastic tumor cells. In human pancreatic cancer, the VEGF, EGF-R, HGF, and cMET genes each have more than one Sp1/Sp3 site in their promoter and they act as oncogenes when they are overexpressed.

To further demonstrate that increased levels of Sp1 and/or Sp3 proteins causes the overexpression of these oncogenes, viral-infected cell lines expressing the Sp1 or Sp3 ribozyme, and which evince reduced anchorage independent growth and loss or marked reduction of tumor formation, are tested for specific expected changes in oncogene expression. Cell lines infected with the same virus carrying a random DNA sequence instead of the ribozyme serve as controls. Western blotting for the membrane proteins cMET and EGF-R and ELISA assays for the secreted proteins HGF and VEGF are performed. To determine whether one or more of these four genes, exhibiting down-regulation, is partly or wholly responsible for the tumor-forming ability of the cells, that gene is placed into an appropriate expression vector under the control of a constitutive promoter that has no Sp1/Sp3 sites and transfected into cells that are down-regulated for Sp1 expression by the Sp1 ribozyme and that can no longer form tumors. Transfectants expressing appropriate levels of the oncoprotein coded for by the oncogene in question (i.e., expressing the level seen before the Sp1/Sp3 ribozyme transfection) are then tested for the ability to make tumors and also large-size colonies in agarose. Oncogenes have a strong effect are thus identified.

In yet another measure of the effects of Sp1 and/or Sp3 ribozymes in blocking tumor formation, Affymetrix gene chip experiments are conducted to determine which genes are controlled by Sp1 and/or Sp3 and, further, which play a role in tumorigenicity. Because many genes are partially controlled by Sp1/Sp3, the genes examined are limited to the subset containing two or more Sp1/Sp3 sites in their promoters.

5.20. Subcutaneous (s.c.) Tumor Experiments

In order to demonstrate that injecting these various types of Sp1/Sp3 ribozyme-carrying recombinant viruses, which were found to be effective by the Sp1/Sp3 depletion criteria described in the preceding section, are also effective against tumors in situ, the corresponding recombinant viruses were directly injected into fluorescently-marked subcutaneous pancreatic carcinomas derived from both cell lines and directly from human pancreatic tumors as well as into primary pancreatic tumors in mice formed after transplantation of tumor fragments derived from tumors formed by the fluorescent pancreatic cell lines as a result of subcutaneous injection in other mice.

First, the recombinant Sp1/Sp3 ribozyme-carrying adeno-associated virus (rAAV) is directly injected into subcutaneous pancreatic carcinomas in mice. As noted above, pancreatic tumors growing subcutaneously in mice are the ideal place to determine whether viruses can serve as satisfactory transducing agents to express the Sp1 or Sp3 ribozymes in pancreatic cancer cells. The end point of these studies is to measure regression of the tumors, inhibition of further growth, or a reduction in the rate of growth. Direct injection of rAAV is achieved using methods known in the art (see, e.g., Ghaneh et al., (2001) Gene Ther. 8: 199-208, which demonstrates direct injection of recombinant adenovirus directly into tumors derived from subcutaneously-injected human pancreatic carcinoma cell lines in athymic mice).

Tumor size is measured weekly in order to show that the Sp1 or Sp3 ribozyme-containing viruses inhibit or slow tumor growth when compared with suitable control virus. The EGFP (viral) signal (e.g., the fluorescent label on the recombinant adenovirus) is readily monitored weekly as described above. Furthermore, experiments using the tumor cell lines labeled with DsRed protein, allow simultaneous recording of the fluorescent signal from the tumor cells (i.e., effectively treated tumor tissue marked by the red fluorescent protein and effectively infected with the green fluorescent protein recombinant adenovirus appears as yellow tissue under appropriate imaging conditions). This allows validation of the system in the pancreas of the mouse or in metastatic lesions.

The optimal size of the tumor for injection with the Sp1 or Sp3 ribozyme-carrying recombinant adeno-associated virus is determined experimentally as is the optimal MOI and the optimal frequency of injections as well as exactly how the injections are performed, etc. For example, recent studies (see Oleksiak and Crawford (2002) Mol. Biol. Evol. 19: 2026-2029), using a different virus (adenovirus) to treat human pancreatic cancer cells growing s.c. in athymic mice, provides some general information that will serve as a starting point. The tumors are allowed to develop for eight weeks, after which each tumor was injected with a total of 1011 virus particles daily over three consecutive days. The virus was injected using four needle passes, two perpendicular and two parallel to the axis of the tumor. Accordingly, this protocol is useful as a guide in optimizing the recombinant adeno-associated virus (rAAV), which is a lentivirus and not related to the adenoviruses used by Oleksiak and Crawford.

Although the autofluorescence and light scattering of the mouse make detection of the red fluorescence of the tumor cells and the green fluorescence of the virus less precise than that which can be obtained with a high-grade optimal microscope, the Lighttools' instrument described above has dual light sources with appropriate filters for separate excitation of the DsRed protein and the EGFP. This allows separate signals to be overlaid to determine whether the signals are coming from the same cells or different ones. Under optimal conditions, viruses containing the Sp1 or Sp3 ribozyme (see FIG. 10B) and the EGFP gene are used. The same construct carrying a random DNA sequence instead of the ribozyme is used as a control.

In order to optimize viral delivery, tumors are removed at various early time points and examined with a dissecting scope equipped with a suitable filters and a CCD camera to record fluorescence. Sections are cut and examined with a similarly equipped microscope. This will allows determination of the optimal methods to use to deliver viruses to obtain the highest infection frequency and the least infection of the adjacent cells. The tumor cells appear red, adjacent viral-infected cells green, and virus-infected tumor cells yellow, i.e., a combination of the red and green signals. BD-Clontech, the supplier of the DsRed gene and the EGFP gene provides examples at their web site of detection of both the DsRed and the EGFP signals from cells in culture and yellow fluorescence where both signals are present. Mah et al., ((2002) Mol. Ther. 6: 106-12) has recently demonstrated that coupling rAAV to microsphere with heparin sulfate increases the amount of virus found in cells in culture or in soft tissue of a mouse by five to ten-fold. Accordingly, this technique may be utilized in these methods to enhance viral delivery and residence within tissues.

5.21. Orthotopic Tumor Experiments

In brief, the following strategy is used to demonstrate Sp1/Sp3 ribozyme-targeted inhibition of orthotopic pancreatic tumors. Human pancreatic tissue is available from human patients. The tumor tissue is prepared as described above, and small fragments are placed subcutaneously into the right and left flank of athymic mice. Once a tumor of a suitable size (determined experimentally as described above) arises, viruses carrying the EGFP and the Sp1 or Sp3 ribozymes found to give the best effects in the other experiments are injected based on the experiment protocols developed. Whole body imaging and measuring of the tumors is carried out weekly until the tumors reach a volume of size of one cubic centimeter, at which time the animals is sacrificed, tumors are removed and fixed, and slides are prepared and studied. A necropsy of the animals is carried out to determine if there are metastases. The results obtained demonstrate that viral transduction of genes is effective in stopping or reducing in situ pancreatic tumor growth. A sample of the original tumor is lysed and Westerns blot analysis is carried out to determine the level of Sp1 and Sp3 protein in each tumor. If adjacent normal pancreatic tissue is available, it will be treated in the same manner and the levels compared.

In detail, the procedure for orthotopic transplantation of tumor fragments as described above and further detailed below is used. Subcutaneous tumors formed by pancreatic tumor-derived cells expressing the DsRed protein are used to generate the tumor fragments implanted in the pancreas. After a period of growth in the pancreas, red signal from the cancerous cells present in the pancreas is detected by whole body imaging (see e.g, Bouvet (2002) Cancer Res. 62: 1534-1540; Hoffman (2002) Lancet Oncol. 3: 546-556; Hofmann et al., (1999) Nucleic Acids Res. 27: 215-219; Bouvet et al., (2000) Clin. Exp. Metastasis 18: 213-218). When the tumor is of a suitable size as judged by the fluorescent signal, recombinant virus is injected directly into the mass of tumor cells. In a light tight room, it is possible to detect the tumor visually through a filter when the mouse is irradiated with an appropriate light source. The amount of virus, frequency of injections, etc. is as based upon results with the subcutaneous tumors as described above. The rate of growth of the tumors is determined weekly by measuring the DsRed fluorescence signal as described above. These results are important, because they demonstrate that delivery of the virus to a model “primary” tumor is effective in block or slowing growth.

5.22. Sp1 and Sp3 Ribozyme Synergy

Sp1 and Sp3 ribozyme-expressing recombinant virus (e.g., the two viruses as shown in FIG. 10A are used to co-infect pancreatic cancer cells in culture with a viruses where carries the Sp1 ribozyme and the other carries the Sp3-type ribozyme, using constructs where promoter “X” is identical and constructs where it is different. Both viruses is used at concentration known to infect only 10-25% of the cells. Puromycin selection is carried out and the surviving populations grown to large numbers and evaluated for their Sp1/Sp3 levels, tumor-forming ability, and the ability to form colonies in agarose. Control populations are infected with one or the other of the viral constructs at the same level or twice the level used when both viruses are used to infect. They are tested in the same way. This result indicates whether the effect of the two ribozymes is additive or synergistic.

5.23. Additional Viral Vectors for Delivery of Sp1/Sp3-Inhibitory and Other Therapeutic Agents

In this example additional vectors constructed for use in delivering the Sp1 or Sp3-encoding ribozymes or other Sp1/Sp3-inhibitory agents as well as other anti-oncogenic agents that may be delivered in conjunction with the Sp1/Sp3-inhibory agents of the invention are described. In addition to the viral ribozyme vector constructs shown in FIG. 10, two additional vectors were also designed. In these vectors, the EGFP gene in FIG. 10B is replaced with either the wild type p53 tumor suppressor gene or the p16INK4A gene. In each case, the gene with promoter is identical to the sequence used by Ghaneh et al., ((2001) Gene Ther. 8: 199-208). Because p53 is usually mutant and p16 is usually silenced in pancreatic carcinoma cells, these researchers used an adenovirus vector to transduce one or the other of both gene into pancreatic cancer cell and found strong growth suppression. Accordingly, viruses expressing either the p53 gene or the p16 gene linked to the Sp1 or the Sp3 ribozymes and suitable control viruses are constructed. The viruses are used to infect pancreatic tumors formed by s.c. injection of pancreatic tumor cell lines and compared for efficacy with viruses expressing the ribozyme only or the p53 only or the p16 only viruses vectors. Cells derived from such treated tumors are examined by Western blotting for expression of p53 or p16and Sp1/Sp3. The most effective viruses are additionally tested on orthotopic tumors and the animals serially sacrificed to evaluate the efficacy of the treatment. These experiments are designed so that reduction of Sp1/Sp3 levels and expression of a wild type p53 or p16 provide an additive or synergistic effect.

5.24. Promoter Optimization for Sp1 and Sp3 Ribozyme Expression

In this example, promoters are engineered for the ribozyme constructs so that they more specifically and effectively inactivate the Sp1/Sp3 mRNA and thereby increase the effectiveness of the ribozyme approach for inhibiting the growth of pancreatic tumors in patients. In particular, specific promoters are designed to act in an auto-regulatory manner to markedly reduce the high Sp1/Sp3 expression in a controlled manner in human pancreatic carcinoma-derived cell lines.

The initial studies of the effectiveness of the Sp1 and Sp3 ribozymes in human cells utilized the human U 1 snRNA promoter to drive transcription. In this example, a promoter for the Sp1 or the Sp3 ribozyme that itself contained multiple Sp1/Sp3 binding sites, so as to create a negative regulatory feedback loop, is created. In particular, in cells expressing an Sp1-regulated Sp1 ribozyme, the amount of ribozyme initially expressed is proportional to the initial level of Sp1 and/or Sp3 protein in the cells. The Sp1 ribozyme is reduce the effective steady state level of Sp1 mRNA. This results in a reduction in the steady-state level of both the Sp1 and Sp3 proteins. The experiments indicate that the Sp1 specific ribozyme regulates the levels of both Sp1 and Sp3 protein. A parallel argument can be made for the expression of the Sp3 ribozyme. The expected result is that cells that express an Sp1 or Sp3 ribozyme that had a promoter that contained only Sp1/Sp3 sites, reduce the steady state level of Sp1 and Sp3 proteins. In the human fibrosarcoma cell lines, it has been shown that tumor cells in which the level of Sp1/Sp3 was reduced to within 2 to 3 fold of normal fibroblasts no longer form tumors. Accordingly these auto-regulatory promoters are an effective way to achieve such optimal levels.

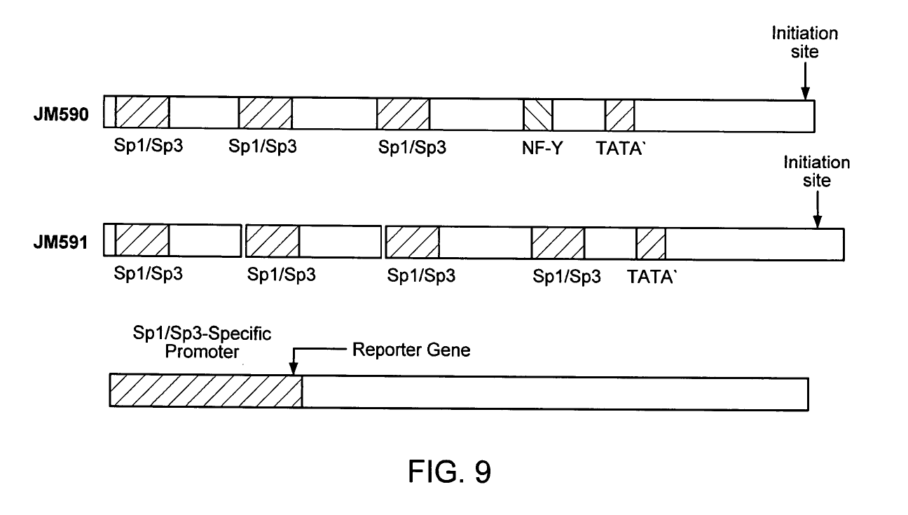

To demonstrate this negative regulatory feedback loop, two promoters designated JM590 and JM591 were designed (see FIG. 9). Promoter JM590 contains three Sp1/Sp3 sites and one NF-Y site, as well as TATA-box, and promoter JM591 contains four Sp1/Sp3 sites, as well as TATA-box. The NF-Y site was included because it is a ubiquitous transcription factor, in cells, and it has been reported that when it is present in a promoter carrying Sp1/Sp3 sites, it causes higher transcription. By means of a computer search, sequences for these promoters were chosen so that no other transcription factor will bind to them (Quandt et al. (1995) Nucleic Acids Res. 23: 4878-84). To test promoter activity, the luciferase gene was linked to the promoters and the constructs were transiently transfected into human fibroblastic tumor cells expressing high levels of Sp1 and Sp3. The original ribozyme vector (FIG. 4) was used as a positive control and the activity of the vectors with the JM promoters were determined in relation to it. Both of these promoters have been shown to be active. Furthermore, activity of these promoters may be optimized by altering, e.g., the number of nucleotides between the Sp1/Sp3 sites, the number of such sites, the distance of the NF-Y site or the Sp1/Sp3 site from the TATA box, as well as by adding additional NF-Y sites between the Sp1/Sp3 sites.

As an example of such altered Sp1/Sp3-responsive promoters, JM 590-based promoters are synthesized that have 6, 9 and 12 Sp1/Sp3 sites, JM591-based promoters that have 6 and 12 Sp1/Sp3 sites are synthesized, and JM591-based promoters are synthesized that have 8, 12, and 16 Sp1/Sp3 sites.

5.25. Sp1 and Sp3 Ribozyme Co-administration

In this example, both Sp1 and Sp3-inhibitory agents, in the form of Sp1 and Sp3 ribozymes respectively, are co-administered and the efficacy of the co-administered agents is noted. As noted in the studies above, administration of either Sp1 or Sp3 ribozymes appear to down-regulate the level of both Sp1 and Sp3 proteins in fibrosarcoma cells and in the pancreatic tumor cells that express high levels of these proteins. While not wishing to limit the invention to any single mechanistic theory, a model to account for the ability of the Sp1 ribozyme and the Sp3 ribozyme to independently down-regulate both Sp1 and Sp3 protein expression is to assume that both Sp1 and Sp3 act as transcription activators for both of these genes/proteins. Accordingly, when the Sp3 ribozyme is expressed in cells high in Sp3 protein, it cut the Sp3 mRNA and/or act as an antisense molecule for the Sp3 mRNA, which down-regulates the level of Sp3 protein. This action would results in down-regulation of Sp1 expression because there is less Sp3 protein to bind to the promoter region of the Sp1 gene. Likewise, if the Sp3 gene had functional Sp1/Sp3 binding sites in its promoter, the Sp1 ribozyme functions in a similar manner. This results in down-regulation of Sp3 protein. This model assumes that the Sp1 and Sp3 proteins each bind, not only to their own respective promoters, but also to the promoter of the other member of the pair and therefore, act in an additive or synergistic fashion in effecting Sp1/Sp3 promoter-dependent expression.

To further demonstrate this effect, the Sp3 promoter is isolated. Regulation of the Sp3 promoter by Sp1 and/or Sp3 is then confirmed using methods known in the art. This will make it possible to design promoters for, e.g., the Sp1 and Sp3 ribozymes that allow their expression to be controlled by the negative regulatory feedback loop, i.e., in cells with high levels of Sp1 or Sp3 protein, these transcription factors bind so the promoter of the ribozyme being tested, causing the cells to make higher levels of the ribozyme; cells with moderate levels of Sp1 or Sp3 will make moderate levels of the ribozyme, etc. Such a negative feedback loop system more effectively down-regulates the Sp1 and Sp3 proteins than would the same ribozyme derived by a constitutive promoter. Identification of the Sp3 promoter allows a systematic elucidation of Sp3 regulation. For example, the Sp3 minimal promoter can be determined using luciferase as a reporter gene as described below, using transient expression studies in Drosophila insect cells, i.e., cells that do not express endogenous Sp1 or Sp3, to demonstrate how the transcription factors function independently, as well as how they function when expressed together, and how expression of Sp1 and Sp3 ribozymes, driven by optimized promoters, affects expression of exogenous Sp1 and Sp3. The Sp1/Sp3-responsive negative feedback promoter construct are then used in fibrosarcoma and pancreatic carcinoma-derived cell lines, as described below.

5.26. Isolation of the Human Sp3 Promoter

To isolate the 5′ flanking region of human Sp3 gene, the human Sp3 gene sequence (AF494280) was used as bait to search the Human Genome website (http://www.ncbi.nlm.nih. gov), and found the 5′ flanking sequence of human Sp3 gene in contig AC016737. Two PCR primers with the appropriate restriction sites at each end were designed and used to isolate the 2500-nucleotide sequence of the 5′ flanking region of this gene from human placenta tissue. The DNA fragment was subcloned into pGL3-Basic luciferase reporter vector that lacks a promoter element. DNA sequencing showed that the 5′ flanking region of the human Sp3 gene was successfully obtained. Six potential Sp1/Sp3 binding sites were identified, located at positions: −1800, −700, −650, −499, −95 and −180, counting the distance from the ATG translation start codon identified by Oleksiak and Crawford (2002) Mol. Biol. Evol. 19: 2026-2029).

Although the translation start site has been located, the transcription initiation site of Sp3 gene is still unknown. To determine the transcription initiation site, RNA ligase-dependent rapid amplification of cDNA ends (5′-RLM-RACE) is carried out by using First Choice RLM-RACE kit (Ambion, Austin, Tex.). RNA is extracted from a human pancreatic tumor cell line, with high Sp1/Sp3 expression. The 5′ phosphate and the cap structure is removed from the RNA. An RNA oligonucleotide is added to the 5′ end of RNA and RT-PCR is employed to obtain the cDNAs. An adapter (specific for the RNA oligonucleotide added) and an Sp3 gene-specific primer are used to amplify the 5′ end of Sp3 transcript. The initiation site is determined by sequencing the resulting PCR product.

To determine the minimal promoter of Sp3 gene, the progressive deletion assay used by Nicolas et al., (2001) J. Biol. Chem. 276: 22126-32) is used to identify the Sp1 minimal promoter. PCR is carried out using a common reverse primer near the translational initiation site of Sp3 gene and a series of forward primers, designed to generate a series of progressively longer DNA fragments of the 5′ flanking region of the Sp3 gene. Each DNA fragment is linked to a pGL3-Basic luciferase vector that lacks a promoter element. The promoter activity for each construct when it is transiently expressed is determined from the level of luciferase activity. The shortest fragment that gives 90% or greater activity, compared with longer fragments that have the highest activity, are considered the minimal promoter. Malignant pancreatic carcinoma cell lines that have been found to coordinately express high levels of Sp1 and Sp3 protein are used for these studies (see Table 6).

Sp1/Sp3 Binding Sites present in the Sp3 minimal promoter are identified by the Matlnspector program to identify the potential Sp1/Sp3 binding sites (see Quandt et al., (1995) Nucleic Acids Res 23: 4878-84). To verify that the Sp1/Sp3 “binding” sites are active sites, mutation and EMSA assays are carried out. Each of the potential Sp1/Sp3 binding sites in the Sp3 minimal promoter is mutated individually and in all combinations using the QuikChange direct mutagenesis kit (Stratagene). These mutated Sp3 promoters are linked to the Luciferase reporter gene and co-transfected into human fibrosarcoma cell lines to examine activity. Where Sp1 and/or Sp3 proteins regulate the expression of Sp1 and Sp3 gene expression, reduced activity of the mutated reporter gene will be observed. To demonstrate the interaction between Sp1 and/or Sp3 proteins and specific Sp1/Sp3 sites in the Sp3 promoter, EMSA (Electrophoresis Mobility Shift Assay) is carried out. DNA fragments of about 20-nucleotides that have the same nucleotide sequence of each of the Sp1/Sp3 binding sites in Sp3 minimal promoter are synthesized and labeled with P32 for these studies. Mutant forms of each of these sequences are also synthesized and labeled. To carry out the EMSA experiment, nuclear extracts from the human pancreatic tumor-derived cell lines are prepared. The rest of the procedure is as described for the EMSA assays described above. Antibody to the Sp1/DNA complex or the DNA/Sp3 complex may be added to the extracts to demonstrate that specific bands contain Sp1 and which Sp3 by means of the supershift.

5.27. Sp1 and Sp3 Promoter Autoregulation by Sp1 and Sp3 Ribozymes

In order to demonstrate that Sp1 and Sp3 ribozymes effectively down-regulate Sp1 and Sp3 promoters specifically, the activity of active and disabled Sp1 and Sp3 ribozymes is compared. Disabled and mutated Sp1 and Sp3 ribozymes will not affect Sp1 and/or Sp3 promoter activity.

In order to prepare disabled Sp1 and Sp3 ribozyme constructs, a point mutation in the hammerhead ribozyme (CUGAUGA-CUAAUGA), which disables its enzymatic activity (see Tokunaga et al., (2002) Int. J. Oncol. 21: 1027-1032), is prepared. Because the antisense sequences in the ribozyme construct may act as a “traditional” antisense to inhibit translation, will synthesize the mutated Sp1 ribozyme construct with the disabled hammerhead ribozyme and mutated Sp1 antisense sequences (sequences with the same GC content but different nucleotide sequence). This mutated Sp1 ribozyme construct is subcloned into vector containing U1snRNA and the whole construct is transfected into the pancreatic cancer cells used as negative control. Parallel experiments are carried out with disabled and/or mutated Sp3 ribozyme construct.

In order to determine the enzymatic activities of the Sp1 ribozyme construct and the Sp1 disabled ribozyme, in vitro cleavage assays are carried out. The T7 promoter-Sp1 cDNA is amplified from pBSp1 FL vector by PCR using the primers 5′ TAATACGACTCACTATAG (SEQ ID NO:6) (T7 promoter)-TGGCAATAATGGGGGCAATG-3′ (SEQ ID NO:7) and 5′TGAGGTCAAGCTCACCTGT TC-3′ (SEQ ID NO:8) which amplify the Sp1 cDNA spanning the proposed cutting site (GUC) with T7 promoter. The same procedure is carried out to prepare the T7 promoter-Sp1 ribozyme cDNAs by PCR using the Sp1 ribozyme plasmids constructed above and primers specific to Sp1 ribozyme constructs (5′ TAATACGACTCACTATAG (SEQ ID NO:9) (T7 promoter), ATACTTACCTGGCAGGGGAG-3′ (SEQ ID NO:10), and 5′CAGGGGAAAGCGCGAACGCAG-3′ (SEQ ID NO:11)). The Sp1 and ribozyme RNAs are prepared by using T7 RNA polymerase (Stratagene), and Sp1 RNA is labeled with P32-dUTP. In vitro cleavage reactions are performed with these RNAs (substrate Sp1 RNA: Sp1 ribozyme RNA=1:100) at 37° C. at different Mg2+ concentrations (0-40 mM) and for a range of time (0-1 hr) and cleavage products are detected by PAGE and autoradiography. This same strategy as described above for Sp1 RNA is used to demonstrate Sp3 ribozyme enzymatic activity in vitro.

In order to demonstrate that Sp1 and Sp3 ribozymes function to block the synthesis of the Sp1 and Sp3 proteins, transient transfection of the appropriate vectors into SL2 insect followed by a luciferase assay is utilized. This system is used because: 1) the large dynamic range makes it possible to accurately determine differences; and 2) the insect cells used do not have endogenous expression of any of the Sp family members, making it possible to more clearly determine the role of the various proteins. Accordingly, vectors for these studies; the Sp1 expression vector (pPacSp1) and Sp3 expression vector (pPacUSp3) (vectors in which the insect beta-actin promoter drives Sp1 and Sp3 gene expression, Braun et al., (2001) Nucleic Acids Res. 29: 4994-5000) are utilized. Methods known in the art and as described in Suske et al., ((2000) Methods Mol. Biol. 130: 175-187) are utilized. This system allows Sp1 and Sp3 dose/response relations, and, furthermore, Sp1/Sp3 cooperativity or synergy to be readily detected. In particular, the results demonstrate whether the presence of Sp1 and Sp3 proteins have additive, synergistic or antagonistic effect on expression of genes from the Sp1/Sp3 promoter in insect cells.

In order to demonstrate that Sp1 and Sp3 ribozymes act specifically on Sp1 and Sp3 expression, the Sp1 gene is transfected using the pPacSp1 vector, the luciferase gene driven by Sp1 or the Sp3 minimal promoter and the Sp1 ribozyme or the Sp3 ribozyme driven by the U1snRNA promoters and determine the luciferase activity. Because the antisense sequence of the two ribozymes is not related, only the Sp1 ribozyme will reduce luciferase activity. Parallel experiments are carried out by transfecting the Sp3 gene using the pPacUsp3 vector and the luciferase gene driven by Sp1 or the Sp3 minimal promoters and demonstrating that co-transfection of the Sp3 ribozyme driven by the U1snRNA promoter causes reduced expression of the reporter gene. In these experiments, increased and/or decreased amounts of the ribozyme vector transfected are used to demonstrate that results specifically depend upon the amount of ribozyme.

The results demonstrate that each ribozyme inhibits activity only when the appropriate protein, e.g., Sp1 in the case of the Sp1 ribozyme and Sp3 in the case of the Sp3 ribozyme is being expressed. Disabled ribozymes are be used to confirm the specificity of the ribozymes.

5.28 Down-Regulation of Overexpressed Spil Protein in Human Fibrosarcoma Cell Lines Inhibits Tumor Formation

In this example, the role of overexpression of Sp1 was investigated by transfecting an Sp1 U1snRNA/Ribozyme into two human cell lines, malignantly transformed in culture by a carcinogen or an overexpressed oncogene, and into a patient-derived fibrosarcoma cell line. The level of expression of Sp1 in these transfected cell lines was reduced to near normal. The cells became spindle-shaped and exhibited increased apoptosis and decreased expression of several genes linked to cancer, i.e., epithelial growth factor receptor (EGFR), urokinase plasminogen activator (uPA), uPA receptor (uPAR), and vascular endothelial growth factor (VEGF). When injected into athymic mice, these cell lines with near normal levels of Sp1 failed to form tumors or did so only at a greatly reduced frequency and with a longer latency. These data indicate that overexpression of Sp1 plays a causal role in malignant transformation of human fibroblasts and suggest that for cancers in which it is overexpressed, Sp1 constitutes a target for therapy.

Materials and Methods

Cells and Cell Culture. Human fibroblast cell line MSU-1.1 has been described (Morgan, T. L., et al. (1991) Exp. Cell Res. 197: 125-136). PH2MT cells were derived from a tumor formed in athymic mice by injection of MSU-1.1 cells malignantly transformed by overexpressing H-ras (Hurlin, P. J., et al. (1989) Proc. Natl. Acad. Sci. USA 86: 187-191). γ2-3A/SB1 cells were similarly derived from a tumor formed by MSU-1.1 cells malignantly transformed by y-irradiation (O'Reilly, S., et al. (1998) Radiat. Res. 150: 577-584). SHAC cells were derived from a patient's fibrosarcoma. The cells were routinely cultured in Eagle's minimal essential medium, supplemented with L-aspartic acid (0.2 mM), L-serine (0.2 mM) and pyruvate (1 mM), (modified Eagle's medium) and 10% supplemented calf serum (Hyclone, Logan, Utah), hydrocortisone (1 μg/ml), penicillin (100 U/ml) and streptomycin (100 μg/ml) (culture medium), at 37° C. in a humidified incubator with 5% CO₂. For selection of transfected cell strains, blasticidin (10 μg/ml) was added to this culture medium.

Preparation of Sp1 Ribozyme Antisense Construct. The Sp1 U1snRNA/Ribozyme construct was constructed using the complementary oligonucleotides that encode the antisense sequence of human Sp1 (GenBank number, AJ272134) (see, e.g. Montgomery, R. A. and Dietz, H. C. (1997) Hum. Mol. Genet. 6: 519-525). The Sp1 U1snRNA/Ribozyme construct including the hammerhead ribozyme was synthesized and the double-stranded DNA was inserted between the EcoR I and Spe I sites of the pU1 vector containing the human U1snRNA and its endogenous promoter sequences. The U1snRNA/Sp1 antisense/hannmerhead ribozyme fragment was excised by BamHIl digestion and inserted into BamH I site of pCMV/Bsd vector (Invitogen, Carlsbad, Calif.). The construct was sequenced using an ABI PRISM® 3100 Genetic Analyzer (Applied Biosystems, Foster City, Calif.). The structure of the chimeric RNA (U1snRNA/Sp1 antisense/hammerhead ribozyme) was analyzed using MulFold and Loop-D-Loop programs.

Transfection. Transfection was performed using Lipofectamine (Invitogen, Carlsbad, Calif.) following the manufacturer's procedure. Transfectants were selected in medium containing 10 μg/ml blasticidin, and their Sp1 protein levels were determined by Western blot analysis.

Western Blot Analysis. Whole cell lysates were prepared using single-detergent lysis buffer as described by (Liang, et al. (2004) Int. J. Oncol. 24: 1057-1068). Conditioned-medium was prepared as described below. Protein content was quantified using the Bicinchoninic Acid Protein Assay Reagent Kit (Pierce, Rockford, Ill.), and 50 μg total protein or 20 μg conditioned-medium was loaded and separated by 7.5% sodium dodecyl sulfate-polyacrylamide gel electrophoresis (SDS-PAGE). Protein was transferred to a polyvinylidene fluoride membrane (Millipore, Billerica, Mass.), and Western blot analysis was performed using standard techniques. The signal was detected using SuperSignal reagent (Pierce, Rockford, Ill.). Antibodies against Sp1, Sp3, HGF, uPA, uPAR and EGFR were purchased from Santa Cruz (Santa Cruz, Calif.); against cMET, from Upstate (Waltham, Mass.); against Ku80, from Serotec (Raleigh, N.C.); and against β-actin, from Sigma (St. Louis, Mo.). The latter two proteins served as loading controls. Blots were quantified by densitometry. All experiments were repeated at least three times.

Preparation of Conditioned Medium. Cells were plated in culture medium at a density of 5×10⁵ per 100 mm-diameter culture dish. After 24 h, the medium was changed to serum-free medium. After another 48 h, the medium was collected and concentrated 15-20-fold using concentrators (VIVASPIN 20ML, 5,000 MWCO, Vivascience AG, Hannover, Germany).

ELISA. The level of secreted VEGF in the medium was determined using the DuoSet ELISA development system (R&D, Minneapolis, Minn.) following the manufacturer's procedures. To determine the level of VEGF in each sample, 100 μl of concentrated conditioned-medium was used. To create a standard curve, a series of two-fold serial dilutions of recombinant human VEGF (2000 ng/ml to 125 ng/ml) was included in each set of samples assayed. The concentration of VEGF in each sample was calculated by comparing the optical density of each sample to that of the standard curve and then normalizing that value to the protein concentration of each sample.

RT-PCR Analysis of Sp1 mRNA. Total RNA was extracted from logarithmically-growing cells, and 1 μg of total RNA was transcribed into cDNA using oligo dT (Schock, F., et al. (1999) Mech. Dev. 89: 125-132) (Brown, D. D., et al. (1996) Proc. Natl. Acad. Sci. USA 93: 1924-1929) (Shi, Q., et al. (2001) Cancer Res. 61: 4143-4154). Sp1 cDNA was amplified by PCR for 26 cycles with the following primers: 5′-TAATGGTGGTGGTGCCTTT-3′ and 5′-GAGATGATCTGCCAGCCATT-3′, which span the proposed hammerhead cutting site. β-actin, which served as an integrity and loading control, was amplified for 21 cycles; (β-actin primers: 5′-AGGCCAACCGCGAG AAGATGACC-3′ and 5′-GAAGTCCAGGGCGACGTAGC-3′). The PCR products were separated by 2% agarose gel, and the gel was stained with ethidium bromide.

Luciferase Assay. Cells were transiently transfected using FuGene6 (Roche, Indianapolis, Ind.), following the manufacturer's procedure. Briefly, cells in triplet were grown to 50-60% confluence in six-well plates. pRL-TK vector (Promega, Madison Wis.), 0.5 μg, was added to the cells in 1.5 μl FuGene6 transfection reagent (1:3). In parallel wells, 0.5 μg of plasmid DNA (0.01 μg pRL-CMV vector and 0.49 μg pGL2-Basic vector (Promega, Madison Wis.)) was added to cells to serve as transfection efficiency controls. The cells were incubated for 48 h, and cytosolic fractions were prepared with passive lysis buffer (Promega, Madison Wis.). The luciferase activity was analyzed using the Dual-Luciferase® Reporter Assay System (Promega, Madison Wis.) and a luminometer.

Assay for Anchorage-independence. Cells were assayed for ability to form colonies in 0.33% agarose essentially as described (Hurlin, P. J., et al. (1987) Cancer Res. 47: 5752-5757). Briefly, 5,000 cells were plated in 0.33% top agarose per 60 mm-diameter culture dish, and that layer was covered with 2 ml of culture medium. The culture medium was replaced weekly. MSU 1.1 cells were included in each assay as a negative control. After 3 wk, the cells were fixed with 2.5% glutaraldehyde, and the colonies in five randomly chosen areas of each dish were photographed using NIH Image 1.62 software. The numbers and size of the colonies in each area were calculated by Quantity One software (Bio-rad). All experiments were carried out at least three times.

Assay for Tumorigenicity. Cells were assayed for the ability to form tumors in athymic mice as described by (Liang, et al. (2004) Int. J. Oncol. 24: 1057-1068), except the mice were examined weekly for tumor growth, and the tumors were removed when they reached 1 cm in diameter. If no tumor was observed in 6 mo following injection, the mice were sacrificed.

Cell Morphology. Cells were plated in culture medium at a density of 2×10⁴ cells per well of a chamber slide and incubated at 37° C. in a humidified incubator with 5% CO₂. When the cells reached about 90% confluence, they were fixed with neutral-buffered formalin at 4° C. for 10 min. The cell morphology was observed under a Nikon eclipse TE300 microscope, and the images were recorded with a digital camera.

Cell Death Assay. Cells were plated in culture medium at 5×10⁵ cells per 100 mm-diameter culture dish and incubated at 37° C. as above. After 24 h, the medium in half the dishes was changed to serum-free medium; the cells in the other half received fresh culture medium. After 48 h of incubation, the cells floating in the medium were collected and counted, and then the attached cells were dislodged with trypsin and counted.

Apoptosis Assay. Cells were plated at a density of 2×10⁵-5×10⁵ cells per 60 mm-diameter culture dish and incubated as described. After 24 h at 37° C., the medium covering the cells was removed in order to remove any unattached cells. The attached cells were fed with fresh medium. After 24 h, the cells were detached with trypsin, stained with Anexin V-EGFP following the manufacturer's procedure (Clontech, Palo Alto, Calif.), and assayed for evidence of apoptosis using Flow Cytometry. Apoptotic cells were stained by EGFP. Non-stained cells and Fas antibody-treated cells served as negative and positive controls, respectively. The cells were stained with propidium iodide to determine whether or not the cell membrane was intact. All experiments were carried out at least three to four times.

Results

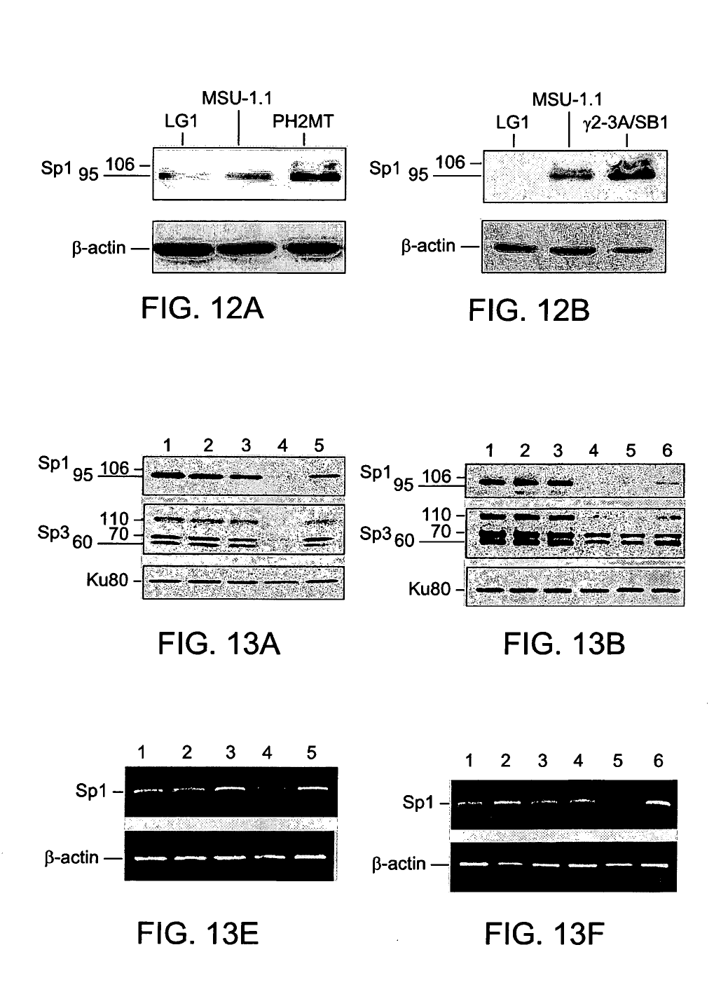

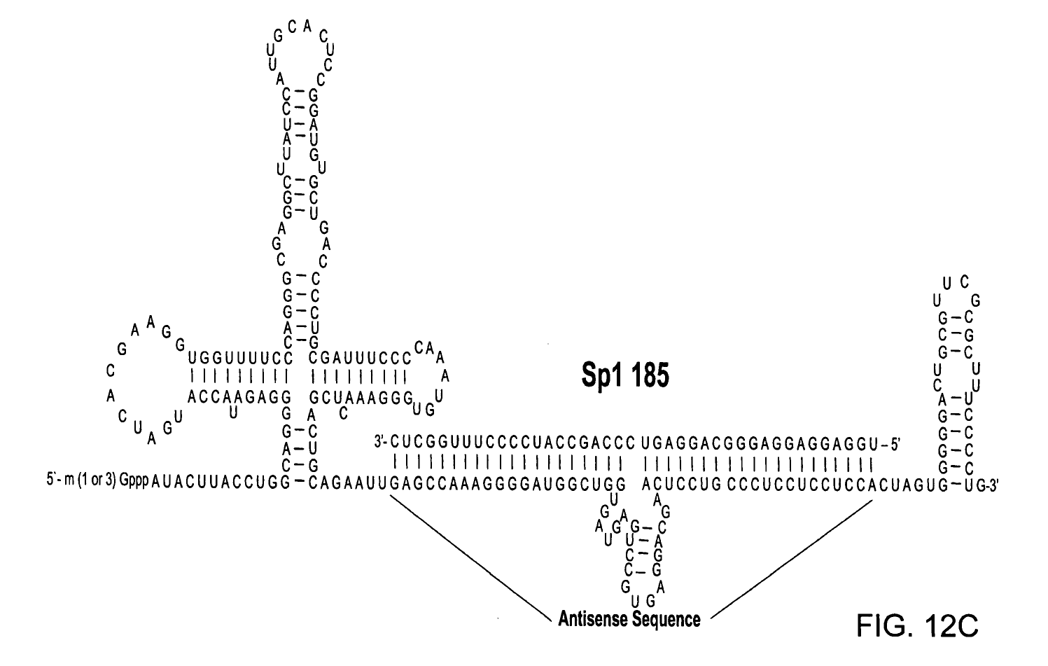

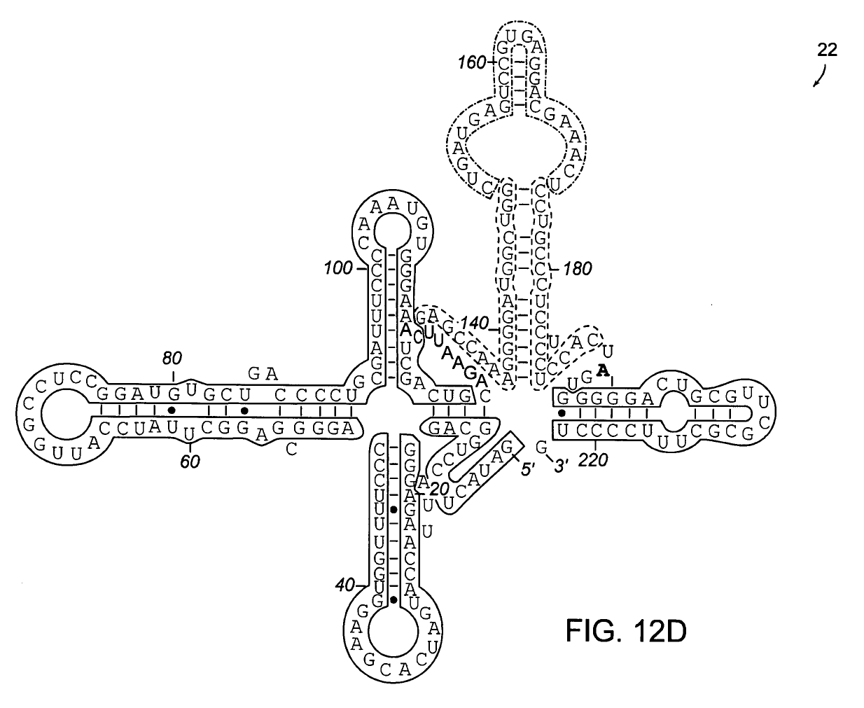

Overexpression of Sp1 in Human Fibrosarcoma Cell Lines. To confirm that the level of Sp1 is higher in human fibrosarcoma cell lines PH2MT and γ2-3A/SB1 than in their parental MSU-1.1 cells, Western blot analysis was carried out on lysates from these two cell lines, as well as from LG1, the finite life span parental cell line, from which the MSU-1.1 cells were derived (Morgan, T. L., et al. (1991) Exp. Cell. Res. 197: 125-136). The results showed that the Sp1 level was about 2-fold higher in the MSU-1.1 cells than in the LG1 cells (FIGS. 12A and 12B). The PH2MT cells had an Sp1 level 3- to 6-fold higher than the Sp1 level in the MSU-1.1 cells, and the γ2-3A/SB1 cells had an Sp1 level 7- to 10-fold higher than that of the MSU-1.1 cells.

Construction of the Sp1 U1snRNA/Ribozyme Vector. To determine whether the high expression of Sp1 observed was causally involved in the malignant transformation of these cells, an Sp1 specific ribozyme was designed and constructed to down-regulate Sp1 expression. The Sp1 U1snRNA/Ribozyme consists of three parts: an Sp1-specific antisense sequence with the hammerhead ribozyme in its center; and the two flanking regions of the U1snRNA (FIG. 12C). The Sp1-specific antisense is complementary to the 165-205 sequence of human Sp1 mRNA (Genbank accession number AJ272134). A BLAST search showed that there is no significant similarity between this sequence and that of other genes. Use of the Mulfold and Loop-D-Loop programs (Zuker, M. (1989) Science 244: 48-52) (Jaeger, J. A., et al. (1989) Proc. Natl. Acad. Sci. USA 86: 7706-7710) (Jaeger, J. A., et al. (1990) Meth. Enzymol. 183: 281-306) (FIG. 12D) to analyze the Sp1 U1snRNA/Ribozyme structure revealed that the U1snRNA structure is well conserved. To make an expression vector the Sp1 U1snRNA/Ribozyme driven by the human U1snRNA endogenous promoter was inserted into the BamH I site of the pCMV/Bsd vector containing the gene for blasticidin drug resistance.

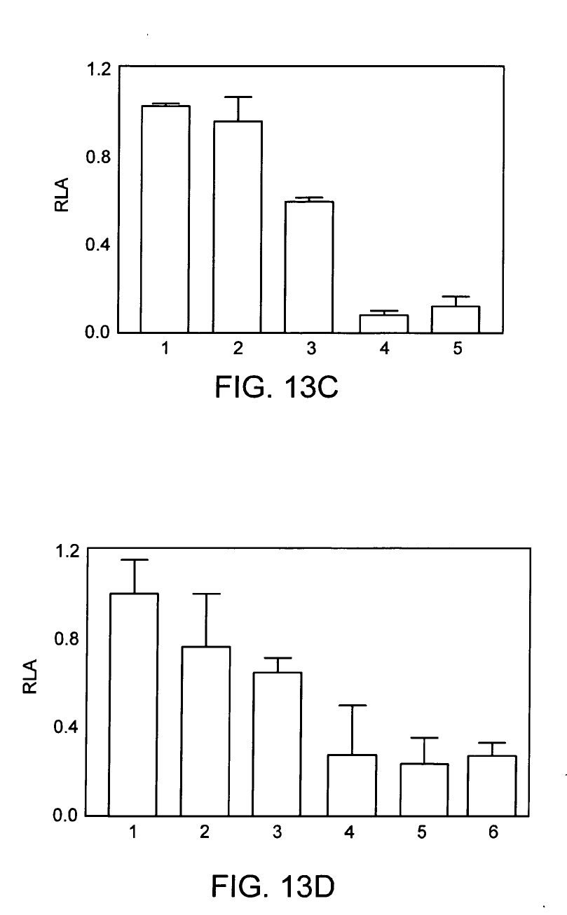

Down-regulation of Sp1 Level and Transactivating Activity. To test the ability of the Sp1 U1snRNA/Ribozyme to down-regulate Sp1 expression, PH2MT cells and γ2-3A/SB1 cells were stably transfected with the Sp1 U1snRNA/Ribozyme expression vector and drug resistance was selected for. Cells transfected with the pCMV/Bsd empty vector served as the controls. Approximately 50 drug-resistant, clonally-derived cell strains that received the Sp1 U1snRNA/Ribozyme, and 15 drug-resistant, clonally-derived cell strains transfected with the empty vector, were isolated from each cell line and screened for their Sp1 level. Two ribozyme-transfected cell strains from the PH2MT cell line (FIG. 13A, lanes 4 and 5) and three ribozyme-transfected cell strains from the γ2-3A/SB1 cell line (FIG. 13B, lanes 4-6) exhibited significantly reduced levels of Sp1 compared to their parental cells (Lane 1 of FIGS. 13A and 13B, respectively) or the empty vector-transfected cell strains (Lane 2 and 3 of FIGS. 13A and 13B, respectively). Antibodies against Sp1 and Sp3 protein were used, and a Ku80 antibody was used to detect Ku80 as the loading control.

To determine whether the Sp1 transactivating activity is reduced by down-regulation of Sp1 levels, the same cell strains as shown in FIGS. 13A and 13B were transiently transfected with a luciferase reporter construct in which the Renilla luciferase gene is driven by an HSV-TK promoter, which responds to the level of Sp1 protein. The cell lines/strains were grown to 50% confluence and transiently transfected with the HSV-TK promoter luciferase construct and the control vector. After 48 hr, whole cell lysates were prepared, and the luciferase activity was analyzed as described in the Experimental Procedures, using the Dual-Luciferase® Reporter Assay System (Promega). As shown in FIG. 13C, columns 4 and 5, and FIG. 13D, columns 4-6, compared to the parental cell line, Sp1 transactivating activity was reduced 70-90% in these Sp1 ribozyme-transfected cell strains. Two of the vector-transfected control cell strains (column 2 of FIGS. 13C and 13D) had Sp1 activity levels equal to that of their parental cells, as expected, and two had intermediate levels of Sp1 activity (column 3 of FIGS. 13C and 13D). These results demonstrate that the Sp1 U1snRNA/Ribozyme down-regulates the level of Sp1 and that this reduced level correlates with reduced transactivating activity.

Down-regulation of Sp1 Expression Reduces Expression of Sp3. Sp3 is a ubiquitously expressed transcription factor that binds to the same DNA responsive element as Sp1 and with the same affinity (Suske, G. (1999) Gene 238: 291-300). Unlike Sp1, which always acts as a transcriptional activator, Sp3 can function as an activator or a repressor of transcription, depending on secondary modifications to the Sp3 protein (Ross, S., et al. (2002) Mol. Cell. Biol. 10: 831-842) (Sapetschnig, A., et al. (2002) EMBO J. 21: 5206-5215) (Ammanamanchi, S., et al. (2003) J. Biol. Chem.). Because both Sp1 and Sp3 are ordinarily expressed in mammalian cells, the expression of genes that have the Sp1/Sp3 response elements is modulated by the combined action of Sp1 and Sp3. To determine the relative expression levels of Sp1 and Sp3, the Sp1 blots shown in FIGS. 13A and 13B were probed with an antibody specific for the human Sp3. As shown in FIG. 13A, lanes 4 and 5 and FIG. 13B, lanes 4-6, the level of Sp3 expressed correlated with the level of Sp1 expressed, and the cell strains exhibiting down-regulation of Sp1 exhibited a parallel down-regulation of Sp3. The antisense sequence of the Sp1 ribozyme was carefully examined to determine whether there was a homologous sequence in the Sp3 gene. None was found.

The Sp1 U1snRNA/Ribozyme Acts as a Ribozyme and as Antisense. To analyze the mechanisms involved in the inhibition of Sp1 expression by the Sp1 U1snRNA/Ribozyme, Sp1 mRNA levels were determined by semi-quantitative RT-PCR. Total RNA extracted from the cell strains with reduced Sp1 levels and from their parental and vector control cell strains was subjected to reverse transcription, and the levels of Sp1 mRNA were determined using a pair of Sp1-specific primers which amplify the DNA fragment spanning the proposed ribozyme cutting site (5‘GUC3′). β-actin served as the loading control. Total RNA was extracted, and the relative level of Sp1 mRNA was assayed by RT-PCR. β-actin served as the control for quantitation and determination of the integrity of the RNA. Only the cell strain used for lane 4 showed a decreased level of Sp1 mRNA. Two of the five transfectant cell strains with reduced Sp1 expression showed reduced Sp1 mRNA levels (FIG. 13E, lane 4 and FIG. 13F, lane 5) compared to the parental and vector control cell strains (lanes 1-3 of FIGS. 13E and 13F). However, the other three cell strains showed no change (FIG. 13E, lane 5 and FIG. 13F, lanes 4 and 6). These data suggest that in the former cell strains, the Sp1 U1snRNA/Ribozyme cut the Sp1 mRNA, resulting in degradation of the mRNA, whereas in the latter three cell strains, the antisense sequence inhibited translation of the Sp1 mRNA. The latter mechanism has been reported for other ribozymes (Marschall, P., et al. (1994) Cell. Mol. Neurobiol. 14: 523-538).

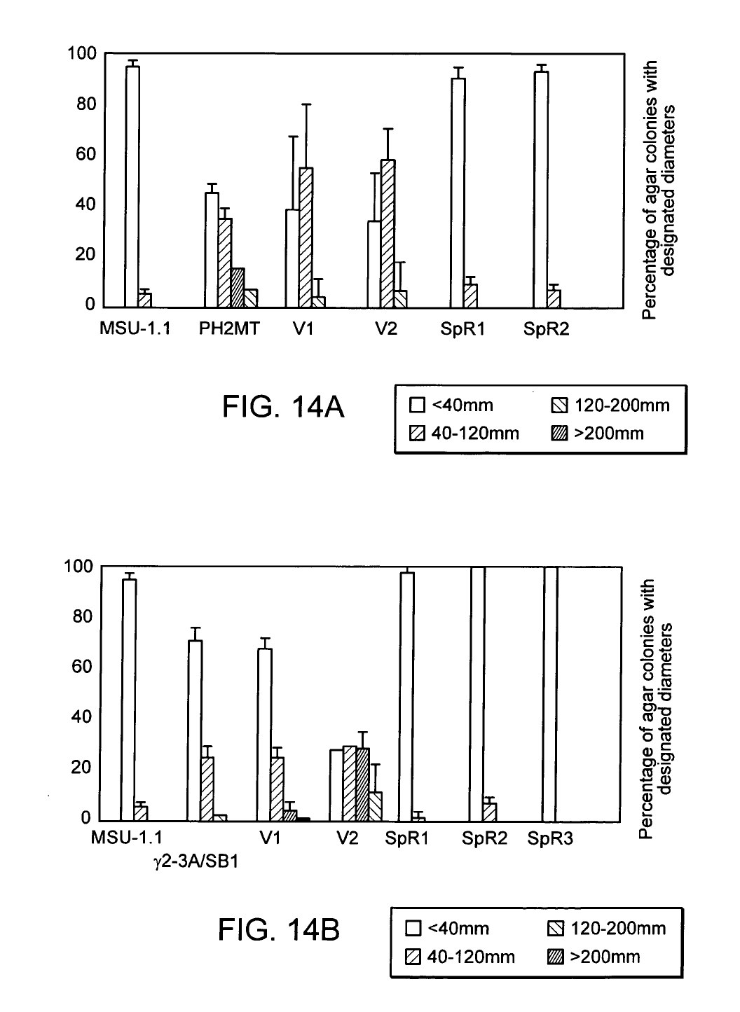

Cell Strains with Reduced Sp1 Levels No Longer Form Large Colonies in Agarose. Cell lines PH2MT and γ2-3A/SB1 are highly tumorigenic and form large-sized colonies in agarose. In contrast, their non-tumorigenic parental cell strain, MSU-1.1, forms only very small colonies (Hurlin, P. J., et al. (1989) Proc. Natl. Acad. Sci. USA 86: 187-191) (O'Reilly, S., et al. (1998) Radiat. Res. 150: 577-584). After 21 days of growth, photographs were taken of the colonies in five randomly-chosen areas of each of five dishes per group, and the average number of colonies with diameters of designated size was calculated and plotted as percent of the total. The results, shown in FIGS. 14A and 14B, demonstrate that the two ribozyme transfectants of PH2MT cells and the three from γ2-3A/SB1 cells formed very small colonies in agarose, identical to those formed by MSU-1.1 cells, whereas the vector control cell strains formed the large-sized colonies, similar to those of their parental malignant cell lines, PH2MT and γ2-3A/SB1.

Down-regulation of Sp1 Inhibits the Tumorigenicity of Cell Lines PH2MT and γ2-3A/SB1. To determine whether high expression level of Sp1 plays a role in tumor formation, athymic mice were injected with five Sp1 U1snRNA/Ribozyme transfectants showing the largest reduction in Sp1 levels (FIG. 13A, lanes 4 and 5 and FIG. 13B, lanes 4-6), as well as three ribozyme-transfected derivatives of PH2MT cells that exhibited intermediate levels of Sp1, the two parental cell strains (lane 1 of FIGS. 13A and 13B) and four vector control cell lines (lanes 2 and 3 of FIGS. 13A and 13B). Cells (10⁶) were injected s.c. into the right and left rear flank of athymic mice (two sites/mouse). Mice were examined weekly for tumors. When tumors reached ˜1 cm in diameter, they were removed. Six months after injection, these five cell strains with markedly reduced Sp1 expression (SpR1 and SpR2, derivatives of PH2MT cells and Sp1, SpR2 and SpR3, derivatives of γ2-3A/SB1 cells) had not produced any tumors (Table 7). In contrast, the parental and vector control cell strains formed large-sized tumors within 4-6 wks and three ribozyme-transfected PH2MT strains with intermediate levels of Sp1, i.e., Sp1R3, R4, and R5, produced tumors in approximately half of the sites after a longer latency.

TABLE 7
Inhibition of Tumor Formation by Down-Regulation
of Sp1 Expression in Malignant Cell Lines

| Cell | Relative Sp1 | Frequency | Latency^{b} |
|---|---|---|---|
| Lines | Level^{a} | (Tumors/Sites) | (Wk) |
| PH2MT | +++++ | 6/6 | 4 |
| V1 | +++++ | 4/4 | 4-6 |
| V2 | +++++ | 6/6 | 4-6 |
| SpR4 | ++++ | 4/6 | 5-8 |
| SpR3 | +++ | 4/4 | 8-10 |
| SpR5 | ++ | 6/16 | 12-31 |
| SpR 1 | + | 0/6c | NAd |
| SpR 2 | + | 0/6 | NA |
| γ2-A/SB1 | +++++ | 6/6 | 5-6 |
| V1 | +++++ | 5/6 | 4-5 |
| V2 | +++++ | 6/6 | 4-5 |
| SpR1 | + | 0/6 | NA |
| SpR2 | + | 0/6 | NA |
| SpR3 | + | 0/6 | NA |

^{a}Relative Sp1 level determined by Western blotting.
^{b}Time required for tumors to reach ˜1 cm.
^{c}No tumors formed within the 26-30-wk observation period.
^{d}Not applicable.
V, vector control.
SpR, transfectants expressing Sp1 U1snRNA/Ribozyme.

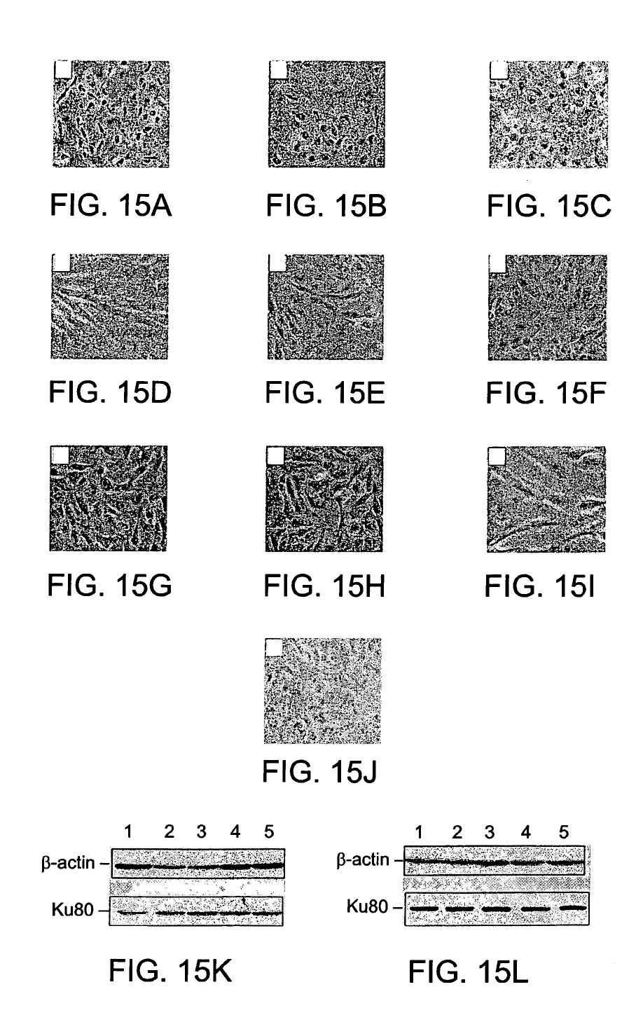

Cell Morphology Changes following the Down-regulation of Sp1 Level. As shown in FIGS. 15A-J, four cell strains with reduced Sp1 levels (D, E, I, and J) (cf. Western blotting, FIG. 13A, lanes 4 and 5; FIG. 13B, lanes 5 and 6) showed dramatic changes in morphology compared to their malignant parental strains (FIGS. 15A, 15F) and the vector transfectants (FIGS. 15B and 15C; and 15G and 15H). Cells were grown to about 90% confluence and fixed with neutral buffered formalin at 4° C. for 10 min. Photographs of the cells were taken at a magnification of 200. The bar represents 40 μm.

Western blotting assays to determine the level of expression of β-actin in the cell lines/strains described above were performed. Whole cell lysates (50 μg/lane) were analyzed using anti-β-actin antibody. Ku80 served as loading control. The cell lines shown in FIGS. 15D, 15E, and 15I had acquired a spindle-shaped morphology, similar to that of the MSU-1.1 cells from which their respective parental cell strains had been derived. The cells shown in FIGS. 15J were flatter than those shown in FIGS. 15D, 15E, and 15I, and had a round shape, intermediate between normal fibroblasts and fibrosarcoma cells. All four transfectants with reduced Sp1 level were larger than their parental cell lines and the vector control cell lines (FIGS. 15, A-C and F-H). FIGS. 15, K and L show that these changes in cell morphology in the four cell strains with reduced levels of Sp1, were not the result of changes in their level of β-actin. Such levels were constant among the parental, vector control cell strains, and ribozyme transfectants with reduced Sp1 levels.

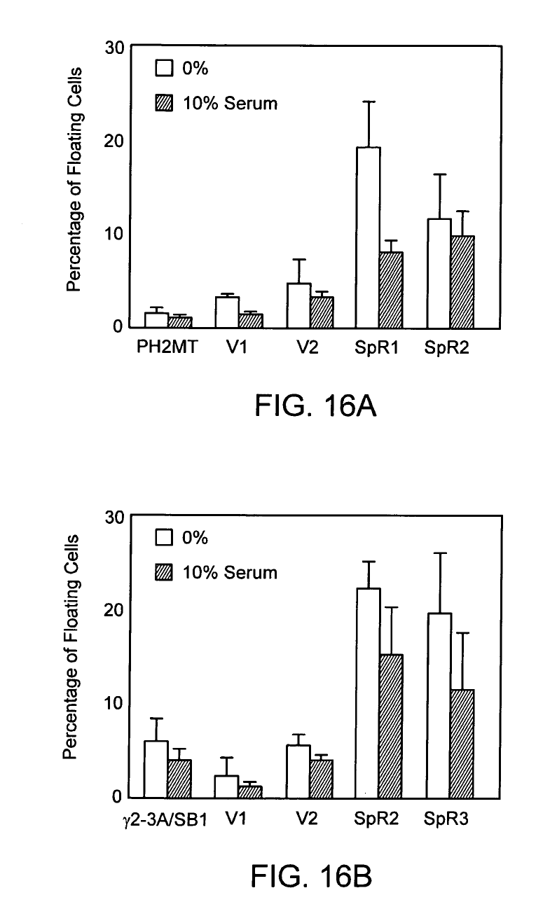

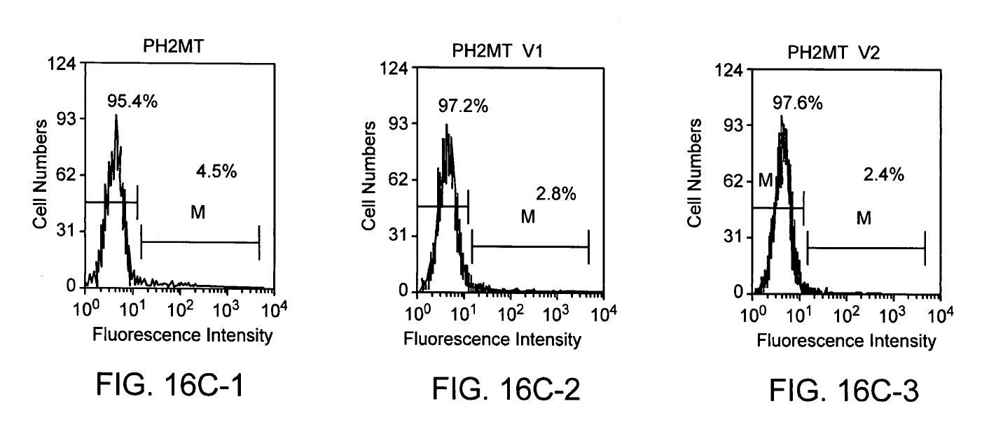

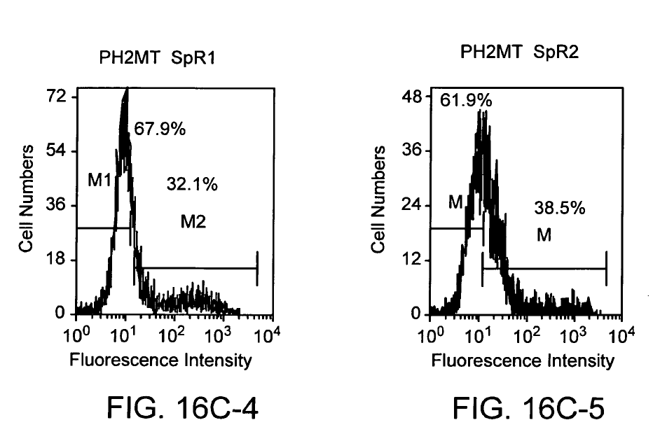

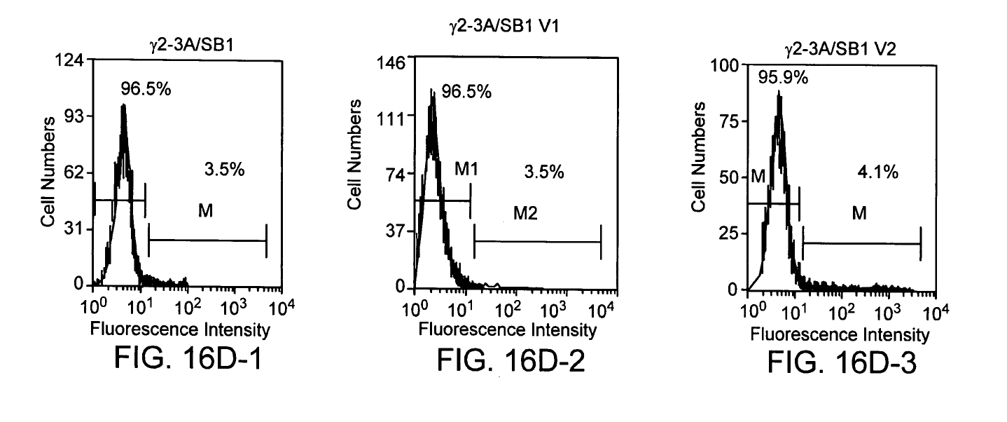

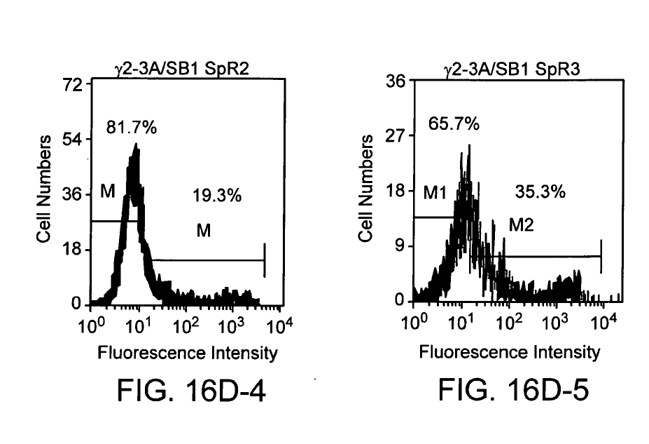

Down-regulation of Sp1 Expression Induces Apoptosis. The results shown in FIGS. 16A and 16B demonstrate that down-regulation of Sp1 expression causes cell to detach. Cells were grown in medium with or without 10% serum. The cells floating in the medium and the attached cells were collected and counted separately. The number of floating cells is shown as percentage of the total. These results demonstrate that, with or without serum in the growth medium, 8-22% of the Sp1 U1snRNA/Ribozyme transfectants of PH2MT and y2-3A/SB1 cells detached from the dish. In the parental and vector control cell strains, >8% of the cells detached from the dish. To determine whether the floating cells were dead, we collected the floating cells by centrifugation and plated them in new dishes. None of the cells attached. To determine if cell death was caused by apoptosis, the cells were grown in medium with 10% supplemented calf serum for 48 h, collected and labeled with Anexin V-EGFP and analyzed by Flow Cytometry. FIGS. 16C and 16D show the results of an apoptosis assay. The marker position shown is based on the fluorescence of unlabeled cells and cells treated with Fas antibody (100 ng/ml). The data shown are representative of 3-4 independent experiments. The ribozyme transfectants of PH2MT and γ2-3A/SB1 cells displayed a 20%-40% increase in EGFP positive cells, indicating they died by apoptosis (FIGS. 16C and 16D). The parental and vector control cells had >5% EGFP positive cells (FIGS. 16C and 16D). These results demonstrate that down-regulation of Sp1 levels correlates with increased apoptosis.

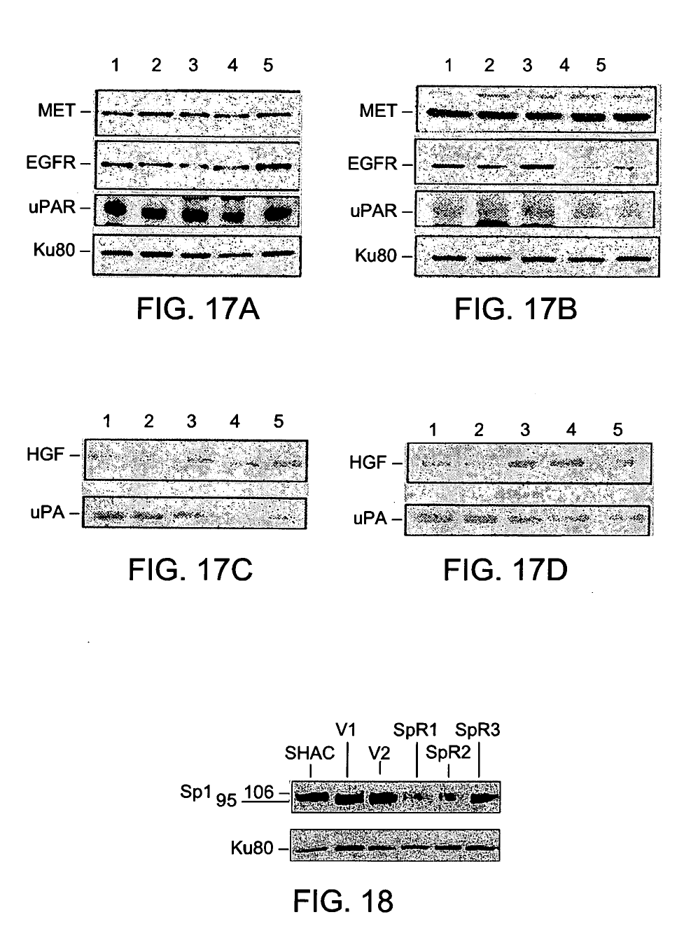

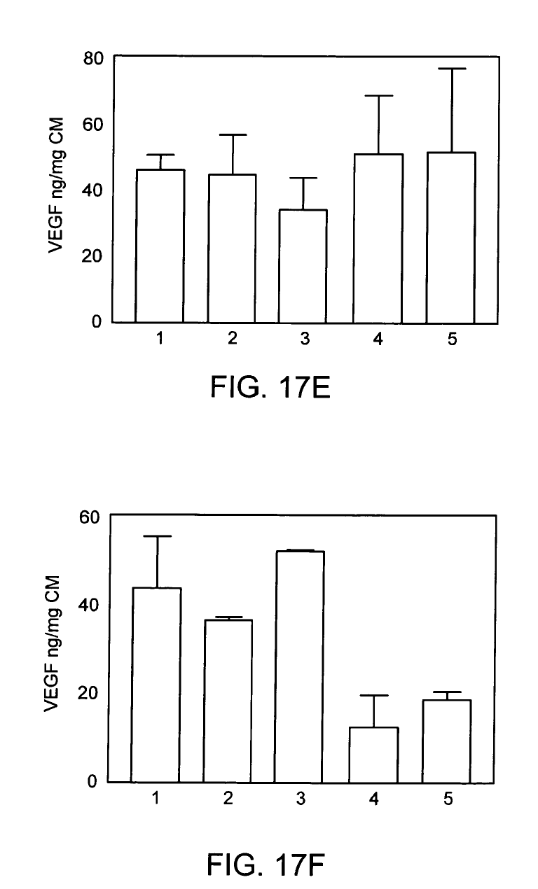

The Expression of HGF/MET, uPA/uPAR, EGFR and VEGF in the Cell Strains with Reduced Sp1 Levels. Sp1 is a transcription factor and regulates more than a thousand of genes, some of which play an important role in tumorigenesis (Black, A. R., et al. (2001) J. Cell. Physiol., 188: 143-160). To determine whether the expression level of proteins coded by Sp1-regulated genes that are thought to play a role in cancer formation is reduced in Sp1 U1snRNA/Ribozyme transfectants that exhibited down-regulated Sp1 levels, whole cell lysates were prepared and analyzed by Western blotting. Conditioned-medium from these cell strains was also prepared, concentrated, and analyzed for secreted proteins by Western blotting or ELISA. The level of the cMET, which has been shown to be higher in human fibrosarcoma cell lines (Liang, H., et al. (2004) Int. J. Oncol. 24: 1057-1068), did not change. The level of HGF, the ligand for cMET, also showed no change (FIGS. 17A-17D). However, the level of uPA, which was found to be high in 11 out of 11 human fibrosarcoma cell lines (Jankun, J., et al. (1991) Cancer Res. 51: 1221-1226), was reduced in the Sp1 U1snRNA/Ribozyme transfectants (FIGS. 17C and 17D). FIGS. 17C and 17D show expression of HGF and uPA protein in PH2MT and γ2-3A/SB1 cells, as well as vector control cell strains and Sp1-U1snRNA/Ribozyme-transfected cell strains. The cells were plated at 3×10⁵ to 5×10⁵ cells per 100-mm diameter dish in culture medium. After 24 hr, the medium was changed to serum-free medium. The conditioned-medium (CM) was collected 48 hr later, and concentrated at 4° C. The samples (20 μg) were analyzed by Western blotting with anti-HGF and anti-uPA antibodies. The levels of EGFR, VEGF and uPAR were strikingly decreased in the transfectants of γ2-3A/SB1 cell line but not in those of the PH2MT cell line (FIGS. 17A and 17B; 17E and 17F).

Down-regulation of Sp1 expression inhibits tumor formation in athymic mice by a patient-derived fibrosarcoma cell line. As the Sp1 ribozyme successfully blocked tumor formation by human fibroblasts malignantly transformed in culture, the Sp1 U1snRNA/Ribozyme construct was transfected into a patient-derived fibrosarcoma cell line (SHAC), which expresses a high level of Sp1, compared with the normal human fibroblast cell line, LG1, and tested them for tumor formation. As shown in Table 8, the two clonal populations with the largest reduction in Sp1 (see FIG. 18, Lanes 4 and 5) did not form tumors or formed tumors with a greatly increased latency and a decreased frequency, compared to the parental SHAC cells and the two vector control cell lines. Cells (10⁶) were injected s.c. into the right and left rear flank of athymic mice (two sites/mouse). Mice were examined for tumors, weekly. When tumors reached about 1 cm in diameter, they were removed. The data in parentheses are from a second experiment with the same cell lines.

TABLE 8
Inhibition of Tumor Formation by Down-regulation
of Sp1 Expression in the Malignant Cell Line, SHAC

| | Relative Sp1 | Frequency | Latency^{b} |
|---|---|---|---|
| SHAC | Level^{a} | (Tumors/Sites) | (Wks) |
| Parental | +++++ | 6/6 | 9 |
| V1 | +++++ | 3/4 | 6-8 |
| V2 | +++++ | 3/4 | 9-11 |
| SpR3 | ++ | 2/4 (3/8) | 11 (11-17) |
| SpR1 | + | 3/10 | 18-30 |
| SpR2 | + | 0/4c (1/8) | NAd (17) |

^{a}Relative Sp1 level determined by Western blotting.
^{b}Time required for tumors to reach about 1 cm.
^{c}No tumors formed within the 26-30 wk observation period.
^{d}Not applicable.
V, vector control.
SpR, transfectants expressing Sp1 U1snRNA/Ribozyme.

In FIG. 18, lane 1 shows the parental SHAC cells, lanes 2 and 3 show the empty vector transfected SHAC cells, lanes 4-6 show the U1snRNA/Ribozyme-transfected SHAC cells. The transfectant with an intermediate level of Sp1 (FIG. 18, lane 6) formed tumors with an increased latency and a decreased frequency compared to the controls. These results demonstrate that decreased expression of Sp1, caused by the Sp1 U1snRNA/Ribozyme, is effective in blocking tumor formation by a patient-derived human fibrosarcoma cell line that overexpressed Sp1.
Analysis

In these studies, Sp1 levels were successfully down-regulated by >80% without significantly affecting the doubling time of the cells in culture. The cell strains in which the level of Sp1 was near normal no longer formed tumors in athymic mice and lost the ability to form colonies in agarose. These results show a role for ‘Sp1 site’-dependent transcription in cancer. Since the Sp1 U1snRNA/Ribozyme used in these studies is specific for the Sp1 gene, these results provide direct evidence that up-regulation of Sp1 expression is involved in the malignant transformation of the fibroblasts cell lines examined. This data is consistent with the fact that when the non-tumor-forming MSU-1.1 cells are malignantly transformed, Sp1 expression is markedly increased in 65% of the cases. Those malignantly transformed cells that did not show a higher level of Sp1 became malignant by an alternative route. Furthermore, the Sp1 U1snRNA/Ribozyme transfectants of SHAC cells with reduced Sp1 levels formed no tumors in athymic mice or tumors with a greatly increased latency and decreased frequency, indicating up-regulation of Sp1 also plays a causal role in the formation of fibrosarcoma in vivo. When levels of Sp1 were reduced by >80%, the human fibrosarcoma cells reverted to a spindle cell morphology characteristic of the non-tumorigenic MSU-1.1 cells from which they were derived (Hurlin, P. J., et al. (1989) Proc. Natl. Acad. Sci. USA 86: 187-191) (O'Reilly, S., et al. (1998) Radiat. Res. 150: 577-584). Malignant transformation of cells commonly results in a morphological change (Pawlak, G. and Helfman, D. M. (2001) Curr. Opin. Genet. Dev. 11: 41-47). The data in FIG. 15 clearly indicate that the change in cell morphology in the cells with reduced Sp1 protein level is not the result of alterations of β-actin expression.

The promoters of many pro-apoptotic and anti-apoptotic genes contain “Sp1/Sp3 sites” (Rebollo, A., et al. (2000) Mol. Cell. Biol. 20: 3407-3416) (Igata, E., et al. (1999) Gene 238: 407-415) (Dong, L., et al. (1999) J. Biol. Chem. 274: 32099-32107) (Ulrich, E., et al. (1997) Genomics 44: 195-200) (Grillot, D. A., (1997) J. Immunol. 158: 4750-4757) (Ammanamanchi, S. and Brattain, M. G. (2001) J. Biol. Chem. 276: 32854-32859) (Humphries, D. E., et al. (1994) Biochem. Biophys. Res. Commun. 203: 1020-1027) (Wolf, G., et al. (2001) FEBS Lett 488: 154-159) (Yoo, J., et al. (1996) DNA Cell. Biol. 15: 377-385), which suggests that Sp1 and other members of Sp/KLF family are involved in the regulation of apoptosis. The results shown here demonstrate that down-regulation of Sp1 protein expression resulted in 20-40% of the cells undergoing apoptosis.

The HGF/SF receptor, cMET, has been demonstrated to be overexpressed in malignant human musculoskeletal rumors as well as several other types of soft tissue sarcomas (Wallenius, V., et al. (2000) Am. J. Pathol. 156: 821-829). The expression of both the HGF/SF and MET proteins showed no change in expression in the transfectants with reduced Sp1 protein levels. These results show that other transcription factors are mainly responsible for the HGF/MET promoter activity, or that a minimal level of Sp1 protein is sufficient for transcription of both genes.

The uPA protein is a key player in the regulation of cancer invasion and metastasis (Wang, Y., (2001) Med. Res. Rev. 21: 146-170). Elevated levels of uPA protein and/or mRNA have been reported in colorectal cancer (Baker, E. A. and Leaper, D. J., (2003) Eur. J. Cancer 39: 981-988), gastric cancer (Gerstein, E. S., et al. (2003) Vopr. Onkol. 49: 165-169), breast cancer (Pedersen, A. N., et al. (2003) Eur. J..Cancer 39: 899-908) (Look, M., et al. (2003) Thromb Haemost. 90: 538-548) (Battle, M. A., et al. (2003) Biochem. 42: 7270-7282), prostate cancer (Ohta, S., et al. (2003) Anticancer Res. 23: 2945-2950), head and neck adenoid cystic carcinoma (Doerr, T. D., et al. (2003) Arch. Otolaryngol. Head Neck Surg. 129: 215-218), and non small-cell lung carcinoma (Montuori, N., et al. (2003) Int. J. Cancer 105: 353-360). Inhibition of uPA activity by uPA inhibitors or down-regulation of uPA expression has been shown to suppress tumor growth in vivo and cell invasiveness in vitro (Magdolen, V., et al. (2000) Adv. Exp. Med. Biol. 477: 331-341) (Gondi, C. S., et al. (2003) Oncogene 22: 5967-5975) (Ibanez-Tallon, I., et al. (2002) Blood 100: 3325-3332). In these studies, uPA expression was shown to be significantly decreased with the down-regulation of Sp1 protein level in both cell lines examined. Taken together, these data suggest that higher uPA expression is important for the malignant transformation of human fibroblasts.

The results also demonstrate that uPAR, EGFR, and VEGF, which contribute to tumor growth and angiogenesis (Aguirre-Ghiso, J. A., et al. (2003) Cancer Res. 63: 1684-1695), display dramatic decreases in the transfectants of γ2-3A/SB1 cell line with reduced Sp1 protein, but show no change or only a slight decrease in the transfectants of the PH2MT cells with reduced levels of Sp1. These results suggest that the uPAR, EGFR and VEGF play different roles in the inhibition of the tumorigenicity of human fibrosarcoma cell lines caused by down-regulation of Sp1 expression. The γ2-3A/SB1 cells express wild type H-ras genes and the PH2MT cells express wild type p53 genes. Because both types of transformed cell lines are derived from MSU-1.1 cells, they both express the v-MYC oncogene, telomerase, and perhaps other as yet unidentified genetic changes.

Sp1 belongs to human Sp/KLF family consisting at least 21 members (Kaczynski, J., et aL (2003) Genome Biol. 4: 206). Among these members, the Sp3 shares the same expression patterns and the same binding affinity to the same DNA responsive elements as Sp1, but has different transcriptional activity (Suske, G. (1999) Gene 238: 291-300). Sp3 protein levels were high in the human fibrosarcoma cell lines with elevated levels of Sp1 protein; and low in the cells with low levels of Sp1 protein. The Sp3 protein levels decreased when the expression level of Sp1 was down-regulated by the Sp1 ribozyme antisense. The inhibition of Sp3 expression cannot be caused directly by the Sp1 U1snRNA/Ribozyme because there is no similarity between the sequences of the Sp1 U1snRNA/Ribozyme sequence and the Sp3 cDNA. Therefore, the Sp1 is acting as a transcription factor regulates the transcription of the Sp3 gene, and the down-regulation of the Sp1 protein level reduces the level of Sp3 gene transcription.

Surprisingly, the Sp1 protein, when functioning as an oncoprotein, exhibits specificity in up-regulation of other genes (e.g. oncogenes) although it controls more than a thousand genes (Suske, G. (1999) Gene 238: 291-300). Sp1 is ubiquitously expressed and regulates the expression of genes that have a single Sp1 site in their promoters as well as genes that have multiple Sp1 sites in their promoters. The studies presented here provide direct evidence that up-regulation of Sp1 expression plays a causal role in the malignant transformation of human fibroblasts. Given the important role of Sp1 in the regulation of cell growth, invasiveness/metastasis, angiogenesis and cell apoptosis, these data show that down-regulation of Sp1 protein levels or inhibition of its transactivating activity in cancer cells in which Sp1 is over expressed is a useful therapeutic strategy.

Equivalents and Incorporation by Reference

Those skilled in the art will recognize, or be able to ascertain using no more than routine experimentation, many equivalents of the specific embodiments of the invention described herein. Such equivalents are intended to be encompassed by the following claims.

The contents of all cited references including literature references, issued patents, published or non published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature (see, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait, ed., 1984); Mullis et al., U.S. Pat. No. 4,683,195; Nucleic Acid Hybridization (B. D. Hames & S. J. Higgins eds. 1984); Transcription and Translation (B. D. Hames & S. J. Higgins eds. 1984); (R. 1. Freshney, Alan R. Liss, Inc., 1987); Immobilized Cells and Enzymes (IRL Press, 1986); B. Perbal, A Practical Guide to Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al., eds.) Immunochemical Methods in Cell and Molecular Biology (Mayer and Walker, eds., Academic Press, London, 1987); Handbook of Experimental Immunology, Volumes I-IV (D. M. Weir and C. C. Blackwell, eds., 1986) (Cold Spring Harbor Laboratory Press, Cold Spring Harbor, N.Y., 1986).

1. A method of inhibiting the growth of an Sp1/Sp3-expressing cancerous cell, comprising contacting the cell with an Sp1/3-inhibitory agent so as to decrease the level of Sp1/Sp3, thereby inhibiting the growth of the Sp1/Sp3-expressing cancerous cell.

2. The method of claim 1, wherein the Sp1/Sp3-inhibitory agent is selected from the group consisting of an Sp1-targeted ribozyme, an Sp1-targeted antisense oligonucleotide, and an Sp1-targeted siRNA.

3. The method of claim 1, wherein the Sp1/Sp3-inhibitory agent is an Sp1-targeting ribozyme.

4. The method of claim 3, wherein the Sp1-targeting ribozyme has the sequence shown in FIG. 4A.

5. The method of claim 1, wherein the Sp1/Sp3-inhibitory agent is selected from the group consisting of an Sp3-targeted ribozyme, an Sp3-targeted antisense oligonucleotide, and an Sp3-targeted siRNA.

6. The method of claim 1, wherein the Sp1/Sp3-inhibitory agent is selected from the group consisting of an Sp1 transcription factor decoy oligonucleotide, an Sp1 site triplex forming oligonucleotide, and an Sp1-binding aptamer.

7. The method of claim 1, wherein the Sp1/Sp3- inhibitory agent is selected from the group consisting of an Sp3 transcription factor decoy oligonucleotide, an Sp3 site triplex-forming oligonucleotide, an Sp3-binding aptamer.

8. The method of claim 1, wherein the Sp1/Sp3-inhibitory agent is selected from the group consisting of an Sp1targeting proteolytic agent, an Sp1 dominant negative molecule, and an Sp1 binding small molecule.

9. The method of claim 1, wherein the Sp1/Sp3-inhibitory agent is selected from the group consisting of an Sp3-targeting proteolytic agent, an Sp3 dominant negative molecule, and an Sp3-binding small molecule.

10. The method of claim 1, wherein the cancerous cell is a fibrosarcoma.

11. The method of claim 1, wherein the cancerous cell is selected from the group consisting of a pancreatic cell, a fibroblast, a breast cell, a gastric cell, and a thyroid cell.

12. The method of claim 1, wherein the Sp1/Sp3-inhibitory agent is expressed from an Sp1/Sp3-autoregulatory expression system.

13. The method of claim 12, wherein the Sp1/Sp3-autoregulatory expression system comprises an Sp1 gene promoter sequence or an Sp1/Sp3 binding site.

14. A method of treating or preventing a cancerous growth or condition associated with Sp1/Sp3 overexpression in a subject, comprising administering an Sp1/Sp3-inhibitory agent to the subject so as to decrease the level of Sp1/Sp3 overexpression, thereby treating or preventing the cancerous growth or condition associated with Sp1/Sp3 overexpression in the subject.

15. The method of claim 14, wherein the Sp1/Sp3-inhibitory agent is selected from the group consisting of an Sp1-targeted ribozyme, an Sp3-targeted ribozyme, an Sp1-targeted antisense oligonucleotide, an Sp3-targeted antisense oligonucleotide, an Sp1-targeted siRNA, and an Sp3-targeted siRNA.

16. The method of claim 14, wherein the Sp1/Sp3-inhibitory agent is an Sp1-targeting ribozyme.

17. The method of claim 16, wherein the Sp1-targeting ribozyme has the sequence shown in FIG. 4A.

18. The method of claim 14, wherein the Sp1/Sp3-inhibitory agent is selected from the group consisting of an Sp-targeting proteolytic agent, an Sp3-targeting proteolytic agent, an Sp1 dominant negative molecule, an Sp3 dominant negative molecule, an Sp1-binding aptamer, an Sp3-binding aptamer, an Sp1-binding small molecule, an Sp3-binding small molecule, an Sp1/Sp3 transcription factor decoy oligonucleotide, and an Sp1/Sp3 site triplex-forming oligonucleotide.

19. The method of claim 14, wherein the cancerous growth or condition is a fibrosarcoma.

20. The method of claim 14, wherein the cancerous growth or condition is a cancer selected from the group consisting of pancreatic cancer, fibroblast cancer, breast cancer, gastric cancer, and thyroid cancer.

21. The method of any of claim 14-20, wherein the Sp1/Sp3-inhibitory agent is expressed from an Sp1/Sp3-autoregulatory expression system.

22. The method of claim 21, wherein the Sp1/Sp3-autoregulatory expression system comprises an Sp1 gene promoter sequence or an Sp1/Sp3 binding site.

23. A nucleic acid for expression of a tumor cell recombinant therapeutic agent in an Sp1/Sp3 responsive-manner, comprising an Sp1/Sp3-responsive expression system functionally linked to a sequence encoding the tumor cell recombinant therapeutic agent.

24. The nucleic acid of claim 23, wherein the tumor cell therapeutic agent is an Sp1/Sp3-inhibitory agent.

25. The nucleic acid of claim 24, wherein the Sp1/Sp3-inhibitory agent is expressed from the Sp1 /Sp3-responsive expression system in an auto-regulatory manner.

26. The nucleic acid of claim 24, wherein the Sp1/Sp3-inhibitory agent is selected from the group consisting of an Sp1-targeted ribozyme, an Sp3-targeted ribozyme, an Sp1-targeted antisense oligonucleotide, an Sp3-targeted antisense oligonucleotide, an Sp1-targeted siRNA, and an Sp3-targeted siRNA.

27. The nucleic acid of claim 24, wherein the Sp1/Sp3 inhibitory agent is an Sp1-targeting ribozyme.

28. The nucleic acid of claim 27, wherein the Sp1-targeting ribozyme has the sequence shown in FIG. 4A.

29. The nucleic acid of claim 24, wherein the Sp1/Sp3-inhibitory agent is selected from the group consisting of an Sp1 targeting proteolytic agent, an Sp3-targeting proteolytic agent an Sp1-targeted dominant negative agent and an Sp3-targeted dominant negative agent.

30. The nucleic acid of claim 23, wherein the Sp1/Sp3-responsive expression system comprises an Sp1 gene promoter sequence.

31. The nucleic acid of claim 23, wherein the Sp1/Sp3-responsive expression system comprises a plurality of Sp1/Sp3-binding sites.

32. The nucleic acid of claim 31, wherein the Sp1/Sp3-responsive expression system comprises at least two Sp1/Sp3-binding sites sequences selected from the group consisting of GGGCGG and GGGGCGGGG.

33. The nucleic acid of any of claims 23-32, further comprising a vector nucleic acid sequence.

34. The nucleic acid of claim 33, wherein the vector nucleic acid sequence is an adeno-associated vector sequence.

35. A method of detecting the treatment of tumor cells in situ comprising:
providing a mammalian organism expressing a first fluorescent protein from a tumor cell;
administering to the mammalian organism a nucleic acid that encodes a tumor cell therapeutic agent and expresses a second fluorescent protein; and
detecting the co-expression of the first and the second fluorescent proteins, wherein co-expression of the first and the second fluorescent proteins indicates that the tumor cell therapeutic agent has treated the tumor cells.

36. The method of claim 35, wherein the tumor cell is a fibrosarcoma.

37. The method of claim 36, wherein the tumor cell is selected from the group consisting of a pancreatic cell, a fibroblast cell, a breast cell, a gastric cell, and a thyroid cell.

38. The method of claim 35 wherein the fluorescent proteins are green fluorescent protein and red fluorescent protein, and co-expression is detected as yellow fluorescence.

39. A method of detecting in situ the treatment of a tumor or cancerous growth associated with Sp1/Sp3 overexpression in a mammalian organism, comprising:
providing a mammalian organism expressing a first fluorescent protein in a cancerous cell overexpressing Sp1/Sp3;
administering to the mammalian organism a nucleic acid that encodes an Sp1/Sp3-inhibitory agent and expresses a second fluorescent protein; and
detecting the co-expression of the first and the second fluorescent proteins, wherein co-expression of the first and the second fluorescent protein indicates treatment of the cacnerous cells by the Sp1/Sp3-inhibitory agent.

40. The method of claim 39, wherein the fluorescent proteins are green fluorescent protein and red fluorescent protein, and expression is detected as yellow fluorescence.

41. The method of claim 39, wherein the Sp1/Sp3-inhibitory agent is selected from the group consisting of an Sp1 targeted ribozyme, an Sp targeted ribozyme, an Sp1-targeted antisense oligonucleotide, an Sp3-targeted antisense oligonucleotide, an Sp1-targeted siRNA, and an Sp3-targeted siRNA.

42. The method of claim 39, wherein the Sp1/Sp3-inhibitory agent is an Sp1-targeting ribozyme.

43. The method of claim 42, wherein the Sp1-targeting ribozyme has the sequence shown in FIG. 4A.

44. The method of claim 39, wherein the Sp1/Sp3-inhibitory agent is selected from the group consisting of an Sp1-targeting proteolytic agent, and an Sp3-targeting proteolytic agent.

45. The method of claim 39, wherein the Sp1/Sp3-inhibitory agent is selected from the group consisting of an Sp1/Sp3-binding site oligonucleotide, an Sp1/Sp3-competitive binding agent, and mitomycin C.

46. The method of claim 39, wherein the Sp1/Sp3-inhibitory agent is expressed from an autoregulatory Sp1/Sp3-responsive expression system.

47. The method of claim 39, wherein the tumor or cancerous growth associated with Sp1/Sp3 overexpression is a fibrosarcoma.

48. The method of claim 39, wherein the tumor or cancerous growth associated with Sp1/Sp3 overexpression is selected from the group consisting of pancreatic cancer, fibroblast cancer, breast cancer, gastric cancer, and thyroid cancer.

49. A method of treating a subject suffering from a pancreatic or fibroblast cancerous growth associated with Sp1/Sp3-overexpression, comprising administering to the subject an Sp1-inhibitory agent or an Sp3-inhibitory agent so as to decrease the level of Sp1 and/or Sp3, thereby inhibiting the growth of the Sp1/Sp3-overexpressing cancerous cell,
wherein the Sp1/Sp3-inhibitory agent is an Sp1-targeted ribozyme or an Sp3-targeted ribozyme, and
wherein said Sp1-targeted ribozyme or Sp3-targeted ribozyme is expressed in an Sp1/Sp3-responsive manner from an Sp1/Sp3-responsive expression system that includes an an Sp1 gene promoter sequence or a plurality of G/C box Sp1/Sp3-binding sites.

50. The method of claim 49, wherein the pancreatic or fibroblast cancerous growth associated with Sp1/Sp3-overexpression is treated by administering an effective amount of an Sp1-targeting ribozyme having the sequence shown in FIG. 4A.


SEQUENCE LISTING (22 sequences)

SEQ ID NO: 1 - DNA, 3024 bp, Homo sapiens
gtccgggttcgcttgcctcgtcagcgtccgcgtttttcccggccccccccaacccccccggacaggacccccttgagcttgtccctcagctgccaccatgagcgaccaagatcactccatggatgaaatgacagctgtggtgaaaattgaaaaaggagttggtggcaataatgggggcaatggtaatggtggtggtgccttttcacaggctcgaagtagcagcacaggcagtagcagcagcactggaggaggagggcaggagtcccagccatcccctttggctctgctggcagcaacttgcagcagaattgagtcacccaatgagaacagcaacaactcccagggcccgagtcagtcagggggaacaggtgagcttgacctcacagccacacaactttcacagggtgccaatggctggcagatcatctcttcctcctctggggctacccctacctcaaaggaacagagtggcagcagtaccaatggcagcaatggcagtgagtcttccaagaatcgcacagtctctggtgggcagtatgttgtggctgccgctcccaacttacagaaccagcaagttctgacaggactacctggagtgatgcctaatattcagtatcaagtaatcccacagttccagaccgttgatgggcaacagctgcagtttgctgccactggggcccaagtgcagcaggatggttctggtcaaatacagatcataccaggtgcaaaccaacagattatcacaaatcgaggaagtggaggcaacatcattgctgctatgccaaacctactccagcaggctgtccccctccaaggcctggctaataatgtactctcaggacagactcagtatgtgaccaatgtaccagtggccctgaatgggaacatcaccttgctacctgtcaacagcgtttctgcagctaccttgactcccagctctcaggcagtcacgatcagcagctctgggtcccaggagagtggctcacagcctgtcacctcagggactaccatcagttctgccagcttggtatcatcacaagccagttccagctcctttttcaccaatgccaatagctactcaactactactaccaccagcaacatgggaattatgaactttactaccagtggatcatcagggaccaactctcaaggccagacaccccagagggtcagtgggctacaggggtctgatgctctgaacatccagcaaaaccagacatctggaggctcattgcaagcaggccagcaaaaagaaggagagcaaaaccagcagacacagcagcaacaaattcttatccagcctcagctagttcaagggggacaggccctccaggccctccaagcagcaccattgtcagggcagacctttacaactcaagccatctcccaggaaaccctccagaacctccagcttcaggctgttccaaactctggtcccatcatcatccggacaccaacagtggggcccaatggacaggtcagttggcagactctacagctgcagaacctccaagttcagaacccacaagcccaaacaatcaccttagccccaatgcagggtgtttccttggggcagaccagcagcagcaacaccactctcacacccattgcctcagctgcttccattcctgctggcacagtcactgtgaatgctgctcaactctcctccatgccaggcctccagaccattaacctcagtgcattgggtacttcaggaatccaggtgcacccaattcaaggcctgccgttggctatagcaaatgccccaggtgatcatggagctcagcttggtctccatggggctggtggtgatggaatacatgatgacacagcaggtggagaggaaggagaaaacagcccagatgcccaaccccaagccggtcggaggacccggcgggaagcatgcacctgcccctactgtaaagacagtgaaggaaggggctcgggggatcctggcaaaaagaaacagcatatttgccacatccaaggctgtgggaaagtgtatggcaagacctctcacctgcgggcacacttgcgctggcatacaggcgagaggccatttatgtgtacctggtcatactgtgggaaacgcttcacacgttcggatgagctacagaggcacaaacgtacacacacaggtgagaagaaatttgcctgccctgagtgtcctaagcgcttcatgaggagtgaccacctgtcaaaacatatcaagacccaccagaataagaagggaggcccaggtgtagctctgagtgtgggcactttgcccctggacagtggggcaggttcagaaggcagtggcactgccactccttcagcccttattaccaccaatatggtagccatggaggccatctgtccagagggcattgcccgtcttgccaacagtggcatcaacgtcatgcaggtggcagatctgcagtccattaatatcagtggcaatggcttctgagatcaggcacccggggccagagacatatgggccataccccttaaccccgggatgcaaggtagcatgggtccaagagacatggaagagagagccatgaagcattaaaatgcatggtgttgagaagaatcaggagagggatacaagagaggagatggggtcccggcacccatctgtatcatcagtgcctctttgaaggtgggaaacattagtgaaaattctgttggtgccacgctttgatgagcatttgtttgaccccagtttcttcttacacttcttaccccagcctacccttcctgcatttctcttctcagctcttccatgatggattcccccccctttcctaaagccatcatgccttgataaatatatatgatcattgaaatactttttaacaaaaaacagattctatattattatatatatatatatatatataaagatatatagagatgcattcacaggggttggctgggaggaggaagaccattctgtgaccaaaataccttggtcattttttttatattgccttatttccctatggctgagccttgttgtgacacatcaagctt

SEQ ID NO: 2 - protein, 785 aa, Homo sapiens
MSDQDHSMDEMTAVVKIEKGVGGNNGGNGNGGGAFSQARSSSTGSSSSTGGGGQESQPSPLALLAATCSRIESPNENSNNSQGPSQSGGTGELDLTATQLSQGANGWQIISSSSGATPTSKEQSGSSTNGSNGSESSKNRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQTQYVTNVPVALNGNITLLPVNSVSAATLTPSSQAVTISSSGSQESGSQPVTSGTTISSASLVSSQASSSSFFTNANSYSTTTTTSNMGIMNFTTSGSSGTNSQGQTPQRVSGLQGSDALNIQQNQTSGGSLQAGQQKEGEQNQQTQQQQILIQPQLVQGGQALQALQAAPLSGQTFTTQAISQETLQNLQLQAVPNSGPIIIRTPTVGPNGQVSWQTLQLQNLQVQNPQAQTITLAPMQGVSLGQTSSSNTTLTPIASAASIPAGTVTVNAAQLSSMPGLQTINLSALGTSGIQVHPIQGLPLAIANAPGDHGAQLGLHGAGGDGIHDDTAGGEEGENSPDAQPQAGRRTRREACTCPYCKDSEGRGSGDPGKKKQHICHIQGCGKVYGKTSHLRAHLRWHTGERPFMCTWSYCGKRFTRSDELQRHKRTHTGEKKFACPECPKRFMRSDHLSKHIKTHQNKKGGPGVALSVGTLPLDSGAGSEGSGTATPSALITTNMVAMEAICPEGIARLANSGINVMQVADLQSINISGNGF

SEQ ID NO: 3 - DNA, 3936 bp, Homo sapiens
cctcgcccgcctgccgcctttttgtgcgcgtgtgagtgtgggccccagcgtgccctcccgggggtgggttccgggcggaaggcggaggcccggcgcgcagcccgccgcccgcctgcccgcggaccggggagccggggtgcttggagcgggggacgccaggcgtgggctggcggcgggaccaggaggaggaggaggaggaggaggagagcgcgggctggcgcttgcccgggcgcagtcggcggggaccgagtcgtacttcctgtgcgaaaggcggcccgaccctaaccgccaccccctccccctgtctccctctctgaacccgcccattgggggtaggacactcagccgtcaccgctcgctctgctggccgctacctgcagcaagatagggccgccatcgccgggcgacgacgaggaggaggcggccgccgcagccggggcccccgccgccgccggagcgacaggtgatttggcttctgcacagttaggaggagcaccaaaccgatgggaggttttgtcagccacacctacaactataaaagatgaagctggtaatctagtccagattccaagtgctgctacttcaagtgggcagtatgttcttccccttcagaatttgcagaatcaacaaatattttccgttgcaccaggatcagattcatcaaatggtacagtgtccagtgttcaatatcaagtgataccacagatccagtcagcagatggtcagcaggttcaaattggtttcacaggctcttcagataatgggggtataaatcaagaaagcagtcaaattcagatcattcctggctctaatcaaaccttacttgcctctggaacaccttctgctaacatccagaatctcataccacagactggtcaagtccaggttcagggagttgcaattggtggttcatcttttcctggtcaaacccaagtagttgctaatgtgcctcttggtctgccaggaaatattacgtttgtaccaatcaatagtgtcgatctagattctttgggactctcgggcagttctcagacaatgactgcaggcattaatgccgacggacatttgataaacacaggacaagctatggatagttcagacaattcagaaaggactggtgagcgggtttctcctgatattaatgaaactaatactgatacagatttatttgtgccaacatcctcttcatcacagttgcctgttacgatagatagtacaggtatattacaacaaaacacaaatagcttgactacatctagtgggcaggttcattcttcagatcttcagggaaattatatccagtcgcctgtttctgaagagacacaggcacagaatattcaggtttctacagcacagcctgttgtacagcatctacaacttcaagagtctcagcagccaaccagtcaagcccaaattgtgcaaggtattacaccacagacaatccatggtgtgcaagccagtggtcaaaatatatcacaacaggctttgcaaaatcttcagttgcagctgaatcctggaacctttttaattcaggcacagacagtgaccccttctggacaggtaacttggcaaacgtttcaagtacaaggggtccagaacttgcagaatttgcaaatacagaatactgctgcccaacaaataactttgacgcctgttcaaaccctcacacttggtcaagttgcggcaggtggagccttcacttcaactccagttagtctaagcactggtcagttgccaaatctacaaacagttacagtgaactctatagattctgctggtatacagctacatccaggagagaatgctgacagtcctgcagatattaggatcaaggaagaagaacctgatcctgaagagtggcagctcagtggtgattctaccttgaataccaatgacctaacacacttaagagtacaggtggtagatgaagaaggggaccaacaacatcaagaaggaaaaagacttcggagggtagcttgcacctgtcccaactgtaaagaaggtggtggaagaggtaccaatcttgggaaaaagaagcaacacatttgtcatataccaggatgtggtaaagtctatgggaagacctcacatctgagagctcatctgcgttggcattctggagaacgcccttttgtttgtaactggatgtactgtggtaaaagatttactcgaagtgatgaattacagaggcacagaagaacacatacaggtgagaagaaatttgtttgtccagaatgttcaaaacgctttatgagaagtgaccaccttgccaaacatattaaaacacaccagaataaaaaaggtattcactctagcagtacagtgctggcatctgtggaagctgcgcgagatgatactttgattactgcaggaggaacaacgcttatccttgcaaatattcaacaaggttctgtttcagggataggaactgttaatacttccgccaccagcaatcaagatatccttaccaacactgaaatacctttacagcttgtcacagtttctggaaatgagacaatggagtaaatattacacaaatacttattcattgtggttatttttatacagtagtgagaagaatattgttcctaagttcttagatatctttttattgatgtgcaaaaatttttggattgacagtaacttggttatacatgacactgaaatgccttactttgtatgatattccatagtatattaaaaatggtaaaattgcatgggttttgtaggtacttttggaatctagaagaaatgaaattttaccaagttatataaagagaaaattgaatttaacaatgcgaatggtagtctaaccaaatgcatcaatcctgtgtggtttagtgtaaaaatgagaacatgttggtatttatctattgtaagataaaaaagctggtgggtgaaagaaatcatgttatgataaaaaattttgtaattttcttgatgactggaatttttattatgcataactgacaaatcaagtttccaagcaaatgttacatagtgtaggctttacttagcttatcaatttgtcattttgaagctaattattttaattaggttaactatgtacaatattttaagcattactcttgtaagattttgaaaactacattttaacatggaactctagggatagtcaccttttaaatcctgttgaaaagccatgtttaagatttaatttgccaaaataatgtcttgttaatattctttcaataacgaagttgggcaatataaccaatgtttaaaaaagtttaaaatgtataagttgaggcatttgggtggtaagagaatgttatagtgaattatcccttttcttgactattggaggaccaaaaaaataaggtgtattgcgtcttagcagtgatttttatccaatcttgtttccaaaaaccatgtctcccagggccttaaaagccatcatgtaaattaccagtaaagtgtaacatatgcaaacataacaaaatcacttccatagtgacgatactccaaccatatggatattagtcatagaagaactagaggttttatgatatttttttaagtcttttttttttttgtctaggtagtcagtctgcacttaaatatcaatcattttccttttttgcttcttcccttaaaatttatatgtatccagtacatttaattgagaagcgtatgttttttattatgctgtattttctttttattttttaattattgtttatattttcaattcaaaaatgtacaaaataaagttacattgctggtcttgtaagagctatacagttttcctaaatgtatacctgtaactgcagcagttcacctatttcaaaaatttggaattctgttcatttgttattcttaagaccacctcaaatttaaaggctaccttattgtacgtttaaagtgtattataacagtgtggtagttaataaaacactatttttttttcttttg

SEQ ID NO: 4 - protein, 496 aa, Homo sapiens
MTAGINADGHLINTGQAMDSSDNSERTGERVSPDINETNTDTDLFVPTSSSSQLPVTIDSTGILQQNTNSLTTSSGQVHSSDLQGNYIQSPVSEETQAQNIQVSTAQPVVQHLQLQESQQPTSQAQIVQGITPQTIHGVQASGQNISQQALQNLQLQLNPGTFLIQAQTVTPSGQVTWQTFQVQGVQNLQNLQIQNTAAQQITLTPVQTLTLGQVAAGGAFTSTPVSLSTGQLPNLQTVTVNSIDSAGIQLHPGENADSPADIRIKEEEPDPEEWQLSGDSTLNTNDLTHLRVQVVDEEGDQQHQEGKRLRRVACTCPNCKEGGGRGTNLGKKKQHICHIPGCGKVYGKTSHLRAHLRWHSGERPFVCNWMYCGKRFTRSDELQRHRRTHTGEKKFVCPECSKRFMRSDHLAKHIKTHQNKKGIHSSSTVLASVEAARDDTLITAGGTTLILANIQQGSVSGIGTVNTSATSNQDILTNTEIPLQLVTVSGNETME

SEQ ID NO: 5 - DNA, 25 bp, Artificial Sequence
  Description of Artificial Sequence Synthetic oligonucleotide
tgggtggttggtggtgggtggtggg

SEQ ID NO: 6 - DNA, 18 bp, Artificial Sequence
  Description of Artificial Sequence Synthetic oligonucleotide
taatacgactcactatag

SEQ ID NO: 7 - DNA, 20 bp, Artificial Sequence
  Description of Artificial Sequence Synthetic oligonucleotide
tggcaataatgggggcaatg

SEQ ID NO: 8 - DNA, 21 bp, Artificial Sequence
  Description of Artificial Sequence Synthetic oligonucleotide
tgaggtcaagctcacctgttc

SEQ ID NO: 9 - DNA, 18 bp, Artificial Sequence
  Description of Artificial Sequence Synthetic oligonucleotide
taatacgactcactatag

SEQ ID NO: 10 - DNA, 20 bp, Artificial Sequence
  Description of Artificial Sequence Synthetic oligonucleotide
atacttacctggcaggggag

SEQ ID NO: 11 - DNA, 21 bp, Artificial Sequence
  Description of Artificial Sequence Synthetic oligonucleotide
caggggaaagcgcgaacgcag

SEQ ID NO: 12 - DNA, 21 bp, Artificial Sequence
  Description of Artificial Sequence Synthetic oligonucleotide
attcgatggggcggggcgagc

SEQ ID NO: 13 - DNA, 21 bp, Artificial Sequence
  Description of Artificial Sequence Synthetic oligonucleotide
attcgatggttaagtgcgagc

SEQ ID NO: 14 - RNA, 22 nt, Artificial Sequence
  Description of Artificial Sequence Synthetic oligonucleotide
cugaugagcccgugaggacgaa

SEQ ID NO: 15 - DNA, 19 bp, Artificial Sequence
  Description of Artificial Sequence Synthetic oligonucleotide
taatggtggtggtgccttt

SEQ ID NO: 16 - DNA, 20 bp, Artificial Sequence
  Description of Artificial Sequence Synthetic oligonucleotide
gagatgatctgccagccatt

SEQ ID NO: 17 - DNA, 23 bp, Artificial Sequence
  Description of Artificial Sequence Synthetic oligonucleotide
aggccaaccgcgagaagatgacc

SEQ ID NO: 18 - DNA, 20 bp, Artificial Sequence
  Description of Artificial Sequence Synthetic oligonucleotide
gaagtccagggcgacgtagc

SEQ ID NO: 19 - RNA, 222 nt, Homo sapiens
auacuuacguggcaggggagauaccaugaucacgaaggugguuuucccagggcgaggcuuauccauuggacuccggaugugcugaccccugcgauuuccccaaaugugggaaacucgacugcagaauugagggaaaggggauggcuggcugaugaguccgugaggacgaaacuccugcccuccuccuccacuagugggggacugcguucgcgcuuuccccug

SEQ ID NO: 20 - RNA, 45 nt, Homo sapiens
uggaggaggagggcaggagucccagccagccauccccuuuggcuc

SEQ ID NO: 21 - RNA, 41 nt, Homo sapiens
ugaguugaacuccaguuaguguaagcacuggucaguugcca

SEQ ID NO: 22 - RNA, 223 nt, Homo sapiens
cauacuuaccuggcaggggagauaccaugaucacgaaggugguuuucccagggcgaggcuuauccauugcacuccggaugugcugaccccugcgauuuccccaaacgugggaaaucgacugcacaauuacccaaagggguggcuggcugaugagugcgagacgacgaaacuccugccccuccuccuccacccacuagugggggacugcguucgcgcuuucccu